SEQUENCE LISTING (288 sequences)

SEQ ID NO: 1 - RNA, 19 nt, Artificial Sequence
  Description of Artificial Sequence Synthetic oligonucleotide
cccucagcaaggacagcag

SEQ ID NO: 2 - RNA, 19 nt, Artificial Sequence
  Description of Artificial Sequence Synthetic oligonucleotide
gaggaccagcuaagaggga

SEQ ID NO: 3 - RNA, 19 nt, Artificial Sequence
  Description of Artificial Sequence Synthetic oligonucleotide
agagaagcaacuacagacc

SEQ ID NO: 4 - RNA, 19 nt, Artificial Sequence
  Description of Artificial Sequence Synthetic oligonucleotide
ccccccugaaaacaacccu

SEQ ID NO: 5 - RNA, 19 nt, Artificial Sequence
  Description of Artificial Sequence Synthetic oligonucleotide
ucagacgccacauccccug

SEQ ID NO: 6 - RNA, 19 nt, Artificial Sequence
  Description of Artificial Sequence Synthetic oligonucleotide
gacaagcugccaggcaggu

SEQ ID NO: 7 - RNA, 19 nt, Artificial Sequence
  Description of Artificial Sequence Synthetic oligonucleotide
uucucuuccucucacauac

SEQ ID NO: 8 - RNA, 19 nt, Artificial Sequence
  Description of Artificial Sequence Synthetic oligonucleotide
cugacccacggcuccaccc

SEQ ID NO: 9 - RNA, 19 nt, Artificial Sequence
  Description of Artificial Sequence Synthetic oligonucleotide
cucucuccccuggaaagga

SEQ ID NO: 10 - RNA, 19 nt, Artificial Sequence
  Description of Artificial Sequence Synthetic oligonucleotide
acaccaugagcacugaaag

SEQ ID NO: 11 - RNA, 19 nt, Artificial Sequence
  Description of Artificial Sequence Synthetic oligonucleotide
gcaugauccgggacgugga

SEQ ID NO: 12 - RNA, 19 nt, Artificial Sequence
  Description of Artificial Sequence Synthetic oligonucleotide
agcuggccgaggaggcgcu

SEQ ID NO: 13 - RNA, 19 nt, Artificial Sequence
  Description of Artificial Sequence Synthetic oligonucleotide
uccccaagaagacaggggg

SEQ ID NO: 14 - RNA, 19 nt, Artificial Sequence
  Description of Artificial Sequence Synthetic oligonucleotide
ggccccagggcuccaggcg

SEQ ID NO: 15 - RNA, 19 nt, Artificial Sequence
  Description of Artificial Sequence Synthetic oligonucleotide
ggugcuuguuccucagccu

SEQ ID NO: 16 - RNA, 19 nt, Artificial Sequence
  Description of Artificial Sequence Synthetic oligonucleotide
ucuucuccuuccugaucgu

SEQ ID NO: 17 - RNA, 19 nt, Artificial Sequence
  Description of Artificial Sequence Synthetic oligonucleotide
uggcaggcgccaccacgcu

SEQ ID NO: 18 - RNA, 19 nt, Artificial Sequence
  Description of Artificial Sequence Synthetic oligonucleotide
ucuucugccugcugcacuu

SEQ ID NO: 19 - RNA, 19 nt, Artificial Sequence
  Description of Artificial Sequence Synthetic oligonucleotide
uuggagugaucggccccca

SEQ ID NO: 20 - RNA, 19 nt, Artificial Sequence
  Description of Artificial Sequence Synthetic oligonucleotide
agagggaagaguuccccag

SEQ ID NO: 21 - RNA, 19 nt, Artificial Sequence
  Description of Artificial Sequence Synthetic oligonucleotide
gggaccucucucuaaucag

SEQ ID NO: 22 - RNA, 19 nt, Artificial Sequence
  Description of Artificial Sequence Synthetic oligonucleotide
gcccucuggcccaggcagu

SEQ ID NO: 23 - RNA, 19 nt, Artificial Sequence
  Description of Artificial Sequence Synthetic oligonucleotide
ucagaucaucuucucgaac

SEQ ID NO: 24 - RNA, 19 nt, Artificial Sequence
  Description of Artificial Sequence Synthetic oligonucleotide
ccccgagugacaagccugu

SEQ ID NO: 25 - RNA, 19 nt, Artificial Sequence
  Description of Artificial Sequence Synthetic oligonucleotide
uagcccauguuguagcaaa

SEQ ID NO: 26 - RNA, 19 nt, Artificial Sequence
  Description of Artificial Sequence Synthetic oligonucleotide
acccucaagcugaggggca

SEQ ID NO: 27 - RNA, 19 nt, Artificial Sequence
  Description of Artificial Sequence Synthetic oligonucleotide
agcuccaguggcugaaccg

SEQ ID NO: 28 - RNA, 19 nt, Artificial Sequence
  Description of Artificial Sequence Synthetic oligonucleotide
gccgggccaaugcccuccu

SEQ ID NO: 29 - RNA, 19 nt, Artificial Sequence
  Description of Artificial Sequence Synthetic oligonucleotide
uggccaauggcguggagcu

SEQ ID NO: 30 - RNA, 19 nt, Artificial Sequence
  Description of Artificial Sequence Synthetic oligonucleotide
ugagagauaaccagcuggu

SEQ ID NO: 31 - RNA, 19 nt, Artificial Sequence
  Description of Artificial Sequence Synthetic oligonucleotide
uggugccaucagagggccu

SEQ ID NO: 32 - RNA, 19 nt, Artificial Sequence
  Description of Artificial Sequence Synthetic oligonucleotide
uguaccucaucuacuccca

SEQ ID NO: 33 - RNA, 19 nt, Artificial Sequence
  Description of Artificial Sequence Synthetic oligonucleotide
agguccucuucaagggcca

SEQ ID NO: 34 - RNA, 19 nt, Artificial Sequence
  Description of Artificial Sequence Synthetic oligonucleotide
aaggcugccccuccaccca

SEQ ID NO: 35 - RNA, 19 nt, Artificial Sequence
  Description of Artificial Sequence Synthetic oligonucleotide
augugcuccucacccacac

SEQ ID NO: 36 - RNA, 19 nt, Artificial Sequence
  Description of Artificial Sequence Synthetic oligonucleotide
ccaucagccgcaucgccgu

SEQ ID NO: 37 - RNA, 19 nt, Artificial Sequence
  Description of Artificial Sequence Synthetic oligonucleotide
ucuccuaccagaccaaggu

SEQ ID NO: 38 - RNA, 19 nt, Artificial Sequence
  Description of Artificial Sequence Synthetic oligonucleotide
ucaaccuccucucugccau

SEQ ID NO: 39 - RNA, 19 nt, Artificial Sequence
  Description of Artificial Sequence Synthetic oligonucleotide
ucaagagccccugccagag

SEQ ID NO: 40 - RNA, 19 nt, Artificial Sequence
  Description of Artificial Sequence Synthetic oligonucleotide
gggagaccccagagggggc

SEQ ID NO: 41 - RNA, 19 nt, Artificial Sequence
  Description of Artificial Sequence Synthetic oligonucleotide
cugaggccaagcccuggua

SEQ ID NO: 42 - RNA, 19 nt, Artificial Sequence
  Description of Artificial Sequence Synthetic oligonucleotide
augagcccaucuaucuggg

SEQ ID NO: 43 - RNA, 19 nt, Artificial Sequence
  Description of Artificial Sequence Synthetic oligonucleotide
gaggggucuuccagcugga

SEQ ID NO: 44 - RNA, 19 nt, Artificial Sequence
  Description of Artificial Sequence Synthetic oligonucleotide
agaagggugaccgacucag

SEQ ID NO: 45 - RNA, 19 nt, Artificial Sequence
  Description of Artificial Sequence Synthetic oligonucleotide
gcgcugagaucaaucggcc

SEQ ID NO: 46 - RNA, 19 nt, Artificial Sequence
  Description of Artificial Sequence Synthetic oligonucleotide
ccgacuaucucgacuuugc

SEQ ID NO: 47 - RNA, 19 nt, Artificial Sequence
  Description of Artificial Sequence Synthetic oligonucleotide
ccgagucugggcaggucua

SEQ ID NO: 48 - RNA, 19 nt, Artificial Sequence
  Description of Artificial Sequence Synthetic oligonucleotide
acuuugggaucauugcccu

SEQ ID NO: 49 - RNA, 19 nt, Artificial Sequence
  Description of Artificial Sequence Synthetic oligonucleotide
ugugaggaggacgaacauc

SEQ ID NO: 50 - RNA, 19 nt, Artificial Sequence
  Description of Artificial Sequence Synthetic oligonucleotide
ccaaccuucccaaacgccu

SEQ ID NO: 51 - RNA, 19 nt, Artificial Sequence
  Description of Artificial Sequence Synthetic oligonucleotide
uccccugccccaaucccuu

SEQ ID NO: 52 - RNA, 19 nt, Artificial Sequence
  Description of Artificial Sequence Synthetic oligonucleotide
uuauuacccccuccuucag

SEQ ID NO: 53 - RNA, 19 nt, Artificial Sequence
  Description of Artificial Sequence Synthetic oligonucleotide
gacacccucaaccucuucu

SEQ ID NO: 54 - RNA, 19 nt, Artificial Sequence
  Description of Artificial Sequence Synthetic oligonucleotide
uggcucaaaaagagaauug

SEQ ID NO: 55 - RNA, 19 nt, Artificial Sequence
  Description of Artificial Sequence Synthetic oligonucleotide
gggggcuuagggucggaac

SEQ ID NO: 56 - RNA, 19 nt, Artificial Sequence
  Description of Artificial Sequence Synthetic oligonucleotide
cccaagcuuagaacuuuaa

SEQ ID NO: 57 - RNA, 19 nt, Artificial Sequence
  Description of Artificial Sequence Synthetic oligonucleotide
agcaacaagaccaccacuu

SEQ ID NO: 58 - RNA, 19 nt, Artificial Sequence
  Description of Artificial Sequence Synthetic oligonucleotide
ucgaaaccugggauucagg

SEQ ID NO: 59 - RNA, 19 nt, Artificial Sequence
  Description of Artificial Sequence Synthetic oligonucleotide
gaauguguggccugcacag

SEQ ID NO: 60 - RNA, 19 nt, Artificial Sequence
  Description of Artificial Sequence Synthetic oligonucleotide
gugaagugcuggcaaccac

SEQ ID NO: 61 - RNA, 19 nt, Artificial Sequence
  Description of Artificial Sequence Synthetic oligonucleotide
cuaagaauucaaacugggg

SEQ ID NO: 62 - RNA, 19 nt, Artificial Sequence
  Description of Artificial Sequence Synthetic oligonucleotide
gccuccagaacucacuggg

SEQ ID NO: 63 - RNA, 19 nt, Artificial Sequence
  Description of Artificial Sequence Synthetic oligonucleotide
ggccuacagcuuugauccc

SEQ ID NO: 64 - RNA, 19 nt, Artificial Sequence
  Description of Artificial Sequence Synthetic oligonucleotide
cugacaucuggaaucugga

SEQ ID NO: 65 - RNA, 19 nt, Artificial Sequence
  Description of Artificial Sequence Synthetic oligonucleotide
agaccagggagccuuuggu

SEQ ID NO: 66 - RNA, 19 nt, Artificial Sequence
  Description of Artificial Sequence Synthetic oligonucleotide
uucuggccagaaugcugca

SEQ ID NO: 67 - RNA, 19 nt, Artificial Sequence
  Description of Artificial Sequence Synthetic oligonucleotide
aggacuugagaagaccuca

SEQ ID NO: 68 - RNA, 19 nt, Artificial Sequence
  Description of Artificial Sequence Synthetic oligonucleotide
accuagaaauugacacaag

SEQ ID NO: 69 - RNA, 19 nt, Artificial Sequence
  Description of Artificial Sequence Synthetic oligonucleotide
guggaccuuaggccuuccu

SEQ ID NO: 70 - RNA, 19 nt, Artificial Sequence
  Description of Artificial Sequence Synthetic oligonucleotide
ucucuccagauguuuccag

SEQ ID NO: 71 - RNA, 19 nt, Artificial Sequence
  Description of Artificial Sequence Synthetic oligonucleotide
gacuuccuugagacacgga

SEQ ID NO: 72 - RNA, 19 nt, Artificial Sequence
  Description of Artificial Sequence Synthetic oligonucleotide
agcccagcccuccccaugg

SEQ ID NO: 73 - RNA, 19 nt, Artificial Sequence
  Description of Artificial Sequence Synthetic oligonucleotide
gagccagcucccucuauuu

SEQ ID NO: 74 - RNA, 19 nt, Artificial Sequence
  Description of Artificial Sequence Synthetic oligonucleotide
uauguuugcacuugugauu

SEQ ID NO: 75 - RNA, 19 nt, Artificial Sequence
  Description of Artificial Sequence Synthetic oligonucleotide
uauuuauuauuuauuuauu

SEQ ID NO: 76 - RNA, 19 nt, Artificial Sequence
  Description of Artificial Sequence Synthetic oligonucleotide
uauuuauuuauuuacagau

SEQ ID NO: 77 - RNA, 19 nt, Artificial Sequence
  Description of Artificial Sequence Synthetic oligonucleotide
ugaauguauuuauuuggga

SEQ ID NO: 78 - RNA, 19 nt, Artificial Sequence
  Description of Artificial Sequence Synthetic oligonucleotide
agaccgggguauccugggg

SEQ ID NO: 79 - RNA, 19 nt, Artificial Sequence
  Description of Artificial Sequence Synthetic oligonucleotide
ggacccaauguaggagcug

SEQ ID NO: 80 - RNA, 19 nt, Artificial Sequence
  Description of Artificial Sequence Synthetic oligonucleotide
gccuuggcucagacauguu

SEQ ID NO: 81 - RNA, 19 nt, Artificial Sequence
  Description of Artificial Sequence Synthetic oligonucleotide
uuuccgugaaaacggagcu

SEQ ID NO: 82 - RNA, 19 nt, Artificial Sequence
  Description of Artificial Sequence Synthetic oligonucleotide
ugaacaauaggcuguuccc

SEQ ID NO: 83 - RNA, 19 nt, Artificial Sequence
  Description of Artificial Sequence Synthetic oligonucleotide
cauguagcccccuggccuc

SEQ ID NO: 84 - RNA, 19 nt, Artificial Sequence
  Description of Artificial Sequence Synthetic oligonucleotide
cugugccuucuuuugauua

SEQ ID NO: 85 - RNA, 19 nt, Artificial Sequence
  Description of Artificial Sequence Synthetic oligonucleotide
auguuuuuuaaaauauuua

SEQ ID NO: 86 - RNA, 19 nt, Artificial Sequence
  Description of Artificial Sequence Synthetic oligonucleotide
aucugauuaaguugucuaa

SEQ ID NO: 87 - RNA, 19 nt, Artificial Sequence
  Description of Artificial Sequence Synthetic oligonucleotide
aacaaugcugauuugguga

SEQ ID NO: 88 - RNA, 19 nt, Artificial Sequence
  Description of Artificial Sequence Synthetic oligonucleotide
accaacugucacucauugc

SEQ ID NO: 89 - RNA, 19 nt, Artificial Sequence
  Description of Artificial Sequence Synthetic oligonucleotide
cugagccucugcuccccag

SEQ ID NO: 90 - RNA, 19 nt, Artificial Sequence
  Description of Artificial Sequence Synthetic oligonucleotide
ggggaguugugucuguaau

SEQ ID NO: 91 - RNA, 19 nt, Artificial Sequence
  Description of Artificial Sequence Synthetic oligonucleotide
ucgcccuacuauucagugg

SEQ ID NO: 92 - RNA, 19 nt, Artificial Sequence
  Description of Artificial Sequence Synthetic oligonucleotide
gcgagaaauaaaguuugcu

SEQ ID NO: 93 - RNA, 19 nt, Artificial Sequence
  Description of Artificial Sequence Synthetic oligonucleotide
uaaaguuugcuuagaaaag

SEQ ID NO: 94 - RNA, 19 nt, Artificial Sequence
  Description of Artificial Sequence Synthetic oligonucleotide
cacccugacaagcugccag

SEQ ID NO: 95 - RNA, 19 nt, Artificial Sequence
  Description of Artificial Sequence Synthetic oligonucleotide
ggcagguucucuuccucuc

SEQ ID NO: 96 - RNA, 19 nt, Artificial Sequence
  Description of Artificial Sequence Synthetic oligonucleotide
cacauacugacccacggcu

SEQ ID NO: 97 - RNA, 19 nt, Artificial Sequence
  Description of Artificial Sequence Synthetic oligonucleotide
uccacccucucuccccugg

SEQ ID NO: 98 - RNA, 19 nt, Artificial Sequence
  Description of Artificial Sequence Synthetic oligonucleotide
gaaaggacaccaugagcac

SEQ ID NO: 99 - RNA, 19 nt, Artificial Sequence
  Description of Artificial Sequence Synthetic oligonucleotide
cugaaagcaugauccggga

SEQ ID NO: 100 - RNA, 19 nt, Artificial Sequence
  Description of Artificial Sequence Synthetic oligonucleotide
acguggagcuggccgagga

SEQ ID NO: 101 - RNA, 19 nt, Artificial Sequence
  Description of Artificial Sequence Synthetic oligonucleotide
aggcgcuccccaagaagac

SEQ ID NO: 102 - RNA, 19 nt, Artificial Sequence
  Description of Artificial Sequence Synthetic oligonucleotide
caggggggccccagggcuc

SEQ ID NO: 103 - RNA, 19 nt, Artificial Sequence
  Description of Artificial Sequence Synthetic oligonucleotide
ccaggcggugcuuguuccu

SEQ ID NO: 104 - RNA, 19 nt, Artificial Sequence
  Description of Artificial Sequence Synthetic oligonucleotide
ucagccucuucuccuuccu

SEQ ID NO: 105 - RNA, 19 nt, Artificial Sequence
  Description of Artificial Sequence Synthetic oligonucleotide
ugaucguggcaggcgccac

SEQ ID NO: 106 - RNA, 19 nt, Artificial Sequence
  Description of Artificial Sequence Synthetic oligonucleotide
ccacgcucuucugccugcu

SEQ ID NO: 107 - RNA, 19 nt, Artificial Sequence
  Description of Artificial Sequence Synthetic oligonucleotide
ugcacuuuggagugaucgg

SEQ ID NO: 108 - RNA, 19 nt, Artificial Sequence
  Description of Artificial Sequence Synthetic oligonucleotide
gcccccagagggaagaguc

SEQ ID NO: 109 - RNA, 19 nt, Artificial Sequence
  Description of Artificial Sequence Synthetic oligonucleotide
cccccagggaccucucucu

SEQ ID NO: 110 - RNA, 19 nt, Artificial Sequence
  Description of Artificial Sequence Synthetic oligonucleotide
uaaucagcccucuggccca

SEQ ID NO: 111 - RNA, 19 nt, Artificial Sequence
  Description of Artificial Sequence Synthetic oligonucleotide
aggcagucagaucaucuuc

SEQ ID NO: 112 - RNA, 19 nt, Artificial Sequence
  Description of Artificial Sequence Synthetic oligonucleotide
cucgaaccccgagugacaa

SEQ ID NO: 113 - RNA, 19 nt, Artificial Sequence
  Description of Artificial Sequence Synthetic oligonucleotide
agccuguagcccauguugu

SEQ ID NO: 114 - RNA, 19 nt, Artificial Sequence
  Description of Artificial Sequence Synthetic oligonucleotide
uagcaaacccucaagcuga

SEQ ID NO: 115 - RNA, 19 nt, Artificial Sequence
  Description of Artificial Sequence Synthetic oligonucleotide
aggggcagcuccaguggcu

SEQ ID NO: 116 - RNA, 19 nt, Artificial Sequence
  Description of Artificial Sequence Synthetic oligonucleotide
ugaaccgccgggccaaugc

SEQ ID NO: 117 - RNA, 19 nt, Artificial Sequence
  Description of Artificial Sequence Synthetic oligonucleotide
cccuccuggccaauggcgu

SEQ ID NO: 118 - RNA, 19 nt, Artificial Sequence
  Description of Artificial Sequence Synthetic oligonucleotide
uggagcugagagauaacca

SEQ ID NO: 119 - RNA, 19 nt, Artificial Sequence
  Description of Artificial Sequence Synthetic oligonucleotide
agcugguggugccaucaga

SEQ ID NO: 120 - RNA, 19 nt, Artificial Sequence
  Description of Artificial Sequence Synthetic oligonucleotide
agggccuguaccucaucua

SEQ ID NO: 121 - RNA, 19 nt, Artificial Sequence
  Description of Artificial Sequence Synthetic oligonucleotide
acucccagguccucuucaa

SEQ ID NO: 122 - RNA, 19 nt, Artificial Sequence
  Description of Artificial Sequence Synthetic oligonucleotide
agggccaaggcugccccuc

SEQ ID NO: 123 - RNA, 19 nt, Artificial Sequence
  Description of Artificial Sequence Synthetic oligonucleotide
ccacccaugugcuccucac

SEQ ID NO: 124 - RNA, 19 nt, Artificial Sequence
  Description of Artificial Sequence Synthetic oligonucleotide
cccacaccaucagccgcau

SEQ ID NO: 125 - RNA, 19 nt, Artificial Sequence
  Description of Artificial Sequence Synthetic oligonucleotide
ucgccgucuccuaccagac

SEQ ID NO: 126 - RNA, 19 nt, Artificial Sequence
  Description of Artificial Sequence Synthetic oligonucleotide
ccaaggucaaccuccucuc

SEQ ID NO: 127 - RNA, 19 nt, Artificial Sequence
  Description of Artificial Sequence Synthetic oligonucleotide
cugccaucaagagccccug

SEQ ID NO: 128 - RNA, 19 nt, Artificial Sequence
  Description of Artificial Sequence Synthetic oligonucleotide
gccagagggagaccccaga

SEQ ID NO: 129 - RNA, 19 nt, Artificial Sequence
  Description of Artificial Sequence Synthetic oligonucleotide
agggggcugaggccaagcc

SEQ ID NO: 130 - RNA, 19 nt, Artificial Sequence
  Description of Artificial Sequence Synthetic oligonucleotide
ccugguaugagcccaucua

SEQ ID NO: 131 - RNA, 19 nt, Artificial Sequence
  Description of Artificial Sequence Synthetic oligonucleotide
aucugggaggggucuucca

SEQ ID NO: 132 - RNA, 19 nt, Artificial Sequence
  Description of Artificial Sequence Synthetic oligonucleotide
agcuggagaagggugaccg

SEQ ID NO: 133 - RNA, 19 nt, Artificial Sequence
  Description of Artificial Sequence Synthetic oligonucleotide
gacucagcgcugagaucaa

SEQ ID NO: 134 - RNA, 19 nt, Artificial Sequence
  Description of Artificial Sequence Synthetic oligonucleotide
aucggcccgacuaucucga

SEQ ID NO: 135 - RNA, 19 nt, Artificial Sequence
  Description of Artificial Sequence Synthetic oligonucleotide
acuuugccgagucugggca

SEQ ID NO: 136 - RNA, 19 nt, Artificial Sequence
  Description of Artificial Sequence Synthetic oligonucleotide
aggucuacuuugggaucau

SEQ ID NO: 137 - RNA, 19 nt, Artificial Sequence
  Description of Artificial Sequence Synthetic oligonucleotide
uugcccugugaggaggacg

SEQ ID NO: 138 - RNA, 19 nt, Artificial Sequence
  Description of Artificial Sequence Synthetic oligonucleotide
gaacauccaaccuucccaa

SEQ ID NO: 139 - RNA, 19 nt, Artificial Sequence
  Description of Artificial Sequence Synthetic oligonucleotide
aacgccuccccugccccaa

SEQ ID NO: 140 - RNA, 19 nt, Artificial Sequence
  Description of Artificial Sequence Synthetic oligonucleotide
aucccuuuauuacccccuc

SEQ ID NO: 141 - RNA, 19 nt, Artificial Sequence
  Description of Artificial Sequence Synthetic oligonucleotide
ccuucagacacccucaacc

SEQ ID NO: 142 - RNA, 19 nt, Artificial Sequence
  Description of Artificial Sequence Synthetic oligonucleotide
cucuucuggcucaaaaaga

SEQ ID NO: 143 - RNA, 19 nt, Artificial Sequence
  Description of Artificial Sequence Synthetic oligonucleotide
agaauugggggcuuagggu

SEQ ID NO: 144 - RNA, 19 nt, Artificial Sequence
  Description of Artificial Sequence Synthetic oligonucleotide
ucggaacccaagcuuagaa

SEQ ID NO: 145 - RNA, 19 nt, Artificial Sequence
  Description of Artificial Sequence Synthetic oligonucleotide
acuuuaagcaacaagacca

SEQ ID NO: 146 - RNA, 19 nt, Artificial Sequence
  Description of Artificial Sequence Synthetic oligonucleotide
accacuucgaaaccuggga

SEQ ID NO: 147 - RNA, 19 nt, Artificial Sequence
  Description of Artificial Sequence Synthetic oligonucleotide
auucaggaauguguggccu

SEQ ID NO: 148 - RNA, 19 nt, Artificial Sequence
  Description of Artificial Sequence Synthetic oligonucleotide
ugcacagugaagugcuggc

SEQ ID NO: 149 - RNA, 19 nt, Artificial Sequence
  Description of Artificial Sequence Synthetic oligonucleotide
caaccacuaagaauucaaa

SEQ ID NO: 150 - RNA, 19 nt, Artificial Sequence
  Description of Artificial Sequence Synthetic oligonucleotide
acuggggccuccagaacuc

SEQ ID NO: 151 - RNA, 19 nt, Artificial Sequence
  Description of Artificial Sequence Synthetic oligonucleotide
cacuggggccuacagcuuu

SEQ ID NO: 152 - RNA, 19 nt, Artificial Sequence
  Description of Artificial Sequence Synthetic oligonucleotide
ugaucccugacaucuggaa

SEQ ID NO: 153 - RNA, 19 nt, Artificial Sequence
  Description of Artificial Sequence Synthetic oligonucleotide
aucuggagaccagggagcc

SEQ ID NO: 154 - RNA, 19 nt, Artificial Sequence
  Description of Artificial Sequence Synthetic oligonucleotide
cuuugguucuggccagaau

SEQ ID NO: 155 - RNA, 19 nt, Artificial Sequence
  Description of Artificial Sequence Synthetic oligonucleotide
ugcugcaggacuugagaag

SEQ ID NO: 156 - RNA, 19 nt, Artificial Sequence
  Description of Artificial Sequence Synthetic oligonucleotide
gaccucaccuagaaauuga

SEQ ID NO: 157 - RNA, 19 nt, Artificial Sequence
  Description of Artificial Sequence Synthetic oligonucleotide
acacaaguggaccuuaggc

SEQ ID NO: 158 - RNA, 19 nt, Artificial Sequence
  Description of Artificial Sequence Synthetic oligonucleotide
ccuuccucucuccagaugu

SEQ ID NO: 159 - RNA, 19 nt, Artificial Sequence
  Description of Artificial Sequence Synthetic oligonucleotide
uuuccagacuuccuugaga

SEQ ID NO: 160 - RNA, 19 nt, Artificial Sequence
  Description of Artificial Sequence Synthetic oligonucleotide
acacggagcccagcccucc

SEQ ID NO: 161 - RNA, 19 nt, Artificial Sequence
  Description of Artificial Sequence Synthetic oligonucleotide
cccauggagccagcucccu

SEQ ID NO: 162 - RNA, 19 nt, Artificial Sequence
  Description of Artificial Sequence Synthetic oligonucleotide
ucuauuuauguuugcacuu

SEQ ID NO: 163 - RNA, 19 nt, Artificial Sequence
  Description of Artificial Sequence Synthetic oligonucleotide
ugugauuauuuauuauuua

SEQ ID NO: 164 - RNA, 19 nt, Artificial Sequence
  Description of Artificial Sequence Synthetic oligonucleotide
auuuauuauuuauuuauuu

SEQ ID NO: 165 - RNA, 19 nt, Artificial Sequence
  Description of Artificial Sequence Synthetic oligonucleotide
uacagaugaauguauuuau

SEQ ID NO: 166 - RNA, 19 nt, Artificial Sequence
  Description of Artificial Sequence Synthetic oligonucleotide
uuugggagaccgggguauc

SEQ ID NO: 167 - RNA, 19 nt, Artificial Sequence
  Description of Artificial Sequence Synthetic oligonucleotide
ccugggggacccaauguag

SEQ ID NO: 168 - RNA, 19 nt, Artificial Sequence
  Description of Artificial Sequence Synthetic oligonucleotide
ggagcugccuuggcucaga

SEQ ID NO: 169 - RNA, 19 nt, Artificial Sequence
  Description of Artificial Sequence Synthetic oligonucleotide
acauguuuuccgugaaaac

SEQ ID NO: 170 - RNA, 19 nt, Artificial Sequence
  Description of Artificial Sequence Synthetic oligonucleotide
cggaggcugaacaauaggc

SEQ ID NO: 171 - RNA, 19 nt, Artificial Sequence
  Description of Artificial Sequence Synthetic oligonucleotide
cuguucccauguagccccc

SEQ ID NO: 172 - RNA, 19 nt, Artificial Sequence
  Description of Artificial Sequence Synthetic oligonucleotide
cuggccucugugccuucuu

SEQ ID NO: 173 - RNA, 19 nt, Artificial Sequence
  Description of Artificial Sequence Synthetic oligonucleotide
uuugauuauguuuuuuaaa

SEQ ID NO: 174 - RNA, 19 nt, Artificial Sequence
  Description of Artificial Sequence Synthetic oligonucleotide
aauauuaucugauuaaguu

SEQ ID NO: 175 - RNA, 19 nt, Artificial Sequence
  Description of Artificial Sequence Synthetic oligonucleotide
ugucuaaacaaugcugauu

SEQ ID NO: 176 - RNA, 19 nt, Artificial Sequence
  Description of Artificial Sequence Synthetic oligonucleotide
uuggugaccaacugucacu

SEQ ID NO: 177 - RNA, 19 nt, Artificial Sequence
  Description of Artificial Sequence Synthetic oligonucleotide
ucauugcugaggccucugc

SEQ ID NO: 178 - RNA, 19 nt, Artificial Sequence
  Description of Artificial Sequence Synthetic oligonucleotide
cuccccagggaguuguguc

SEQ ID NO: 179 - RNA, 19 nt, Artificial Sequence
  Description of Artificial Sequence Synthetic oligonucleotide
cuguaaucggccuacuauu

SEQ ID NO: 180 - RNA, 19 nt, Artificial Sequence
  Description of Artificial Sequence Synthetic oligonucleotide
ucaguggcgagaaauaaag

SEQ ID NO: 181 - RNA, 19 nt, Artificial Sequence
  Description of Artificial Sequence Synthetic oligonucleotide
uaaagguugcuuaggaaag

SEQ ID NO: 182 - RNA, 1669 nt, Homo sapiens
cucccucagcaaggacagcagaggaccagcuaagagggagagaagcaacuacagacccccccugaaaacaacccucagacgccacauccccugacaagcugccaggcagguucucuuccucucacauacugacccacggcuccacccucucuccccuggaaaggacaccaugagcacugaaagcaugauccgggacguggagcuggccgaggaggcgcuccccaagaagacaggggggccccagggcuccaggcggugcuuguuccucagccucuucuccuuccugaucguggcaggcgccaccacgcucuucugccugcugcacuuuggagugaucggcccccagagggaagaguuccccagggaccucucucuaaucagcccucuggcccaggcagucagaucaucuucucgaaccccgagugacaagccuguagcccauguuguagcaaacccucaagcugaggggcagcuccaguggcugaaccgccgggccaaugcccuccuggccaauggcguggagcugagagauaaccagcugguggugccaucagagggccuguaccucaucuacucccagguccucuucaagggccaaggcugccccuccacccaugugcuccucacccacaccaucagccgcaucgccgucuccuaccagaccaaggucaaccuccucucugccaucaagagccccugccagagggagaccccagagggggcugaggccaagcccugguaugagcccaucuaucugggaggggucuuccagcuggagaagggugaccgacucagcgcugagaucaaucggcccgacuaucucgacuuugccgagucugggcaggucuacuuugggaucauugcccugugaggaggacgaacauccaaccuucccaaacgccuccccugccccaaucccuuuauuacccccuccuucagacacccucaaccucuucuggcucaaaaagagaauugggggcuuagggucggaacccaagcuuagaacuuuaagcaacaagaccaccacuucgaaaccugggauucaggaauguguggccugcacagugaagugcuggcaaccacuaagaauucaaacuggggccuccagaacucacuggggccuacagcuuugaucccugacaucuggaaucuggagaccagggagccuuugguucuggccagaaugcugcaggacuugagaagaccucaccuagaaauugacacaaguggaccuuaggccuuccucucuccagauguuuccagacuuccuugagacacggagcccagcccuccccauggagccagcucccucuauuuauguuugcacuugugauuauuuauuauuuauuuauuauuuauuuauuuacagaugaauguauuuauuugggagaccgggguauccugggggacccaauguaggagcugccuuggcucagacauguuuuccgugaaaacggagcugaacaauaggcuguucccauguagcccccuggccucugugccuucuuuugauuauguuuuuuaaaauauuuaucugauuaaguugucuaaacaaugcugauuuggugaccaacugucacucauugcugagccucugcuccccaggggaguugugucuguaaucgcccuacuauucaguggcgagaaauaaaguuugcuuagaaaagaa

SEQ ID NO: 183 - RNA, 19 nt, Artificial Sequence
  Description of Artificial Sequence Synthetic oligonucleotide
uccucagccucuucuccuu

SEQ ID NO: 184 - RNA, 19 nt, Artificial Sequence
  Description of Artificial Sequence Synthetic oligonucleotide
gggucggaacccaagcuua

SEQ ID NO: 185 - RNA, 19 nt, Artificial Sequence
  Description of Artificial Sequence Synthetic oligonucleotide
gccuguagcccauguugua

SEQ ID NO: 186 - RNA, 21 nt, Artificial Sequence
  Description of Artificial Sequence Synthetic oligonucleotide
gccuguaccucaucuacucuu

SEQ ID NO: 187 - RNA, 25 nt, Artificial Sequence
  Description of Artificial Sequence Synthetic oligonucleotide
gccucuucuccuuccugaucguggc

SEQ ID NO: 188 - RNA, 25 nt, Artificial Sequence
  Description of Artificial Sequence Synthetic oligonucleotide
gccugcugcacuuuggagugaucgg

SEQ ID NO: 189 - DNA, 25 bp, Artificial Sequence
  Description of Combined DNA/RNA Molecule Synthetic oligonucleotide
cccaugugcuccucacccacaccat

SEQ ID NO: 190 - DNA, 25 bp, Artificial Sequence
  Description of Combined DNA/RNA Molecule Synthetic oligonucleotide
accucaucuacucccagguccuctt

SEQ ID NO: 191 - RNA, 21 nt, Artificial Sequence
  Description of Artificial Sequence Synthetic oligonucleotide
ccgacucagcgcugagaucaa

SEQ ID NO: 192 - DNA, 21 bp, Artificial Sequence
  Description of Combined DNA/RNA Molecule Synthetic oligonucleotide
gaugaggcauagcagcaggtt

SEQ ID NO: 193 - DNA, 21 bp, Artificial Sequence
  Description of Combined DNA/RNA Molecule Synthetic oligonucleotide
ccugcugcuaugccucauctt

SEQ ID NO: 194 - DNA, 21 bp, Artificial Sequence
  Description of Combined DNA/RNA Molecule Synthetic oligonucleotide
gaugaggcauagcagcaggtt

SEQ ID NO: 195 - DNA, 21 bp, Artificial Sequence
  Description of Combined DNA/RNA Molecule Synthetic oligonucleotide
ccugcugcuaugccucauctt

SEQ ID NO: 196 - DNA, 21 bp, Artificial Sequence
  Description of Combined DNA/RNA Molecule Synthetic oligonucleotide
gaugaggcauagcagcaggtt

SEQ ID NO: 197 - DNA, 21 bp, Artificial Sequence
  Description of Combined DNA/RNA Molecule Synthetic oligonucleotide
ccugcugcuaugccucauctt

SEQ ID NO: 198 - DNA, 21 bp, Artificial Sequence
  Description of Combined DNA/RNA Molecule Synthetic oligonucleotide
gaugaggcauagcagcaggtt

SEQ ID NO: 199 - DNA, 21 bp, Artificial Sequence
  Description of Combined DNA/RNA Molecule Synthetic oligonucleotide
ccugcugcuaugccucauctt

SEQ ID NO: 200 - DNA, 21 bp, Artificial Sequence
  Description of Combined DNA/RNA Molecule Synthetic oligonucleotide
gaugaggcauagcagcaggtt

SEQ ID NO: 201 - DNA, 21 bp, Artificial Sequence
  Description of Combined DNA/RNA Molecule Synthetic oligonucleotide
ccugcugcuaugccucauctt

SEQ ID NO: 202 - DNA, 21 bp, Artificial Sequence
  Description of Combined DNA/RNA Molecule Synthetic oligonucleotide
gaugaggcauagcagcaggtt

SEQ ID NO: 203 - DNA, 21 bp, Artificial Sequence
  Description of Combined DNA/RNA Molecule Synthetic oligonucleotide
ccugcugcuaugccucauctt

SEQ ID NO: 204 - DNA, 21 bp, Artificial Sequence
  Description of Combined DNA/RNA Molecule Synthetic oligonucleotide
uuugcugguccuuuccaaatt

SEQ ID NO: 205 - DNA, 21 bp, Artificial Sequence
  Description of Combined DNA/RNA Molecule Synthetic oligonucleotide
uuuggaaaggaccagcaaatt

SEQ ID NO: 206 - DNA, 21 bp, Artificial Sequence
  Description of Combined DNA/RNA Molecule Synthetic oligonucleotide
uuugcugguccuuuccaaatt

SEQ ID NO: 207 - DNA, 21 bp, Artificial Sequence
  Description of Combined DNA/RNA Molecule Synthetic oligonucleotide
uuuggaaaggaccagcaaatt

SEQ ID NO: 208 - DNA, 21 bp, Artificial Sequence
  Description of Combined DNA/RNA Molecule Synthetic oligonucleotide
cuggggcuucccgggacuctt

SEQ ID NO: 209 - DNA, 21 bp, Artificial Sequence
  Description of Combined DNA/RNA Molecule Synthetic oligonucleotide
gagucccgggaagccccagtt

SEQ ID NO: 210 - DNA, 21 bp, Artificial Sequence
  Description of Combined DNA/RNA Molecule Synthetic oligonucleotide
uguacaaguagguuccuuutt

SEQ ID NO: 211 - DNA, 21 bp, Artificial Sequence
  Description of Combined DNA/RNA Molecule Synthetic oligonucleotide
aaaggaaccuacuuguacatt

SEQ ID NO: 212 - DNA, 21 bp, Artificial Sequence
  Description of Combined DNA/RNA Molecule Synthetic oligonucleotide
cuggcagcuugucagggugtt

SEQ ID NO: 213 - DNA, 21 bp, Artificial Sequence
  Description of Combined DNA/RNA Molecule Synthetic oligonucleotide
cacccugacaagcugccagtt

SEQ ID NO: 214 - DNA, 21 bp, Artificial Sequence
  Description of Combined DNA/RNA Molecule Synthetic oligonucleotide
ccgaucacuccaaagugcatt

SEQ ID NO: 215 - DNA, 21 bp, Artificial Sequence
  Description of Combined DNA/RNA Molecule Synthetic oligonucleotide
ugcacuuuggagugaucggtt

SEQ ID NO: 216 - DNA, 21 bp, Artificial Sequence
  Description of Combined DNA/RNA Molecule Synthetic oligonucleotide
aaggagaagaggcugaggatt

SEQ ID NO: 217 - DNA, 21 bp, Artificial Sequence
  Description of Combined DNA/RNA Molecule Synthetic oligonucleotide
uccucagccucuucuccuutt

SEQ ID NO: 218 - DNA, 21 bp, Artificial Sequence
  Description of Combined DNA/RNA Molecule Synthetic oligonucleotide
uaagcuuggguuccgacccta

SEQ ID NO: 219 - DNA, 21 bp, Artificial Sequence
  Description of Combined DNA/RNA Molecule Synthetic oligonucleotide
gggucggaacccaagcuuatt

SEQ ID NO: 220 - DNA, 13 bp, Artificial Sequence
  Description of Combined DNA/RNA Molecule Synthetic oligonucleotide
ggaaaanaaaagg

SEQ ID NO: 221 - DNA, 13 bp, Artificial Sequence
  Description of Combined DNA/RNA Molecule Synthetic oligonucleotide
ccttttnttttcc

SEQ ID NO: 222 - DNA, 12 bp, Artificial Sequence
  Description of Artificial Sequence Synthetic oligonucleotide
gggaannttccc

SEQ ID NO: 223 - DNA, 21 bp, Artificial Sequence
  Description of Combined DNA/RNA Molecule Synthetic oligonucleotide
ccuacagagaacugcgguutt

SEQ ID NO: 224 - DNA, 21 bp, Artificial Sequence
  Description of Combined DNA/RNA Molecule Synthetic oligonucleotide
aaccgcaguucucuguaggtt

SEQ ID NO: 225 - DNA, 21 bp, Artificial Sequence
  Description of Combined DNA/RNA Molecule Synthetic oligonucleotide
ccuacagagaacugcgguutt

SEQ ID NO: 226 - DNA, 21 bp, Artificial Sequence
  Description of Combined DNA/RNA Molecule Synthetic oligonucleotide
aaccgcaguucucuguaggtt

SEQ ID NO: 227 - DNA, 21 bp, Artificial Sequence
  Description of Combined DNA/RNA Molecule Synthetic oligonucleotide
ccuacagagaacugcgguutt

SEQ ID NO: 228 - DNA, 21 bp, Artificial Sequence
  Description of Combined DNA/RNA Molecule Synthetic oligonucleotide
aaccgcaguucucuguaggtt

SEQ ID NO: 229 - DNA, 21 bp, Artificial Sequence
  Description of Combined DNA/RNA Molecule Synthetic oligonucleotide
ccuacagagaacugcgguutt

SEQ ID NO: 230 - DNA, 21 bp, Artificial Sequence
  Description of Combined DNA/RNA Molecule Synthetic oligonucleotide
aaccgcaguucucuguaggtt

SEQ ID NO: 231 - DNA, 21 bp, Artificial Sequence
  Description of Combined DNA/RNA Molecule Synthetic oligonucleotide
ccuacagagaacugcgguutt

SEQ ID NO: 232 - DNA, 21 bp, Artificial Sequence
  Description of Combined DNA/RNA Molecule Synthetic oligonucleotide
aaccgcaguucucuguaggtt

SEQ ID NO: 233 - DNA, 21 bp, Artificial Sequence
  Description of Combined DNA/RNA Molecule Synthetic oligonucleotide
ccuacagagaacugcgguutt

SEQ ID NO: 234 - DNA, 21 bp, Artificial Sequence
  Description of Combined DNA/RNA Molecule Synthetic oligonucleotide
aaccgcaguucucuguaggtt

SEQ ID NO: 235 - DNA, 21 bp, Artificial Sequence
  Description of Combined DNA/RNA Molecule Synthetic oligonucleotide
ccuacagagaacugcgguutt

SEQ ID NO: 236 - DNA, 21 bp, Artificial Sequence
  Description of Combined DNA/RNA Molecule Synthetic oligonucleotide
aaccgcaguucucuguaggtt

SEQ ID NO: 237 - DNA, 21 bp, Artificial Sequence
  Description of Combined DNA/RNA Molecule Synthetic oligonucleotide
ccuacagagaacugcgguutt

SEQ ID NO: 238 - DNA, 21 bp, Artificial Sequence
  Description of Combined DNA/RNA Molecule Synthetic oligonucleotide
aaccgcaguucucuguaggtt

SEQ ID NO: 239 - DNA, 21 bp, Artificial Sequence
  Description of Combined DNA/RNA Molecule Synthetic oligonucleotide
ccuacagagaacugcgguutt

SEQ ID NO: 240 - DNA, 21 bp, Artificial Sequence
  Description of Combined DNA/RNA Molecule Synthetic oligonucleotide
aaccgcaguucucuguaggtt

SEQ ID NO: 241 - DNA, 21 bp, Artificial Sequence
  Description of Combined DNA/RNA Molecule Synthetic oligonucleotide
ccuacagagaacugcgguutt

SEQ ID NO: 242 - DNA, 21 bp, Artificial Sequence
  Description of Combined DNA/RNA Molecule Synthetic oligonucleotide
aaccgcaguucucuguaggtt

SEQ ID NO: 243 - DNA, 21 bp, Artificial Sequence
  Description of Combined DNA/RNA Molecule Synthetic oligonucleotide
auugaccaccagguuucugtt

SEQ ID NO: 244 - DNA, 21 bp, Artificial Sequence
  Description of Combined DNA/RNA Molecule Synthetic oligonucleotide
cagaaaccugguggucaautt

SEQ ID NO: 245 - DNA, 21 bp, Artificial Sequence
  Description of Combined DNA/RNA Molecule Synthetic oligonucleotide
auugaccaccagguuucugtt

SEQ ID NO: 246 - DNA, 21 bp, Artificial Sequence
  Description of Combined DNA/RNA Molecule Synthetic oligonucleotide
cagaaaccugguggucaautt

SEQ ID NO: 247 - DNA, 21 bp, Artificial Sequence
  Description of Combined DNA/RNA Molecule Synthetic oligonucleotide
auugaccaccagguuucugtt

SEQ ID NO: 248 - DNA, 21 bp, Artificial Sequence
  Description of Combined DNA/RNA Molecule Synthetic oligonucleotide
cagaaaccugguggucaautt

SEQ ID NO: 249 - DNA, 21 bp, Artificial Sequence
  Description of Combined DNA/RNA Molecule Synthetic oligonucleotide
auugaccaccagguuucugtt

SEQ ID NO: 250 - DNA, 21 bp, Artificial Sequence
  Description of Combined DNA/RNA Molecule Synthetic oligonucleotide
cagaaaccugguggucaautt

SEQ ID NO: 251 - DNA, 21 bp, Artificial Sequence
  Description of Combined DNA/RNA Molecule Synthetic oligonucleotide
atugaccaccagguutcugtt

SEQ ID NO: 252 - DNA, 21 bp, Artificial Sequence
  Description of Combined DNA/RNA Molecule Synthetic oligonucleotide
cagaaaccugguggucaautt

SEQ ID NO: 253 - DNA, 21 bp, Artificial Sequence
  Description of Combined DNA/RNA Molecule Synthetic oligonucleotide
atugaccaccagguutcugtt

SEQ ID NO: 254 - DNA, 21 bp, Artificial Sequence
  Description of Combined DNA/RNA Molecule Synthetic oligonucleotide
cagaaaccugguggucaautt

SEQ ID NO: 255 - DNA, 21 bp, Artificial Sequence
  Description of Combined DNA/RNA Molecule Synthetic oligonucleotide
auugaccaccagguutcugtt

SEQ ID NO: 256 - DNA, 21 bp, Artificial Sequence
  Description of Combined DNA/RNA Molecule Synthetic oligonucleotide
cagaaaccugguggucaautt

SEQ ID NO: 257 - DNA, 21 bp, Artificial Sequence
  Description of Combined DNA/RNA Molecule Synthetic oligonucleotide
auugaccaccagguuucugtt

SEQ ID NO: 258 - DNA, 21 bp, Artificial Sequence
  Description of Combined DNA/RNA Molecule Synthetic oligonucleotide
cagaaaccugguggucaautt

SEQ ID NO: 259 - DNA, 21 bp, Artificial Sequence
  Description of Combined DNA/RNA Molecule Synthetic oligonucleotide
auugaccaccagguuucugtt

SEQ ID NO: 260 - DNA, 21 bp, Artificial Sequence
  Description of Combined DNA/RNA Molecule Synthetic oligonucleotide
cagaaaccugguggucaautt

SEQ ID NO: 261 - DNA, 21 bp, Artificial Sequence
  Description of Combined DNA/RNA Molecule Synthetic oligonucleotide
auugaccaccagguuucugtt

SEQ ID NO: 262 - DNA, 21 bp, Artificial Sequence
  Description of Combined DNA/RNA Molecule Synthetic oligonucleotide
cagaaaccugguggucaautt

SEQ ID NO: 263 - DNA, 21 bp, Artificial Sequence
  Description of Combined DNA/RNA Molecule Synthetic oligonucleotide
auugaccaccagguuucugtt

SEQ ID NO: 264 - DNA, 21 bp, Artificial Sequence
  Description of Combined DNA/RNA Molecule Synthetic oligonucleotide
cagaaaccugguggucaautt

SEQ ID NO: 265 - DNA, 21 bp, Artificial Sequence
  Description of Combined DNA/RNA Molecule Synthetic oligonucleotide
auugaccaccagguuucugtt

SEQ ID NO: 266 - DNA, 21 bp, Artificial Sequence
  Description of Combined DNA/RNA Molecule Synthetic oligonucleotide
cagaaaccugguggucaautt

SEQ ID NO: 267 - DNA, 21 bp, Artificial Sequence
  Description of Combined DNA/RNA Molecule Synthetic oligonucleotide
aaucuuauauuugauccaatt

SEQ ID NO: 268 - DNA, 21 bp, Artificial Sequence
  Description of Combined DNA/RNA Molecule Synthetic oligonucleotide
uuggaucaaauauaagauutt

SEQ ID NO: 269 - DNA, 21 bp, Artificial Sequence
  Description of Combined DNA/RNA Molecule Synthetic oligonucleotide
aaucuuauauuugauccaatt

SEQ ID NO: 270 - DNA, 21 bp, Artificial Sequence
  Description of Combined DNA/RNA Molecule Synthetic oligonucleotide
auugguauucagugugaugtt

SEQ ID NO: 271 - DNA, 21 bp, Artificial Sequence
  Description of Combined DNA/RNA Molecule Synthetic oligonucleotide
caucacacugaauaccaautt

SEQ ID NO: 272 - DNA, 21 bp, Artificial Sequence
  Description of Combined DNA/RNA Molecule Synthetic oligonucleotide
uuggaucaaauauaagauutt

SEQ ID NO: 273 - DNA, 21 bp, Artificial Sequence
  Description of Combined DNA/RNA Molecule Synthetic oligonucleotide
caucacacugaauaccaautt

SEQ ID NO: 274 - DNA, 21 bp, Artificial Sequence
  Description of Combined DNA/RNA Molecule Synthetic oligonucleotide
uuggaucaaauauaagauutt

SEQ ID NO: 275 - DNA, 21 bp, Artificial Sequence
  Description of Combined DNA/RNA Molecule Synthetic oligonucleotide
caucacacugaauaccaautt

SEQ ID NO: 276 - DNA, 21 bp, Artificial Sequence
  Description of Combined DNA/RNA Molecule Synthetic oligonucleotide
auugguauucagugugaugtt

SEQ ID NO: 277 - DNA, 21 bp, Artificial Sequence
  Description of Combined DNA/RNA Molecule Synthetic oligonucleotide
aaucuuauauuugauccaatt

SEQ ID NO: 278 - DNA, 21 bp, Artificial Sequence
  Description of Combined DNA/RNA Molecule Synthetic oligonucleotide
uuggaucaaauauaagauutt

SEQ ID NO: 279 - DNA, 21 bp, Artificial Sequence
  Description of Combined DNA/RNA Molecule Synthetic oligonucleotide
aaucuuauauuugauccaatt

SEQ ID NO: 280 - DNA, 20 bp, Artificial Sequence
  Description of Combined DNA/RNA Molecule Synthetic oligonucleotide
uuggaucaaauauaagauut

SEQ ID NO: 281 - DNA, 21 bp, Artificial Sequence
  Description of Combined DNA/RNA Molecule Synthetic oligonucleotide
aaucuuauauuugauccaatt

SEQ ID NO: 282 - DNA, 21 bp, Artificial Sequence
  Description of Combined DNA/RNA Molecule Synthetic oligonucleotide
auugguauucagugugaugtt

SEQ ID NO: 283 - DNA, 21 bp, Artificial Sequence
  Description of Combined DNA/RNA Molecule Synthetic oligonucleotide
aaucuuauauuugauccaatt

SEQ ID NO: 284 - DNA, 21 bp, Artificial Sequence
  Description of Combined DNA/RNA Molecule Synthetic oligonucleotide
uuggaucaaauauaagauutt

SEQ ID NO: 285 - DNA, 21 bp, Artificial Sequence
  Description of Combined DNA/RNA Molecule Synthetic oligonucleotide
aaucuuauauuugauccaatt

SEQ ID NO: 286 - DNA, 21 bp, Artificial Sequence
  Description of Combined DNA/RNA Molecule Synthetic oligonucleotide
uuggaucaaauauaagauutt

SEQ ID NO: 287 - DNA, 21 bp, Artificial Sequence
  Description of Combined DNA/RNA Molecule Synthetic oligonucleotide
aaucuuauauuugauccaatt

SEQ ID NO: 288 - DNA, 21 bp, Artificial Sequence
  Description of Combined DNA/RNA Molecule Synthetic oligonucleotide
uuggaucaaauauaagauutt

(19) United States
(12) Patent Application Publication
(10) Pub. No.: US 2010/0015708 A1
(43) Pub. Date: Jan. 21, 2010

(54) RIBONUCLEIC ACIDS WITH NON-STANDARD BASES AND USES THEREOF

(75) Inventors: Steven C. Quay, Woodinville, WA (US); Narendra K. Vaish, Kirkland, WA (US)

Correspondence Address: NASTECH PHARMACEUTICAL COMPANY INC;MDRNA, Inc., 3830 MONTE VILLA PARKWAY, BOTHELL, WA, 98021-7266, US

(73) Assignee: MDRNA, INC., Bothell, WA (US)

(21) Appl. No.: 12/486,889

(22) Filed: Jun. 18, 2009

Related U.S. Application Data
(60) Provisional application No. 61/073,633, filed on Jun. 18, 2008.

(51) Int. Cl. C12N 5/06; C07H 21/02

(52) U.S. Cl. 435/375; 536/23.1

(57) ABSTRACT

The present disclosure provides a ribonucleic acid comprising a double-stranded region having at least one base pair comprising a 5-methyluridine base paired with a 2,6-diaminopurine and methods for preparing the same. Also provided are methods for treating or preventing a disease or disorder by inducing RNAi.

### TECHNICAL FIELD

The present disclosure relates generally to the nucleic acid compounds and their uses in treating various diseases or disorders, by means of RNA interference (RNAi) and, more specifically, to double-stranded ribonucleic acids (dsRNAs) that decrease expression of a target nucleic acid by RNAi, which dsRNAs have a double-stranded region of about 10 to about 40 base pairs and at least one base pair comprising a 5-methyluridine base paired with a 2,6-diaminopurine in the double-stranded region of the dsRNA.

### BACKGROUND

Recent developments in the area of RNA interference (RNAi) show that this technology is emerging as a promising therapy for modifying expression of specific genes in plant and animal cells, which will be useful for treating a wide range of diseases and disorders caused by or associated with inappropriate gene expression. In particular, RNAi will be useful for treating diseases in which reducing or inhibiting gene expression is beneficial.

RNAi refers to the process of sequence-specific post-transcriptional gene silencing (also referred to as quelling) mediated by small double-stranded RNAs. See Fire et al., Nature 391:806, 1998, and Hamilton et al., Science 286:950-951, 1999. The presence of long double-stranded ribonucleic acids (dsRNAs) in cells stimulates the activity of a ribonuclease III enzyme referred to as Dicer, which processes the dsRNA into short pieces known as short interfering RNAs (siRNAs) (Hamilton et al., supra; Berstein et al., Nature 409:363, 2001). Short interfering RNAs derived from Dicer activity are generally about 19 to 23 nucleotides in length (Hamilton et al., supra; Elbashir et al., Genes Dev. 15:188, 2001). The dsRNAs are then incorporated into a multicomponent nuclease complex known as RNA-induced silencing complex (RISC), which mediates cleavage of a target single-stranded RNA (e.g., mRNA) having sequence complementary to the antisense strand of the dsRNA duplex. Cleavage of the target RNA takes place in the middle of the region complementary to the antisense strand of the dsRNA duplex (Elbashir et al., Genes Dev. 15:188, 2001).

There continues to be a need for effective therapeutic modalities useful for treating or preventing diseases or disorders in which reduced gene expression (gene silencing) would be beneficial. The present disclosure meets such needs, and further provides other related advantages.

### DETAILED DESCRIPTION

The instant disclosure provides ribonucleic acid compounds and a method for activating target gene-specific RNA interference (RNAi) by administering a double-stranded ribonucleic acid (dsRNA) to a cell expressing a target gene in an amount sufficient to reduce expression of the target gene by RNAi. The ribonucleic acid compounds include double-stranded ribonucleic acids (dsRNAs) having a double-stranded region of about 10 to about 40 base pairs and at least one base pair comprising a 5-methyluridine base paired with a 2,6-diaminopurine in the double-stranded region of the dsRNA. These dsRNAs may have reduced or minimal off-target effects, have minimal or no activation of an interferon response in target cells, have increased potency, and/or have improved stability. Also provided herein are methods of using such dsRNA to treat or prevent various diseases or disorders, including hyperproliferative disorders (e.g., cancer), inflammatory conditions, neurological disorders, cardiac conditions, respiratory diseases, or autoimmune disorders.

Prior to setting forth this disclosure in more detail, it may be helpful to an understanding thereof to provide definitions of certain terms to be used herein.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, “about” or “consisting essentially of” mean ±20% of the indicated range, value, or structure, unless otherwise indicated. As used herein, the terms “include” and “comprise” are used synonymously. It should be understood that the terms “a” and “an” as used herein refer to “one or more” of the enumerated components. The use of the alternative (e.g., “or”) should be understood to mean either one, both, or any combination thereof of the alternatives.

As used herein, “complementary” refers to a nucleic acid molecule that can form hydrogen bond(s) with another nucleic acid molecule by either traditional Watson-Crick base pairing or other non-traditional types of pairing (e.g., Hoogsteen or reversed Hoogsteen hydrogen bonding) between complementary nucleosides or nucleotides. In reference to the nucleic molecules of the present disclosure, the binding free energy for a nucleic acid molecule with its complementary sequence is sufficient to allow the relevant function of the nucleic acid molecule to proceed, e.g., RNAi activity, and there is a sufficient degree of complementarity to avoid non-specific binding of the nucleic acid molecule (e.g., dsRNA) to non-target sequences under conditions in which specific binding is desired, i.e., under physiological conditions in the case of in vivo assays or therapeutic treatment, or in the case of in vitro assays, under conditions in which the assays are performed (e.g., hybridization assays). Determination of binding free energies for nucleic acid molecules is well known in the art (see e.g., Turner et al., CSH Symp. Quant. Biol. LII:123-133, 1987; Frier et al., Proc. Nat. Acad. Sci. USA 83:9373-77, 1986; Turner et al., J. Am. Chem. Soc. 109:3783-3785, 1987). Thus, “complementary” (or “specifically hybridizable”) are terms that indicate a sufficient degree of complementarity or precise pairing such that stable and specific binding occurs between a nucleic acid molecule (e.g., dsRNA) and a DNA or RNA target. It is understood in the art that a nucleic acid molecule need not be 100% complementary to a target nucleic acid sequence to be specifically hybridizable. That is, two or more nucleic acid molecules may be less than fully complementary and is indicated by a percentage of contiguous residues in a nucleic acid molecule that can form hydrogen bonds with a second nucleic acid molecule. For example, if a first nucleic acid molecule has 10 nucleotides and a second nucleic acid molecule has 10 nucleotides, then base pairing of 5, 6, 7, 8, 9, or 10 nucleotides between the first and second nucleic acid molecules represents 50%, 60%, 70%, 80%, 90%, and 100% complementarity, respectively. “Perfectly” or “fully” complementary nucleic acid molecules means those in which all the contiguous residues of a first nucleic acid molecule will hydrogen bond with the same number of contiguous residues in a second nucleic acid molecule, wherein the nucleic acid molecules either both have the same number of nucleotides (i.e., have the same length) or the two molecules have different lengths.

By “ribonucleic acid” or “RNA” is meant a nucleic acid molecule comprising at least one ribonucleotide molecule. As used herein, “ribonucleotide” refers to a nucleotide with a hydroxyl group at the 2′-position of a β-D-ribofuranose moiety. The term RNA includes double-stranded (ds) RNA, single-stranded (ss) RNA, isolated RNA (such as partially purified RNA, essentially pure RNA, synthetic RNA, recombinantly produced RNA), altered RNA (which differs from naturally occurring RNA by the addition, deletion, substitution or alteration of one or more nucleotides), or any combination thereof. For example, such altered RNA can include addition of non-nucleoside material, such as at one or both ends of an RNA molecule, internally at one or more nucleosides of the RNA, or any combination thereof. Nucleosides in RNA molecules of the instant disclosure can also comprise non-standard nucleosides, such as naturally occurring nucleosides, non-naturally occurring nucleosides, chemically-modified nucleosides, deoxynucleosides, or any combination thereof. These altered RNAs may be referred to as analogs or analogs of RNA containing standard nucleosides (i.e., standard nucleosides, as used herein, are considered to be adenosine, cytidine, guanosine, thymidine, and uridine).

The term “dsRNA” as used herein refers to any nucleic acid molecule comprising at least one ribonucleotide molecule and capable of inhibiting or down regulating gene expression, for example, by mediating RNA interference (“RNAi”) or gene silencing in a sequence-specific manner. The dsRNAs of the instant disclosure may be suitable substrates for Dicer or for association with RISC to mediate gene silencing by RNAi. Illustrative dsRNA molecules substituted or modified as described herein and useful in the methods of this disclosure can be found in, for example, U.S. patent application Ser. No. 11/681,725; U.S. Pat. Nos. 7,022,828 and 7,034,009; U.S. Patent Application Publication No. 2004/01381; PCT Application Publication No. WO 03/070897. One or both stands of the dsRNA can further comprise a terminal phosphate group, such as a 5′-phosphate or a 5′,3′-diphosphate. As used herein, dsRNA molecules can further include additional substitutions, chemically-modified nucleosides, or non-nucleotides. In certain embodiments, dsRNA molecules can comprise ribonucleotides up to about 100% of the nucleotide positions, which can be standard or non-standard nucleotides. As used herein, the term dsRNA is meant to be equivalent to other terms used to describe nucleic acid molecules that are capable of mediating sequence specific RNAi, for example, short interfering nucleic acid (siNA), siRNA, micro-RNA (miRNA), short hairpin RNA (shRNA), short interfering oligonucleotide, short interfering substituted oligonucleotide, short interfering modified oligonucleotide, chemically-modified dsRNA, post-transcriptional gene silencing RNA (ptgsRNA), dsRNA having at least one base pair comprising a 5-methyluridine base paired with a 2,6-diaminopurine (drtRNA), or the like. In addition, as used herein, the term “RNAi” is meant to be equivalent to other terms used to describe sequence specific RNA interference, such as post-transcriptional gene silencing, translational inhibition, or epigenetics. For example, dsRNA molecules of this disclosure can be used to epigenetically silence genes at the post-transcriptional level or the pre-transcriptional level or any combination thereof.

In one aspect, a dsRNA comprises two separate oligonucleotides, comprising a first strand (antisense) and a second strand (sense), wherein the antisense and sense strands are self-complementary (i.e., each strand comprises a nucleotide sequence that is complementary to a nucleotide sequence in the other strand and the two separate strands form a duplex or double-stranded structure, for example, wherein the double-stranded region is about 10 to about 29 base pairs or about 10, 11, 12, 13, 14, 15, 16, 17, 18, 29, 20, 21, 22, 23, 24, 25, 26, 27, 28, or 29 base pairs, or about 29 to about 40 base pairs or 29, 30, 31, 32, 33, 34, 35, 36, 37, 38, 39, or 40 base pairs); the antisense strand comprises a nucleotide sequence that is complementary to a nucleotide sequence in a target nucleic acid molecule or a portion thereof (e.g., a sequence set forth in any one of the sequences set forth in the accession numbers of Table A); and the sense strand comprises a nucleotide sequence corresponding (i.e., is homologous) to the target nucleic acid sequence or a portion thereof (e.g., a sense strand of about 10 to about 29 nucleotides or about 10, 11, 12, 13, 14, 15, 16, 17, 18, 29, 20, 21, 22, 23, 24, 25, 26, 27, 28, or 29 nucleotides, or about 29 to about 40 nucleotides or 29, 30, 31, 32, 33, 34, 35, 36, 37, 38, 39, or 40 nucleotides corresponds to the target nucleic acid or a portion thereof).

In another aspect, the dsRNA is assembled from a single oligonucleotide in which the self-complementary sense and antisense strands of the dsRNA are linked by together by a nucleic acid based-linker or a non-nucleic acid-based linker. In certain embodiments, the first (antisense) and second (sense) strands of the dsRNA molecule are covalently linked by a nucleotide or non-nucleotide linker known in the art.

In still another aspect, dsRNA molecules described herein comprise three or more strands such as, for example, an A strand, B1 strand, and B2 strand (which can form a dsRNA of A:B1B2), wherein the B1 and B2 strands are complementary to, and form base pairs (bp) with, non-overlapping regions of the A strand. The double-stranded region formed by the annealing of the B1 and A strands is distinct from and non-overlapping with the double-stranded region formed by the annealing of the B2 and A strands. In certain embodiments, the A:B1 duplex is separated from the A:B2 duplex by a “gap” resulting from at least one unpaired nucleotide in the A strand that is positioned between the A:B1 duplex and the A:B2 duplex and that is distinct from any one or more unpaired nucleotide at the 3′-end of one or more of the A, B1, or B2 strands. In another embodiment, the A:B1 duplex is separated from the A:B2 duplex by a “nick” between the A:B1 duplex and the A:B2 duplex. In one embodiment, A:B1B2, in sum, is a dsRNA having a double-stranded region ranging from about 10 base pairs to about 40 base pairs (or about 10, 11, 12, 13, 14, 15, 16, 17, 18, 29, 20, 21, 22, 23, 24, 25, 26, 27, 28, 29, 30, 31, 32, 33, 34, 35, 36, 37, 38, 39, or 40 base pairs) and having a nick or a gap. In certain embodiments, A corresponds to a sense strand, while B1 and B2 together correspond to an antisense strand (e.g., complementary to a portion of any one of the sequences set forth in the accession numbers of Table A), wherein A is about 10 to about 40 nucleotides in length (or about 10, 11, 12, 13, 14, 15, 16, 17, 18, 29, 20, 21, 22, 23, 24, 25, 26, 27, 28, 29, 30, 31, 32, 33, 34, 35, 36, 37, 38, 39, or 40 nucleotides in length), and B1 and B2 are each, individually, about 5 to about 20 nucleotides (or about 5, 6, 7, 8, 9, 10, 11, 12, 13, 14, 15, 16, 17, 18, 19, or 20 nucleotides) and the combined length of the B1 strand and the B2 strand is between about 10 nucleotides and about 40 nucleotides (or about 10, 11, 12, 13, 14, 15, 16, 17, 18, 29, 20, 21, 22, 23, 24, 25, 26, 27, 28, 29, 30, 31, 32, 33, 34, 35, 36, 37, 38, 39, or 40 nucleotides).

The term “large double-stranded (ds) RNA” refers to any double-stranded RNA having a size greater than about 40 bp to about 100 bp or more, particularly up to about 300 bp to about 500 bp. The sequence of a large dsRNA may represent a segment of an mRNA or an entire mRNA. A double-stranded structure or region, or duplex or duplex region may be formed by self-complementary nucleic acid molecule, such as occurs in a hairpin or microRNA, or by annealing of two or more distinct complementary nucleic acid molecule strands.

A dsRNA or large dsRNA may include substitution or modification in which the substitution or modification may be in a phosphate backbone bond, a sugar, or a nucleoside. Such nucleoside substitutions can include natural non-standard nucleosides (e.g., 5-methyluridine, 5-methylcytidine or 2,6-diaminopurine), and such backbone, sugar, or nucleoside modifications can include an alkyl or heteroatom substitution or addition, such as a methyl, alkoxyalkyl, halogen, nitrogen or sulfur, or any other modification known in the art.

As used herein, “target nucleic acid” refers to any nucleic acid sequence whose expression or activity is to be altered (e.g., a human gene). The target nucleic acid can be DNA, RNA, or analogs thereof, and includes single, double, and multi-stranded forms. By “target site” or “target sequence” is meant a sequence within a target nucleic acid (e.g., mRNA of a human gene) that is “targeted” for cleavage by RNAi and mediated by a dsRNA construct of this disclosure containing a sequence within the antisense strand that is complementary to the target site or sequence.

The dsRNAs of this disclosure may be targeted to various genes. Examples of human genes suitable as targets include TNF, FLT1, the VEGF family, the ERBB family, the PDGFR family, BCR-ABL, and the MAPK family, among others. Examples of human genes suitable as targets and nucleic acid sequences thereto include those disclosed in PCT/US08/55333, PCT/US08/55339, PCT/US08/55340, PCT/US08/55341, PCT/US08/55350, PCT/US08/55353, PCT/US08/55356, PCT/US08/55357, PCT/US08/55360, PCT/US08/55362, PCT/US08/55365, PCT/US08/55366, PCT/US08/55369, PCT/US08/55370, PCT/US08/55371, PCT/US08/55372, PCT/US08/55373, PCT/US08/55374, PCT/US08/55375, PCT/US08/55376, PCT/US08/55377, PCT/US08/55378, PCT/US08/55380, PCT/US08/55381, PCT/US08/55382, PCT/US08/55383, PCT/US08/55385, PCT/US08/55386, PCT/US08/55505, PCT/US08/555 11, PCT/US08/55515, PCT/US08/55516, PCT/US08/55519, PCT/US08/55524, PCT/US08/55526, PCT/US08/55527, PCT/US08/55532, PCT/US08/55533, PCT/US08/55542, PCT/US08/55548, PCT/US08/55550, PCT/US08/55551, PCT/US08/55554, PCT/US08/55556, PCT/US08/55560, PCT/US08/55563, PCT/US08/55597, PCT/US08/55599, PCT/US08/55601, PCT/US08/55603, PCT/US08/55604, PCT/US08/55606, PCT/US08/55608, PCT/US08/5561 1, PCT/US08/55612, PCT/US08/55615, PCT/US08/55618, PCT/US08/55622, PCT/US08/55625, PCT/US08/55627, PCT/US08/55631, PCT/US08/55635, PCT/US08/55644, PCT/US08/55649, PCT/US08/55651, PCT/US08/55662, PCT/US08/55672, PCT/US08/55676, PCT/US08/55678, PCT/US08/55695, PCT/US08/55697, PCT/US08/55698, PCT/US08/55701, PCT/US08/55704, PCT/US08/55708, PCT/US08/55709, and PCT/US08/55711, all hereby incorporated by reference.

A dsRNA of this disclosure to be delivered may have a nucleotide sequence that is complementary to a region of a nucleotide sequence of a viral gene. For example, some compositions and methods of this invention are useful to regulate expression of the viral genome of an influenza virus. In some embodiments, this invention provides compositions and methods for modulating expression and infectious activity of an influenza by RNA interference. Expression and/or activity of an influenza virus can be modulated by delivering to a cell, for example, a short interfering RNA molecule having a sequence that is complementary to a region of a RNA polymerase subunit of an influenza virus. Examples of RNAs targeted to an influenza virus are given in U.S. Patent Publication No. 20070213293 A1 hereby incorporated by reference.

As used herein, “off-target effect” or “off-target profile” means a dsRNA specific for a target gene or nucleic acid sequence that is capable of binding to one or more non-target gene messages resulting in non-specific inhibition (or activation) of non-target (i.e., off-target) genes in addition to specific inhibition of a target (i.e., homologous or cognate) gene expression in a cell or other biological sample. For example, an off-target effect can be quantified by using, for example, a DNA microarray to determine the number of non-target genes having an expression level that are altered by about 2-fold or more in the presence of a candidate dsRNA or analog thereof specific for a target gene. A “minimal off-target effect” means that expression of about 15 or fewer non-target genes (i.e., 0 to about 15) or less than about 1% of non-target genes are altered by about 2-fold or more in the presence of a target gene-specific dsRNA or analog thereof as compared to in the absence of the dsRNA or analog thereof. Also, a “minimal off-target effect” means that the off-target effect of a dsRNA having at least one base pair comprising a 5-methyluridine base paired with a 2,6-diaminopurine, optionally having at least one nucleotide modified at the 2′-position, is reduced by at least about 70% or more as compared to the dsRNA without having at least one base pair comprising a 5-methyluridine base paired with a 2,6-diaminopurine and unmodified dsRNA.

By “sense region” or “sense strand” is meant a nucleotide sequence of a dsRNA molecule having complementarity to an antisense region of the dsRNA molecule. In addition, the sense region of a dsRNA molecule comprises a nucleic acid sequence having homology or identity to a target sequence. By “antisense region” or “antisense strand” is meant a nucleotide sequence of a dsRNA molecule having complementarity to a target nucleic acid sequence. In addition, the antisense region of a dsRNA molecule can optionally comprise a nucleic acid sequence having complementarity to a sense region of the dsRNA molecule.

“Analog” as used herein refers to a compound that is structurally similar to a parent compound (e.g., a nucleic acid molecule), but differs slightly in composition (e.g., one atom or functional group is different, added, or removed). The analog may or may not have different chemical or physical properties than the original compound and may or may not have improved biological or chemical activity. For example, the analog may be more hydrophilic or it may have altered activity as compared to a parent compound. The analog may mimic the chemical or biological activity of the parent compound (i.e., it may have similar or identical activity), or, in some cases, may have increased or decreased activity. The analog may be a naturally or non-naturally occurring (e.g., chemically-modified or recombinant) variant of the original compound. An example of an RNA analog is an RNA molecule having a non-standard nucleotide, such as 5-methyuridine or 5-methylcytidine, which may impart certain desirable properties (e.g., improve stability, bioavailability, minimize off-target effects or interferon response).

The term “pyrimidine” as used herein refers to conventional pyrimidine bases, including standard pyrimidine bases uracil and cytosine. In addition, the term pyrimidine is contemplated to embrace natural non-standard pyrimidine bases or acids, such as 5-methyluracil, 4-thiouracil, pseudouracil, dihydrouracil, orotate, 5-methylcytosine, or the like, as well as a chemically-modified bases or “universal bases,” which can be used to substitute for a standard pyrimidine within nucleic acid molecules of this disclosure. Examples of pyrmidines suitable for use within a dsRNA of this disclosure include those disclosed in U.S. Pat. No. 6,846,827, hereby incorporated by reference.

The term “purine” as used herein refers to conventional purine bases, including standard purine bases adenine and guanine. In addition, the term purine is contemplated to embrace natural non-standard purine bases or acids, such as N2-methylguanine, inosine, 2,6-aminopurine, or the like, as well as a chemically-modified bases or “universal bases,” which can be used to substitute for a standard purine within nucleic acid molecules of this disclosure.

As used herein, the term “universal base” refers to nucleotide base analogs that form base pairs with each of the standard DNA/RNA bases with little discrimination between them. A universal base is thus interchangeable with all of the standard bases when substituted into an oligonucleotide duplex, for example, yielding a duplex that primes synthesis by DNA polymerase, directs incorporation of 5′-triphosphate of each of the standard nucleosides opposite the universal base when copied by a polymerase, serve as a substrate for polymerases as the 5′-triphosphate, and recognized by intracellular enzymes such that DNA containing the universal base can be cloned (see e.g., Loakes et al., J. Mol. Bio. 270:426-435, 1997). Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see e.g., Loakes, Nucleic Acids Res. 29:2437-2447, 2001).

The term “base pair” or “base paired” as used herein refers to not only the standard AT, AU or GC base pairs, but also base pairs formed between nucleotides and/or nucleotide analogs comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the 5-methyluridine and 2,6-diaminopurine where up to three hydrogen bonds are formed.

The term “non-standard base pair” as used herein refers to a base pair with a pattern of hydrogen bonds that hold the base pair together that differs from that found in an AT and GC base pair.

The term “gene” as used herein, especially in the context of “target gene” or “gene target” for RNAi, means a nucleic acid molecule that encodes an RNA, including messenger RNA (mRNA, also referred to as structural genes that encode for a polypeptide), a functional RNA (fRNA), or non-coding RNA (ncRNA), such as small temporal RNA (stRNA), microRNA (miRNA), small nuclear RNA (snRNA), short interfering RNA (siRNA), small nucleolar RNA (snRNA), ribosomal RNA (rRNA), transfer RNA (tRNA) and precursor RNAs thereof. Such non-coding RNAs can serve as target nucleic acid molecules for dsRNA mediated RNAi to alter the activity of fRNA or ncRNA involved in functional or regulatory cellular processes. A target gene can be a human gene derived from a cell, an endogenous gene, a transgene, or exogenous gene. The cell containing the target gene can be derived from or contained in any organism, for example, a plant, animal, protozoan, virus, bacterium, or fungus.

As used herein, “gene silencing” refers to partial or complete loss-of-function through targeted inhibition of gene expression in a cell, which may also be referred to as RNAi, “knockdown,” “inhibition,” “down-regulation,” or “reduction” of expression of a target gene, such as a human gene. Depending on the circumstances and the biological problem to be addressed, it may be preferable to partially reduce gene expression. Alternatively, it might be desirable to reduce gene expression as much as possible. The extent of silencing may be determined by methods described herein and as known in the art, some of which are summarized in PCT Publication No. WO 99/32619. Depending on the assay, quantification of gene expression permits detection of various amounts of inhibition that may be desired in certain embodiments of this disclosure, including prophylactic and therapeutic methods, which will be capable of knocking down target gene expression, in terms of mRNA level or protein level or activity, for example, by equal to or greater than 10%, 30%, 50%, 75% 90%, 95% or 99% of baseline (i.e., normal) or other control levels, including elevated expression levels as may be associated with particular disease states or other conditions targeted for therapy.

As used herein, “cell” is used in its usual biological sense and does not refer to an entire multicellular organism, such as, e.g., a human. The cell can be isolated, in culture, or present in an organism, e.g., a bird, plant, or mammal, such as a human, cow, sheep, ape, monkey, swine, mouse, dog, or cat. The cell can be prokaryotic (e.g., bacterial cell) or eukaryotic (e.g., mammalian or plant). The cell can be of somatic or germ line origin, totipotent or pluripotent, dividing or non-dividing. The cell can also be derived from or can comprise a gamete or embryo, a stem cell, or a fully differentiated cell.

By “subject” is meant an organism, which is a donor or recipient of explanted cells or the cells themselves. “Subject” also refers to an organism to which the nucleic acid molecules of this disclosure can be administered. In one embodiment, a subject is a mammal or mammalian cell. In another embodiment, a subject is a human or human cell.

As used herein, the term “therapeutically effective amount” means an amount of dsRNA that is sufficient, in the subject (e.g., human) to which it is administered, to treat or prevent the stated disease, disorder, or condition. For example, a therapeutically effective amount of dsRNA directed against a target gene (e.g., a sequence set forth in any one of the sequences set forth in the accession numbers of Table A), which effectively down-regulates the target gene mRNA and thereby reduces or prevents one or more target gene-associated disease, disorder, or condition. The nucleic acid molecules of the instant disclosure, individually, or in combination or in conjunction with other drugs, can be used to treat diseases or conditions discussed herein. For example, to treat a particular disease, disorder, or condition, the dsRNA molecules can be administered to a patient or can be administered to other appropriate cells evident to those skilled in the art, individually or in combination with one or more drugs, under conditions suitable for treatment.

In addition, it should be understood that the individual compounds, or groups of compounds, derived from the various combinations of the structures and substituents described herein, are disclosed by the present application to the same extent as if each compound or group of compounds was set forth individually. Thus, selection of particular structures or particular substituents is within the scope of the present disclosure. As described herein, all value ranges are inclusive over the indicated range. Thus, a range of C₁-C₄ will be understood to include the values of 1, 2, 3, and 4, such that C₁, C₂, C₃ and C₄ are included.

The term “alkyl” as used herein refers to saturated straight- or branched-chain aliphatic groups containing from 1-20 carbon atoms, preferably 1-8 carbon atoms and most preferably 1-4 carbon atoms. This definition applies as well to the alkyl portion of alkoxy, alkanoyl and aralkyl groups. The alkyl group may be substituted or unsubstituted. In certain embodiments, the alkyl is a (C₁-C₄) alkyl or methyl.

The term “cycloalkyl” as used herein refers to a saturated cyclic hydrocarbon ring system containing from 3 to 12 carbon atoms that may be optionally substituted. Exemplary embodiments include, but are not limited to, cyclopropyl, cyclobutyl, cyclopentyl and cyclohexyl. In certain embodiments, the cycloalkyl group is cyclopropyl. In another embodiment, the (cycloalkyl)alkyl groups contain from 3 to 12 carbon atoms in the cyclic portion and 1 to 6 carbon atoms in the alkyl portion. In certain embodiments, the (cycloalkyl)alkyl group is cyclopropylmethyl. The alkyl groups are optionally substituted with from one to three substituents selected from the group consisting of halogen, hydroxy and amino.

The terms “alkanoyl” and “alkanoyloxy” as used herein refer, respectively, to —C(O)-alkyl groups and —O—C(═O)— alkyl groups, each optionally containing 2 to 10 carbon atoms. Specific embodiments of alkanoyl and alkanoyloxy groups are acetyl and acetoxy, respectively.

The term “alkenyl” refers to an unsaturated branched, straight-chain or cyclic alkyl group having 2 to 15 carbon atoms and having at least one carbon-carbon double bond derived by the removal of one hydrogen atom from a single carbon atom of a parent alkene. The group may be in either the cis or trans conformation about the double bond(s). Certain embodiments include ethenyl, 1-propenyl, 2-propenyl, 1-methylethenyl, 1-butenyl, 2-butenyl, 3-butenyl, 2-methyl-2-propenyl, 1-pentenyl, 2-pentenyl, 4-pentenyl, 3-methyl-2-butenyl, 1-hexenyl, 2-hexenyl, 1-heptenyl, 2-heptenyl, 1-octenyl, 2-octenyl, 1,3-octadienyl, 2-nonenyl, 1,3-nonadienyl, 2-decenyl, etc., or the like. The alkenyl group may be substituted or unsubstituted.

The term “alkynyl” as used herein refers to an unsaturated branched, straight-chain, or cyclic alkyl group having 2 to 10 carbon atoms and having at least one carbon-carbon triple bond derived by the removal of one hydrogen atom from a single carbon atom of a parent alkyne. Exemplary alkynyls include ethynyl, 1-propynyl, 2-propynyl, 1-butynyl, 2-butynyl, 3-butynyl, 1-pentynyl, 2-pentynyl, 4-pentynyl, 1-octynyl, 6-methyl-1-heptynyl, 2-decynyl, or the like. The alkynyl group may be substituted or unsubstituted.

The term “hydroxyalkyl” alone or in combination, refers to an alkyl group as previously defined, wherein one or several hydrogen atoms, preferably one hydrogen atom has been replaced by a hydroxyl group. Examples include hydroxymethyl, hydroxyethyl and 2-hydroxyethyl.

The term “aminoalkyl” as used herein refers to the group —NRR′, where R and R′ may independently be hydrogen or (C₁-C₄) alkyl.

The term “alkylaminoalkyl” refers to an alkylamino group linked via an alkyl group (i.e., a group having the general structure -alkyl-NH-alkyl or -alkyl-N(alkyl)(alkyl)). Such groups include, but are not limited to, mono- and di-(C₁-C₈ alkyl)aminoC₁-C₈ alkyl, in which each alkyl may be the same or different.

The term “dialkylaminoalkyl” refers to alkylamino groups attached to an alkyl group. Examples include, but are not limited to, N,N-dimethylaminomethyl, N,N-dimethylaminoethyl N,N-dimethylaminopropyl, and the like. The term dialkylaminoalkyl also includes groups where the bridging alkyl moiety is optionally substituted.

The term “haloalkyl” refers to an alkyl group substituted with one or more halo groups, for example chloromethyl, 2-bromoethyl, 3-iodopropyl, trifluoromethyl, perfluoropropyl, 8-chlorononyl, or the like.

The term “carboxyalkyl” as used herein refers to the substituent —R^{z}—COOH, wherein R¹⁰ is alkylene; and carbalkoxyalkyl refers to —R¹⁰—C(═O)OR¹¹, wherein R¹⁰ and R¹¹ are alkylene and alkyl respectively. In certain embodiments, alkyl refers to a saturated straight- or branched-chain hydrocarbyl radical of 1 to 6 carbon atoms such as methyl, ethyl, n-propyl, isopropyl, n-butyl, t-butyl, n-pentyl, 2-methylpentyl, n-hexyl, and so forth. Alkylene is the same as alkyl except that the group is divalent.

The term “alkoxy” includes substituted and unsubstituted alkyl, alkenyl, and alkynyl groups covalently linked to an oxygen atom. In one embodiment, the alkoxy group contains 1 to about 10 carbon atoms. Embodiments of alkoxy groups include, but are not limited to, methoxy, ethoxy, isopropyloxy, propoxy, butoxy, and pentoxy groups. Embodiments of substituted alkoxy groups include halogenated alkoxy groups. In a further embodiment, the alkoxy groups can be substituted with groups such as alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkylamino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Exemplary halogen substituted alkoxy groups include, but are not limited to, fluoromethoxy, difluoromethoxy, trifluoromethoxy, chloromethoxy, dichloromethoxy, and trichloromethoxy.

The term “alkoxyalkyl” refers to an alkylene group substituted with an alkoxy group. For example, methoxyethyl (CH₃OCH₂CH₂—) and ethoxymethyl (CH₃CH₂OCH₂—) are both C₃ alkoxyalkyl groups.

The term “aryl” as used herein refers to monocyclic or bicyclic aromatic hydrocarbon groups having from 6 to 12 carbon atoms in the ring portion, for example, phenyl, naphthyl, biphenyl and diphenyl groups, each of which may be substituted with, for example, one to four substituents such as alkyl; substituted alkyl as defined above, halogen, trifluoromethyl, trifluoromethoxy, hydroxy, alkoxy, cycloalkyloxy, alkanoyl, alkanoyloxy, amino, alkylamino, dialkylamino, nitro, cyano, carboxy, carboxyalkyl, carbamyl, carbamoyl and aryloxy. Specific embodiments of aryl groups in accordance with the present disclosure include phenyl, substituted phenyl, naphthyl, biphenyl, and diphenyl.

The term “aroyl,” as used alone or in combination herein, refers to an aryl radical derived from an aromatic carboxylic acid, such as optionally substituted benzoic or naphthoic acids.

The term “aralkyl” as used herein refers to an aryl group bonded to the 2-pyridinyl ring or the 4-pyridinyl ring through an alkyl group, preferably one containing 1 to 10 carbon atoms. A preferred aralkyl group is benzyl.

The term “carboxy,” as used herein, represents a group of the formula —C(═O)OH or —C(═O)O—.

The term “carbonyl” as used herein refers to a group in which an oxygen atom is double-bonded to a carbon atom —C═O.

The term “trifluoromethyl” as used herein refers to —CF₃.

The term “trifluoromethoxy” as used herein refers to —OCF₃.

The term “hydroxyl” as used herein refers to —OH or —O—.

The term “nitrile” or “cyano” as used herein refers to the group —CN.

The term “nitro,” as used herein alone or in combination refers to a —NO₂ group.

The term “amino” as used herein refers to the group —NR⁹R⁹, wherein R⁹ may independently be hydrogen, alkyl, aryl, alkoxy, or heteroaryl. The term “aminoalkyl” as used herein represents a more detailed selection as compared to “amino” and refers to the group —NR′R′, wherein R′ may independently be hydrogen or (C₁-C₄) alkyl. The term “dialkylamino” refers to an amino group having two attached alkyl groups that can be the same or different.

The term “alkanoylamino” refers to alkyl, alkenyl or alkynyl groups containing the group —C(═O)— followed by —N(H)—, for example acetylamino, propanoylamino and butanoylamino and the like.

The term “carbonylamino” refers to the group —NR′—CO—CH₂—R′, wherein R′ may be independently selected from hydrogen or (C₁-C₄) alkyl.

The term “carbamoyl” as used herein refers to —O—C(O)NH₂.

The term “carbamyl” as used herein refers to a functional group in which a nitrogen atom is directly bonded to a carbonyl, i.e., as in —NR″C(═O)R″ or —C(═O)NR″R″, wherein R″ can be independently hydrogen, alkyl, substituted alkyl, alkenyl, substituted alkenyl, alkoxy, cycloalkyl, aryl, heterocyclo, or heteroaryl.

The term “alkylsulfonylamino” refers to refers to the group —NHS(O)₂R¹², wherein R¹² is alkyl.

The term “halogen” as used herein refers to bromine, chlorine, fluorine or iodine. In one embodiment, the halogen is fluorine. In another embodiment, the halogen is chlorine.

The term “heterocyclo” refers to an optionally substituted, unsaturated, partially saturated, or fully saturated, aromatic or nonaromatic cyclic group that is a 4 to 7 membered monocyclic, or 7 to 11 membered bicyclic ring system that has at least one heteroatom in at least one carbon atom-containing ring. The substituents on the heterocyclo rings may be selected from those given above for the aryl groups. Each ring of the heterocyclo group containing a heteroatom may have 1, 2, or 3 heteroatoms selected from nitrogen, oxygen and sulfur atoms. Plural heteroatoms in a given heterocyclo ring may be the same or different.

Exemplary monocyclic heterocyclo groups include pyrrolidinyl, pyrrolyl, indolyl, pyrazolyl, imidazolyl, oxazolyl, isoxazolyl, thiazolyl, furyl, tetrahydrofuryl, thienyl, piperidinyl, piperazinyl, azepinyl, pyrimidinyl, pyridazinyl, tetrahydropyranyl, morpholinyl, dioxanyl,triazinyl and triazolyl. Preferred bicyclic heterocyclo groups include benzothiazolyl, benzoxazolyl, benzothienyl, quinolinyl, tetrahydroisoquinolinyl, benzimidazolyl, benzofuryl, indazolyl, benzisothiazolyl, isoindolinyl and tetrahydroquinolinyl. In more detailed embodiments heterocyclo groups may include indolyl, imidazolyl, furyl, thienyl, thiazolyl, pyrrolidyl, pyridyl and pyrimidyl.

“Substituted” refers to a group in which one or more hydrogen atoms are each independently replaced with the same or different substituent(s). Representative substituents include —X, —R⁶, —O—, ═O, —OR, —SR , —S—, ═S, —NR R ,═NR⁶, —CX₃, —CF₃, —CN, —OCN, —SCN, —NO, —NO₂, ═N₂, —N₃, —S(═O)₂2O—, —S(═O)₂OH, —S(═O)₂R⁶, —OS(═O)₂O—, —OS(═O)₂OH, —OS(═O)₂R⁶, —P(═O)(O⁻)₂, —P(═O)(OH)(O⁻), —OP(═O)₂(O⁻), —C(—O)R⁶, —C(═S)R⁶, —C(═O)OR⁶, —C(═O)O⁻, —C(═S)OR⁶, —NR⁶—C(═O)—N(R⁶)₂, —NR⁶—C(═S)—N(R⁶)₂, and —C(═NR⁶)NR⁶R⁶, wherein each X is independently a halogen; and each R⁶ is independently hydrogen, halogen, alkyl, aryl, arylalkyl, arylaryl, arylheteroalkyl, heteroaryl, heteroarylalkyl, NR⁷R⁷, —C(═O)R⁷, and —S(═O)₂R⁷; and each R⁷ is independently hydrogen, alkyl, alkanyl, alkynyl, aryl, arylalkyl, arylheteralkyl, arylaryl, heteroaryl or heteroarylalkyl. Aryl containing substituents, whether or not having one or more substitutions, may be attached in a para (p-), meta (m-) or ortho (o-) conformation, or any combination thereof.

As used herein the term “hydrogen bond donor”, “hydrogen bond donor group”, or “donor group” means a hydrogen atom attached to a relatively electronegative atom such as nitrogen and oxygen. Non-limiting examples of a hydrogen bond donor include —SH, —OH, —NH, and —SeH.

As used herein the term “hydrogen bond acceptor”, “hydrogen bond acceptor group”, or “acceptor group” means an electronegative atom such nitrogen, oxygen, fluorine. Non-limiting examples of a hydrogen bond acceptor include —C═S, —N, and —O.

### Target Nucleic Acids and Genes

This disclosure provides compounds, compositions, and methods useful for altering expression or activity of a target gene by RNA interference (RNAi) using small nucleic acid molecules. In more detailed embodiments, this disclosure provides small nucleic acid molecules, such as short interfering nucleic acid (siNA), short interfering RNA (siRNA), double-stranded RNA (dsRNA), nicked double-stranded RNA (ndsRNA), gapped double-stranded RNA (gdsRNA), micro-RNA (mRNA), short hairpin RNA (shRNA) molecules, or any combination thereof and related compositions and methods, which are effective for altering expression of a target gene or family of genes to prevent, treat, or alleviate symptoms of a disease or disorder in a mammalian subject (e.g., human). Within these and related therapeutic compositions and methods, the use of a dsRNA of this disclosure will often improve properties of the dsRNA in comparison to the properties of native dsRNA molecules, such as reduced off-target effects, reduced interferon response, increased resistance to nuclease degradation in vivo, improved thermal stability, improved cellular uptake, increased potency, or any combination thereof.

In one embodiment, the instant disclosure provides a dsRNA useful for modulating expression of a target nucleic acid in vitro or in vivo, wherein the dsRNA comprises a double-stranded region having about 10 to about 40 base pairs (or about 10, 11, 12, 13, 14, 15, 16, 17, 18, 29, 20, 21, 22, 23, 24, 25, 26, 27, 28, 29, 30, 31, 32, 33, 34, 35, 36, 37, 38, 39, or 40 base pairs) and at least one base pair comprising a 5-methyluridine base paired with a 2,6-diaminopurine (“drtRNA”).

In a related embodiment, the drtRNA comprises at least two base pairs, each base pair comprising a 5-methyluridine base paired with a 2,6-diaminopurine.

In yet another embodiment, the drtRNA comprises at least three base pairs, each base pair comprising a 5-methyluridine base paired with a 2,6-diaminopurine.

In yet another embodiment, the drtRNA comprises at least four, five, or more base pairs (including 6, 7, 8, 9, 10, 11, 12, 13, 14, 15, 16, 17, 18, 19, 20 or more base pairs), each base pair comprising a 5-methyluridine base paired with a 2,6-diaminopurine.

In yet another embodiment, the drtRNA comprises at least four, five, or more base pairs (including 6, 7, 8, 9, 10, 11, 12, 13, 14, 15, 16, 17, 18, 19, 20 or more base pairs), each base pair comprising a 5-methyluridine base paired with a 2,6-diaminopurine, and at least one 5-methyluridine that is not base paired with a 2,6-diaminopurine (e.g., a 5-methyluridine base paired with an adenine or any other nucleoside capable of forming a base pair with a 5-methyluridine).

In yet another embodiment, the drtRNA comprises at least four, five, or more base pairs (including 6, 7, 8, 9, 10, 11, 12, 13, 14, 15, 16, 17, 18, 19, 20 or more base pairs), each base pair comprising a 5-methyluridine base paired with a 2,6-diaminopurine, and at least one 2,6-diaminopurine that is not base paired with a 5-methyluridine (e.g., a 2,6-diaminopurine base paired with an uracil or any other nucleoside capable of forming a base pair with a 2,6-diaminopurine).

In some embodiments herein, the drtRNA comprising at least one, two, three, four, five, or more base pairs (including 6, 7, 8, 9, 10, 11, 12, 13, 14, 15, 16, 17, 18, 19, 20 or more base pairs), each base pair comprising a 5-methyluridine base paired with a 2,6-diaminopurine and all the other nucleosides in the drtRNA comprising standard nucleosides (e.g., adenosine, guanosine, cytidine).

In yet another embodiment, the drtRNA comprises one or more nucleotides having the formula:

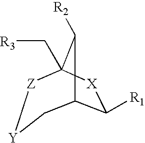

wherein, X is O or CH₂, Y is O, and Z is CH₂; R₁ is selected from the group consisting of adenine, cytosine, guanine, hypoxanthine, uracil, thymine, 2,6-diaminopurine, C-phenyl, C-naphthyl, inosine, azole carboxamide, 1-β-D-ribofuranosyl-4-nitroindole, 1-β-D-ribofuranosyl-5-nitroindole, 1-β-D-ribofuranosyl-6-nitroindole, or 1-β-D-ribofuranosyl-3-nitropyrrole, and a heterocycle wherein the heterocycle is selected from the group consisting of a substituted 1,3-diazine, unsubstituted 1,3-diazine, and an unsubstituted 7H imidazo[4,5]1,3 diazine; and R₂, R₃ are independently selected from a group consisting of H, OH, DMTO, TBDMSO, BnO, THPO, AcO, BzO, OP(NiPr₂)O(CH₂)₂CN, OPO₃ H, diphosphate, and triphosphate, wherein R₂ and R₃ together may be PhCHO₂, TIPDSO₂ or DTBSO₂.

In yet another embodiment, the drtRNA comprises one or more are locked nucleic acid (LNA) molecules.

In yet another embodiment, the drtRNA comprises one or more are universal-binding nucleotide. Non-limiting examples of universal-binding nucleotide include C-phenyl, C-naphthyl, inosine, azole carboxamide, 1-β-D-ribofuranosyl-4-nitroindole, 1-β-D-ribofuranosyl-5-nitroindole, 1-β-D-ribofuranosyl-6-nitroindole, or 1-β-D-ribofuranosyl-3-nitropyrrole. Within certain aspects, the present disclosure provides methods of using drtRNA that decreases expression of a target gene by RNAi, and compositions comprising one or more drtRNA, wherein at least one drtRNA comprises one or more universal-binding nucleotide(s) in the first, second or third position in the anti-codon of the antisense strand of the drtRNA duplex and wherein the drtRNA is capable of specifically binding to a target sequence, such as an RNA expressed by a cell. In cases wherein the sequence of the target RNA includes one or more single nucleotide substitutions, drtRNA comprising a universal-binding nucleotide retains its capacity to specifically bind a target RNA, thereby mediating gene silencing and, as a consequence, overcoming escape of the target from dsRNA-mediated gene silencing.

Non-limiting examples for the above compositions includes modifying the anti-codons for tyrosine (AUA) or phenylalanine (AAA or GAA), cysteine (ACA or GCA), histidine (AUG or GUG), asparagine (AUU or GUU), isoleucine (UAU) and aspartate (AUC or GUC) within the anti-codon of the antisense strand of the dsRNA molecule.

For example, within certain embodiments, the isoleucine anti-codon UAU, for which AUA is the cognate codon, may be modified such that the third-position uridine (U) nucleotide is substituted with the universal-binding nucleotide inosine (I) to create the anti-codon UAI. Inosine is an exemplary universal-binding nucleotide that can nucleotide-pair with an adenosine (A), uridine (U), and cytidine (C) nucleotide, but not guanosine (G). This modified anti-codon UAI increases the specific-binding capacity of the dsRNA molecule and thus permits the dsRNA to pair with mRNAs having any one of AUA, UUA, and CUA in the corresponding position of the coding strand thereby expanding the number of available RNA degradation targets to which the dsRNA may specifically bind.

Alternatively, the anti-codon AUA may also or alternatively be modified by substituting a universal-binding nucleotide in the third or second position of the anti-codon such that the anti-codon(s) represented by UAI (third position substitution) or UIU (second position substitution) to generate dsRNA that are capable of specifically binding to AUA, CUA and UUA and AAA, ACA and AUA.

In certain aspects, dsRNA disclosed herein can include between about 1 universal-binding nucleotide and about 10 universal-binding nucleotides. Within certain aspects, the presently disclosed dsRNA may comprise a sense strand that is homologous to a sequence of a target gene and an antisense strand that is complementary to the sense strand, with the proviso that at least one nucleotide of the antisense strand of the otherwise complementary dsRNA duplex is replaced by one or more universal-binding nucleotide.

It will be understood that, regardless of the position at which the one or more universal-binding nucleotide is substituted, the dsRNA molecule is capable of binding to a target gene and one or more variant(s) thereof thereby facilitating the degradation of the target gene or variant thereof via Dicer or a RISC complex. Thus, the dsRNA of the present disclosure are suitable for introduction into cells to mediate targeted post-transcriptional gene silencing of a TNF gene or variants thereof. When a dsRNA is inserted into a cell, the dsRNA duplex is then unwound, and the antisense strand anneals with mRNA to form a Dicer substrate or the antisense strand is loaded into an assembly of proteins to form the RNA-induced silencing complex (RISC).

In yet another embodiment, the drtRNA comprises one or more have a 2′-sugar substitution. Non-limiting examples of a 2′-sugar substitution include 2′-O-methyl, 2′-O-methoxyethyl, 2′-O-2-methoxyethyl, wherein the 2′-sugar substitution is a halogen, or wherein the 2′-sugar substitution is a 2′-fluoro, or wherein the 2′-sugar substitution is a 2′-O-allyl.

In yet another embodiment, the drtRNA comprises at least one nucleoside having a modified internucleoside linkage. Non-limiting example of a modified internucleoside linkage include a phosphorothioate, chiral phosphorothioate, phosphorodithioate, phosphotriester, aminoalkylphosphotriester, methyl phosphonate, alkyl phosphonate, 3′-alkylene phosphonate, 5′-alkylene phosphonate, chiral phosphonate, phosphonoacetate, thiophosphonoacetate, phosphinate, phosphoramidate, 3′-amino phosphoramidate, aminoalkylphosphoramidate, thionophosphoramidate, thionoalkylphosphonate, thionoalkylphosphotriester, selenophosphate, or boranophosphate linkage.

In yet another embodiment, the drtRNA has blunt ends.

In yet another embodiment, the drtRNA has one 3′ overhang of 1 to 5 (or 1, 2, 3, 4, 5) nucleotides.

In yet another embodiment, the drtRNA has two 3′ overhangs, each 3′ overhang having 1 to 5 (or 1, 2, 3, 4, 5) nucleotides.

In yet another embodiment, the drtRNA has an overhang of more than five nucleotides.

In yet another embodiment, the drtRNA comprises at least two double-stranded regions, each double-stranded region independently having about 10 to about 40 base pairs (or about 10, 11, 12, 13, 14, 15, 16, 17, 18, 29, 20, 21, 22, 23, 24, 25, 26, 27, 28, 29, 30, 31, 32, 33, 34, 35, 36, 37, 38, 39, or 40 base pairs), and at least one, two, three, four, five, or more base pairs (including 6, 7, 8, 9, 10, 11, 12, 13, 14, 15, 16, 17, 18, 19, 20 or more base pairs), each base pair comprising a 5-methyluridine base paired with a 2,6-diaminopurine, and where the double-stranded regions are spaced apart by up to 10 nucleotides (e.g., 1, 2, 3, 4, 5, 6, 7, 8, 9 or 10 nucleotides). In a related embodiment, the instant disclosure provides a dsRNA comprising at least two double-stranded regions, each double-stranded region independently having about 10 to about 40 base pairs (or about 10, 11, 12, 13, 14, 15, 16, 17, 18, 29, 20, 21, 22, 23, 24, 25, 26, 27, 28, 29, 30, 31, 32, 33, 34, 35, 36, 37, 38, 39, or 40 base pairs), and at least one, two, three, four, five, or more base pairs (including 6, 7, 8, 9, 10, 11, 12, 13, 14, 15, 16, 17, 18, 19, 20 or more base pairs), each base pair comprising a 5-methyluridine base paired with a 2,6-diaminopurine, and where the double-stranded regions are spaced apart by a nick.

In yet another embodiment, the drtRNA comprising any one or more embodiments disclosed herein.

In yet another embodiment, the drtRNA comprising at least one, two, three, four, five, or more base pairs (including 6, 7, 8, 9, 10, 11, 12, 13, 14, 15, 16, 17, 18, 19, 20 or more base pairs), each base pair comprising a 5-methyluridine base paired with a 2,6-diaminopurine, and wherein the 5-methyluridine of each base pair is in the guide strand (antisense strand) or wherein the 5-methyluridine of each base pair is in the passenger strand (sense strand).

In any one embodiment of the disclosure, a base pair comprising a 5-methyluridine base paired with a 2,6-diaminopurine in a double-stranded region of the RNA may be as follows:

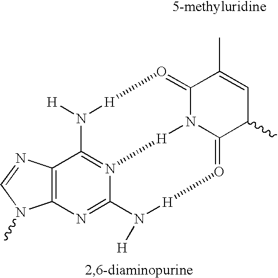

In this schematic, the 5-methyluridne:2,6-diaminopurine base pair has three hydrogen bonds (a hashed line represents a hydrogen bond). The base pair may have 1, 2 or 3 hydrogen bonds, preferably the base pair has 1 hydrogen bond, more preferably two hydrogen bonds and most preferably three hydrogen bonds.

In anyone embodiment of the disclosure, the double-stranded region of an RNA comprises at least one non-standard base pair comprising:

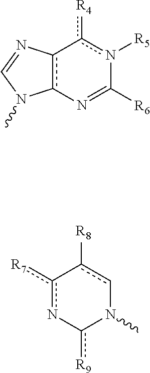

In this schematic, R₄, R₅, R₆, R₇, and R₈ are independently any one or more organic group consisting of one to twenty (1, 2, 3, 4, 5, 6, 7, 8, 9, 10, 11, 12, 13, 14, 15, 16, 17, 18, 19, or 20) atoms selected from carbon, oxygen, nitrogen, sulfur, hydrogen, selenium, silicon, halogen, chlorine, fluorine, and bromine. A dashed line indicates an optional bond that is either present or absent within the structures above. In this schematic, hydrogen bonds are not indicated in the above structures; however, such hydrogen bonds would form between the hydrogen bond donor and hydrogen bond acceptor groups of R₄ and R₇, between the hydrogen bond donor group of R₅ and the nitrogen (a hydrogen bond acceptor) in the third position of the pyrimidine structure above, and between the hydrogen bond donor and hydrogen bond acceptor group of R₆ and R₉. In an embodiment of this disclosure, R₄ has a hydrogen bond donor group and R₇ has a hydrogen bond acceptor group, R₅ has a hydrogen bond donor group, and R₆ has a hydrogen bond donor group and R₉ has an hydrogen bond acceptor group. In another embodiment, R₄ has an hydrogen bond acceptor group and R₇ has a hydrogen bond donor group, R₅ has a hydrogen bond donor group, and R₆ has a hydrogen bond donor group and R₉ has an hydrogen bond acceptor group. In another embodiment, R₄ has an hydrogen bond acceptor group and R₇ has a hydrogen bond donor group, R₅ has a hydrogen bond donor group, and R₆ has an hydrogen bond acceptor group and R₉ has a hydrogen bond donor group.

In anyone embodiment of the disclosure, the double-stranded region of an RNA comprises at least one non-standard base pair comprising:

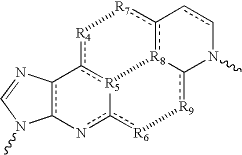

In this schematic, R₄, R₅, R₆, R₇, R₈, and R₉ are independently any one or more organic group consisting of one to twenty (1, 2, 3, 4, 5, 6, 7, 8, 9, 10, 11, 12, 13, 14, 15, 16, 17, 18, 19, or 20) atoms selected from carbon, oxygen, nitrogen, sulfur, hydrogen, selenium, silicon, halogen, chlorine, fluorine, and bromine. A dashed line indicates an optional bond that is either present or absent within the structures above. A hashed line between the substituent groups of the two structures above indicates the presence of a hydrogen bond (three hydrogen bonds are shown in this schematic). In another embodiment, R₄ has a donor group and R₇ has an acceptor group. In another embodiment, R₄ has an acceptor group and R₇ has an acceptor group. In another embodiment, R₅ has a donor group and R₈ has an acceptor group. In another embodiment, R₅ has an acceptor group and R₈ has an acceptor group. In another embodiment, R₆ has a donor group and R₉ has an acceptor group. In another embodiment, R₆ has an acceptor group and R₉ has an acceptor group.

In one embodiment, the instant disclosure provides a method for activating RNAi against a specific target gene by administering a dsRNA molecule to a cell expressing the target gene in an amount sufficient to reduce expression of the target gene by RNAi with a minimal off-target effect, wherein the dsRNA comprises a double-stranded region having about 10 to about 40 base pairs (or about 10, 11, 12, 13, 14, 15, 16, 17, 18, 29, 20, 21, 22, 23, 24, 25, 26, 27, 28, 29, 30, 31, 32, 33, 34, 35, 36, 37, 38, 39, or 40 base pairs) and at least one, two, three, four, five, or more base pairs (including 6, 7, 8, 9, 10, 11, 12, 13, 14, 15, 16, 17, 18, 19, 20 or more base pairs), each base pair comprising a 5-methyluridine base paired with a 2,6-diaminopurine, that decreases expression of a target gene by RNAi.

In one embodiment, the instant disclosure provides a method for activating RNAi against a specific target gene by administering a drtRNA molecule to a cell expressing the target gene in an amount sufficient to reduce expression of the target gene by RNAi with a minimal off-target effect.

In another embodiment, the disclosure provides a method for activating target gene-specific RNA interference (RNAi), comprising administering a drtRNA that decreases expression of a target gene by RNAi to a cell expressing the target gene.

In another embodiment, the disclosure provides a method of preparing a drtRNA that decreases expression of a target gene by RNAi, comprising (a) synthesizing a first strand and a second strand, wherein each strand has a length of from 10 to 60 nucleotides (or 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides), and wherein the first strand contains at least one 2,6-diaminopurine and the second strand contains at least one 5-methyluridine and (b) combining the first strand and the second strand to form a double-stranded RNA, wherein the double-stranded RNA contains a double-stranded region having from 10 to 40 base pairs (or about 10, 11, 12, 13, 14, 15, 16, 17, 18, 29, 20, 21, 22, 23, 24, 25, 26, 27, 28, 29, 30, 31, 32, 33, 34, 35, 36, 37, 38, 39, or 40 base pairs), and wherein the double-stranded region contains at least one, two, three, four, five, or more base pairs (including 6, 7, 8, 9, 10, 11, 12, 13, 14, 15, 16, 17, 18, 19, 20 or more base pairs), each base pair comprising a 5-methyluridine base paired with a 2,6-diaminopurine.

In any of the embodiments herein, the instant disclosure provides a method for activating RNAi against a specific target gene by administering a dsRNA having at least one base pair comprising a 5-methyluridine base paired with a 2,6-diaminopurine and at least one 5-methyluridine that is not base paired with a 2,6-diaminopurine (e.g., a 5-methyluridine base paired with an adenine or nucleoside capable of base pairing with a 5-methyluridine). In related embodiments, the double-stranded region of the dsRNA can comprise a guanosine nucleoside base paired with a cytosine nucleoside, a thymidine nucleoside base paired with a uridine nucleoside, and at least one 5-methyluridine base paired with a 2,6-diaminopurine. In other related embodiments, the double-stranded region of the dsRNA can comprise at least one adenosine nucleoside base paired with a uridine nucleoside, and at least one 5-methyluridine base paired with a 2,6-diaminopurine. In still further related embodiments, the double-stranded region of the dsRNA can comprise at least one guanosine or isoguanine (2-hydroxyladenine) nucleoside base paired with a 5-methyluridine nucleoside.

In any of the embodiments herein, the instant disclosure provides a dsRNA having at least one non-standard base pair that provide a dsRNA having improved RNAi capacity (e.g., minimized off-target effect, improved stability, improved potency, or minimized interferon response). Non-limiting examples of non-standard base pairs include a uracil base paired with a 2,6-diaminopurine; an isocytosine based paired with an isoguanine; an isocytosine base paired with a guanine; an isoguanine base paired with a cytosine; a diamino pyrimidine (5-methyl-4,5-dihydropyrimidine-2,4-diamine) base paired with a xanthosine; a 2,6-diaminopurine base paired with a xanthosine; a 2-amino-pyrazine-6-one (6-amino-3,5-dimethylpyrazin-2(1H)-one) base paired with a 5-aza-7-deaza-isoguanine (4-aminoimidazo[1,2-a][1,3,5]triazin-2(8H)-one); a 6-amino-pyrazine-2-one (6-amino-3,5-dimethylpyrazin-2(1H)-one) base paired with a 5-aza-7-deaza-guanine (7-aminoimidazo[1,2-c]pyrimidin-5(1H)-one); an inosine base paired with a 5-methylurdine; an inosine base paired with a 2,6-diaminopurine.

In any of the embodiments herein, the instant disclosure provides an RNA comprising a double-stranded region having at least one base pair having two hydrogen bonds substituted with a base pair having three hydrogen bonds, wherein the substituted base pair having three hydrogen bonds provide the RNA with improved RNAi capacity (e.g., minimized off-target effect, improved stability, improved potency, or minimized interferon response).

In any of the embodiments herein, the instant disclosure provides an RNA comprising a first strand that is complementary to a target nucleic acid (e.g., target gene mRNA), a second strand that is complementary to the first strand, wherein the first and second strands of the RNA form a double-stranded region of about 10 to about 40 base pairs (or about 10, 11, 12, 13, 14, 15, 16, 17, 18, 29, 20, 21, 22, 23, 24, 25, 26, 27, 28, 29, 30, 31, 32, 33, 34, 35, 36, 37, 38, 39, or 40 base pairs), and wherein the double-stranded region contains at least one, two, three, four, five, or more base pairs (including 6, 7, 8, 9, 10, 11, 12, 13, 14, 15, 16, 17, 18, 19, 20 or more base pairs), each base pair comprising a 5-methyluridine base paired with a 2,6-diaminopurine. In a related embodiment, the first strand may be a guide strand (antisense strand) and the second strand may be a passenger strand (sense strand). In yet another embodiment, the second strand may be a guide strand (antisense strand) and the first strand may be a passenger strand (sense strand). In a related embodiment, the 5-methyluridine of a 5-methyluridine:2,6-diaminopurine base pair is present only in the first strand. In a related embodiment, the 5-methyluridine of a 5-methyluridine:2,6-diaminopurine base pair is present only in the second strand.

In particular embodiments, there are provided methods of treating or preventing diseases, disorders, or conditions related to gene expression, including those related, or responsive, to the level of a target nucleic acid molecule (e.g., mRNA) in a cell or tissue, by administering a dsRNA molecule of this disclosure, alone or in combination with an adjunctive therapy, in an amount sufficient to activate target gene-specific RNAi. In one embodiment, there is provided a method of treating or preventing a disease or disorder by administering a dsRNA molecule that is capable of target gene-specific RNAi, which dsRNA has at least one base pair comprising a 5-methyluridine base paired with a 2,6-diaminopurine as described herein and has a reduced or minimal off-target effect.

As used herein, reference to a target mRNA or target RNA sequence or sense strand means a human target nucleic acid sequence as set forth in any one particular accession number of Table A, as well as variants, isoforms and, homologs having at least 70% or more identity (i.e., 70%, 75%, 80%, 85%, 90%, 95% or 100%) with the human target nucleic acid sequence.

The content of Table A has been submitted to the U.S. Patent and Trademark Office as a separate text file, named “Table_B_Human-RefSeq_Accession-Numbers.txt” (please see attached “Table B. Human RefSeq Accession Numbers), and is incorporated herein by reference in its entirety.

The percent identity between two or more sequences is a function of the number of identical positions shared by the sequences (i.e., % identity=number of identical positions/total number of positions×100), taking into account the number of gaps and the length of each gap that need to be introduced to optimize alignment of two or more sequences. The comparison of sequences and determination of percent identity between two or more sequences can be accomplished using a mathematical algorithm, such as BLAST and Gapped BLAST programs at their default parameters (e.g., BLASTN, see www.ncbi.nlm.nih.gov/BLAST; see also, Altschul et al., J. Mol. Biol. 215:403-410, 1990).

Another aspect of this disclosure is the use of a dsRNA of this disclosure in the manufacture of a medicament for treating a disease in a subject by inhibiting expression of a target gene in the subject, such as a human. Another aspect of this disclosure includes a pharmaceutical formulation for treating a disease in a subject comprising dsRNA, wherein the dsRNA is capable of altering expression of a target gene in cells of the subject (e.g., human or non-human mammal). In certain embodiments, the disease is a systemic disease. In other embodiments, the disease is an inflammatory or autoimmune disease, such as rheumatoid arthritis. In an embodiment of this disclosure, the formulation can be administered to the circulation of a mammal, such as by intravenous administration. In another embodiment, the dsRNA is delivered to blood leucocytes, such as monocytes. In another embodiment, administration of a dsRNA formulation of this disclosure decreases the levels of a target gene in the circulation of a mammal. In certain embodiments, the mammal or cell is a human or human cell, respectively. In further embodiments, a target sequence is a human target nucleic acid sequence as set forth in any one of the accession numbers of Table A.

In an exemplary embodiment, a dsRNA molecule comprising a first strand that is complementary to the target gene mRNA, a second strand that is complementary to the first strand, wherein the first and second strands of the dsRNA form a double-stranded region of about 10 to about 40 base pairs (or about 10, 11, 12, 13, 14, 15, 16, 17, 18, 29, 20, 21, 22, 23, 24, 25, 26, 27, 28, 29, 30, 31, 32, 33, 34, 35, 36, 37, 38, 39, or 40 base pairs), and wherein the double-stranded region contains at least one, two, three, four, five, or more base pairs (including 6, 7, 8, 9, 10, 11, 12, 13, 14, 15, 16, 17, 18, 19, 20 or more base pairs), each base pair comprising a 5-methyluridine base paired with a 2,6-diaminopurine, and wherein the dsRNA decreases expression of a target gene by RNAi and has a minimal off-target effect is useful as a therapeutic tool to regulate expression of a target gene to treat or prevent symptoms of rheumatoid arthritis (RA). In other embodiments, the dsRNA molecule for use in treating or preventing RA can optionally be included in a pharmaceutically acceptable formulation comprising a delivery vehicle, carrier, or diluent, or the dsRNA molecule can be combined with a disease-modifying antirheumatic drug described herein or otherwise known in the art (e.g., nonsteroidal anti-inflammatory drug, analgesic, methotrexate, hydroxychloroquine, sulfasalazine, leflunomide, etanercept, infliximab, prednisone).

An exemplary target gene for treating RA or other inflammatory or autoimmune diseases is a tumor necrosis factor gene (TNF, formerly known as TNF-α). A TNF gene encodes for a multifunctional proinflammatory cytokine secreted predominantly by monocytes and macrophages that has effects on many processes, including cell survival, inflammation, and immunity. The complete human TNF mRNA sequence of has Genbank accession number NM_{—}000594.2 and exemplary dsRNA molecules for use in the methods of this disclosure can be found, for example, in Table 1.

In another exemplary embodiment, a drtRNA decreases expression of a target gene by RNAi and has a minimal off-target effect is useful as a therapeutic tool to regulate expression of a target gene to treat or prevent metabolic syndrome, including high blood pressure, obesity, cardiovascular disease, diabetes, or any combination thereof. In related embodiments, the drtRNA may be for use in treating or preventing a metabolic disease can optionally be included in a pharmaceutically acceptable formulation comprising a delivery vehicle, carrier, or diluent, or the drtRNA can be combined with a disease-modifying antirheumatic drug described herein or otherwise known in the art (e.g., nonsteroidal anti-inflammatory drug, analgesic, methotrexate, hydroxychloroquine, sulfasalazine, leflunomide, etanercept, infliximab, prednisone).

As can be readily determined from this disclosure, useful dsRNAs having multiple substitutions or modifications will retain their RNAi activity. The dsRNA molecules of the instant disclosure thus provide useful reagents and methods for a variety of therapeutic, diagnostic, target validation, genomic discovery, genetic engineering, and pharmacogenomic applications.

Exemplary dsRNA Molecules for Silencing a Target Gene via RNAi

The following exemplary dsRNAs of the present disclosure are shown with both the antisense (first) and sense (second) strands in the 5′ to 3′ orientation. For the following exemplary dsRNAs, the (dTdT) nucleotides of each strand represent overhangs that do not necessarily pair with any nucleotides in the opposing strand of the same dsRNA. In certain embodiments, a dsRNA may contain one base pair with a 5-methyluridine base paired with a 2,6-diaminopurine, wherein the 5-methyluridine of a 5-methyluridine:2,6-diaminopurine base pair is in the sense strand or the antisense strand. In certain embodiments, a dsRNA may contain more than one base pair with a 5-methyluridine base paired with a 2,6-diaminopurine, wherein the 5-methyluridine of a 5-methyluridine:2,6-diaminopurine base pair is in the sense strand or the antisense strand. In certain embodiments, a dsRNA may contain more than one base pair with a 5-methyluridine base paired with a 2,6-diaminopurine, wherein the sense strand and the antisense strand of the dsRNA have a 5-methyluridine of a 5-methyluridine:2,6-diaminopurine base pair. In any embodiment disclosed herein, the 5-methyluridine:2,6-diaminopurine in the double-stranded region of a dsRNA improves the capacity of the dsRNA to mediate RNAi (e.g., minimized off-target effect, improved stability, improved potency, or minimized interferon response). In further embodiments, the dsRNA may be further modified, for example, at the 2′-O position of the ribose (e.g., 2′-O-alkyl such as 2′-O-methyl or 2′-O-methoxyethyl). A 5-methyluridine within a dsRNA is indicated by a “‘t”; a 2,6-diaminopurine within a dsRNA is indicated by a “A^{2/6}”; a nucleotide having a 2′-O-methyl modification is indicated with an underline, N (N being any nucleotide as described herein); and a “p” at the 5′-end indicates that it is phosphorylated (although not shown on all strands, any of the exemplary sequences can have a phosphorylated 5′-end).

Generally, any RNA having a double-stranded region or having the potential for form a double-stranded region (e.g., forms a double-stranded region from two or more strands, or from a single strand that forms a stem-loop or hairpin structure) comprising at least one base pair comprising an adenine base paired with a uracil may be selected as a dsRNA to have a base pair comprising a 5-methyluridine based paired with a 2,6-diaminopurine. Once a adenine:uracil base pair is identified within the double-stranded region of a dsRNA, the identified adenine or adenines within the RNA may be replaced with 2,6-diaminopurine and the complementary uracil or uracils within the RNA may be replaced with 5-methyluridine such that the adenine:uracil base pair or base pairs are replaced with the 5-methyluridine:2,6-diaminopurine base pair or base pairs.

Generally, any RNA having a double-stranded region or having the potential for form a double-stranded region (e.g., forms a double-stranded region from two or more strands, or from a single strand that forms a stem-loop or hairpin structure) may have at least one base pair added to the double-stranded region comprising a 5-methyluridine based paired with a 2,6-diaminopurine by addition of a 5-methylurine and a 2,6-diaminopurine to the RNA such that upon formation of a double-stranded region in the RNA the 5-methyluridine forms a base pair with the 2,6-diaminopurine.

The RNAs below have one or more base pairs comprising a 5-methyluridine based paired with a 2,6-diaminopurine and serve as non-limiting examples of how the same dsRNA may have one or more base pairs comprising a 5-methyluridine based paired with a 2,6-diaminopurine.

An exemplary dsRNA duplex of the present disclosure that would target the RNA of Hepatitis B virus and target a subsequence of the HBV RNA would be:

Antisense:

(SEQ ID NO: 192)

G A^{2/6} t G A^{2/6} G G C A^{2/6} t A^{2/6} G C A^{2/6} G C
A^{2/6} G G (dT dT)
Sense:

(SEQ ID NO: 193)

C C t G C t G C t A^{2/6} t G C C t C A^{2/6} t C
(dT dT)
or
Antisense:

(SEQ ID NO: 194)

G A^{2/6} U G A^{2/6} G G C A^{2/6} U A^{2/6} G C A^{2/6} G C
A^{2/6} G G (dT dT)
Sense:

(SEQ ID NO: 195)

C C t G C t G C t A t G C C t C A t C (dT dT)
or
Antisense:

(SEQ ID NO: 196)

G A t G A G G C A t A G C A G C A G G (dT dT)
Sense:

(SEQ ID NO: 197)

C C U G C U G C U A^{2/6} U G C C U C A^{2/6} U C
(dT dT)
or
Antisense:

(SEQ ID NO: 198)

G A^{2/6} t G A^{2/6} G G C A U A G C A G C A G G
(dT dT)
Sense:

(SEQ ID NO: 199)

C C U G C U G C U A U G C C t C A^{2/6} t C (dT dT)
or
Antisense:

(SEQ ID NO: 200)

G A U G A G G C A U A G C A^{2/6} G C A^{2/6} G G
(dT dT)
Sense:

(SEQ ID NO: 201)

C C t G C t G C U A U G C C U C A U C (dT dT)
or

Generally, with the dsRNA shown below, any one or more adenines may be replaced with a 2,6-diaminopurine and the complementary uracil may be replaced with a 5-methyluridine.

Antisense:

(SEQ ID NO: 202)

G A U G A G G C A U A G C A G C A G G (dT dT)
Sense:

(SEQ ID NO: 203)

C C U G C U G C U A U G C C U C A U C (dT dT)

For further representative dsRNA sequences that target HBV, see United States Patent Application Publication No. 2003/0206887, published Nov. 6, 2003.

An exemplary dsRNA duplex of the present disclosure that would target RNA of the human immunodeficiency virus (HIV) is:

Antisense:

(SEQ ID NO: 204)

t t t G C t G G t C C t t t C C A^{2/6} A^{2/6} A^{2/6}
(dT dT)
Sense:

(SEQ ID NO: 205)

t t t G G A^{2/6} A^{2/6} A^{2/6} G G A^{2/6} C C A^{2/6} G C
A^{2/6} A^{2/6} A^{2/6} (dT dT)

Generally, with the dsRNA shown below, any one or more adenine may be replaced with a 2,6-diaminopurine and the complementary uracil may be replaced with a 5-methyluridine.

Antisense:

(SEQ ID NO: 206)

U U U G C U G G U C C U U U C C A A A (dT dT)
Sense:

(SEQ ID NO: 207)

U U U G G A A A G G A C C A G C A A A (dT dT)

For further representative dsRNA sequences that target HIV, see United States Patent Application Publication No. 2003/0175950, published Sep. 18, 2003.

Exemplary dsRNA duplexes of the present disclosure that would target mRNA of TNF-receptor 1A include those found in PCT Application Publication No. WO 03/070897, such as:

Antisense:

(SEQ ID NO: 208)

C t G G G G C t t C C C G G G A^{2/6} C t C (dT dT)
Sense:

(SEQ ID NO: 209)

G A^{2/6} G t C C C G G G A^{2/6} A^{2/6} G C C C C A^{2/6} G
(dT dT)
Antisense:

(SEQ ID NO: 210)

t G t A^{2/6} C A^{2/6} A^{2/6} G t A^{2/6} G G t t C C t t t
(dT dT)
Sense:

(SEQ ID NO: 211)

A^{2/6} A^{2/6} A^{2/6} G G A^{2/6} A^{2/6} C C t A^{2/6} C t t G t
A^{2/6} C A^{2/6} (dT dT)

Exemplary dsRNA duplexes of the present disclosure that would target mRNA of human tumor necrosis factor (TNF or hTNF) include:

Antisense:

(SEQ ID NO: 212)

C t GGCA^{2/6}GC t t G t CA^{2/6}GGG t G (dTdT)
Sense:

(SEQ ID NO: 213)

CA^{2/6}CCC t GA^{2/6}CA^{2/6}A^{2/6}GC t GCCA^{2/6}G (dTdT)
Antisense:

(SEQ ID NO: 214)

CCGA^{2/6} t CA^{2/6}C t CCA^{2/6}A^{2/6}A^{2/6}G t GCA^{2/6} (dTdT)
Sense:

(SEQ ID NO: 215)

t GCA^{2/6}C t t t GGA^{2/6}G t GA^{2/6} t CGG (dTdT)
Antisense:

(SEQ ID NO: 216)

pAAGGA^{2/6}GA^{2/6}A^{2/6}GA^{2/6}GGC t GA^{2/6}GGA (dTdT)
Sense:

(SEQ ID NO: 217)

t CC t CA^{2/6}GCC t C t t C t CCUU (dTdT)
Antisense:

(SEQ ID NO: 218)

P t AA^{2/6}GC t t GGG t t CCGACCC (dTdA)
Sense:

(SEQ ID NO: 219)

GGG t CGGA^{2/6} A^{2/6}CCCA^{2/6} A^{2/6} GC t UA^{2/6} (dTdT)

Generally, with the dsRNA shown above, any one or more adenine may be replaced with a 2,6-diaminopurine and the complementary uracil may be replaced with a 5-methyluridine.

These sequences would be useful as dsRNA in treating TNF-associated diseases in a human, such as rheumatoid arthritis, as well as other inflammatory or autoimmune diseases or disorders.

Further exemplary dsRNA of the present disclosure targeted against the TNF mRNA are provided in Table 1—shown is the sense strand only. (See, also, PCT Patent Application Publication No. WO 03/070897.) It should be understood that at least one of the uridines in the sequences of Table 1 or the complementary strand of these sequences is substituted with a 5-methyluridine and the complementary adenine of the substituted uridines in the sequences of Table 1 is substituted with a 2,6-diaminopurine. For example, dsRNA molecules are provided that decrease expression of a tumor necrosis factor (TNF) gene by RNA interference, comprising a first (antisense) strand that is complementary to TNF mRNA set forth in SEQ ID NO: 182 and a second (sense) strand that is complementary to the first strand, wherein the first and second strands form a double-stranded region of about 10 to about 40 base pairs, and the double-stranded region contains at least one, two, three, four, five, or more base pairs (including 6, 7, 8, 9, 10, 11, 12, 13, 14, 15, 16, 17, 18, 19, 20 or more base pairs), each base pair comprising a 5-methyluridine base paired with a 2,6-diaminopurine.

As provided herein, these would be useful in treating TNF-associated diseases or disorders, such as inflammatory disorders, autoimmune disorders, or rheumatoid arthritis. In certain embodiments, the dsRNA molecules are those provided in Table 1.

TABLE 1
Sense Strand of dsRNA Useful for Targeting
TNF by RNAi

| | | |
|---|---|---|
| TNF | | SEQ ID |
| Position | dsRNA Sequence | NO. |

BASED ON TNF (NM_000594.2)

| | | |
|---|---|---|
| 3 | CCCUCAGCAAGGACAGCAG | 1 |
| 21 | GAGGACCAGCUAAGAGGGA | 2 |
| 39 | AGAGAAGCAACUACAGACC | 3 |
| 57 | CCCCCCUGAAAACAACCCU | 4 |
| 75 | UCAGACGCCACAUCCCCUG | 5 |
| 93 | GACAAGCUGCCAGGCAGGU | 6 |
| 111 | UUCUCUUCCUCUCACAUAC | 7 |
| 129 | CUGACCCACGGCUCCACCC | 8 |
| 147 | CUCUCUCCCCUGGAAAGGA | 9 |
| 165 | ACACCAUGAGCACUGAAAG | 10 |
| 183 | GCAUGAUCCGGGACGUGGA | 11 |
| 201 | AGCUGGCCGAGGAGGCGCU | 12 |
| 219 | UCCCCAAGAAGACAGGGGG | 13 |
| 237 | GGCCCCAGGGCUCCAGGCG | 14 |
| 255 | GGUGCUUGUUCCUCAGCCU | 15 |
| 273 | UCUUCUCCUUCCUGAUCGU | 16 |
| 291 | UGGCAGGCGCCACCACGCU | 17 |
| 309 | UCUUCUGCCUGCUGCACUU | 18 |
| 327 | UUGGAGUGAUCGGCCCCCA | 19 |
| 345 | AGAGGGAAGAGUUCCCCAG | 20 |
| 363 | GGGACCUCUCUCUAAUCAG | 21 |
| 381 | GCCCUCUGGCCCAGGCAGU | 22 |
| 399 | UCAGAUCAUCUUCUCGAAC | 23 |
| 417 | CCCCGAGUGACAAGCCUGU | 24 |
| 435 | UAGCCCAUGUUGUAGCAAA | 25 |
| 453 | ACCCUCAAGCUGAGGGGCA | 26 |
| 471 | AGCUCCAGUGGCUGAACCG | 27 |
| 489 | GCCGGGCCAAUGCCCUCCU | 28 |
| 507 | UGGCCAAUGGCGUGGAGCU | 29 |
| 525 | UGAGAGAUAACCAGCUGGU | 30 |
| 543 | UGGUGCCAUCAGAGGGCCU | 31 |
| 561 | UGUACCUCAUCUACUCCCA | 32 |
| 579 | AGGUCCUCUUCAAGGGCCA | 33 |
| 597 | AAGGCUGCCCCUCCACCCA | 34 |
| 615 | AUGUGCUCCUCACCCACAC | 35 |
| 633 | CCAUCAGCCGCAUCGCCGU | 36 |
| 651 | UCUCCUACCAGACCAAGGU | 37 |
| 669 | UCAACCUCCUCUCUGCCAU | 38 |
| 687 | UCAAGAGCCCCUGCCAGAG | 39 |
| 705 | GGGAGACCCCAGAGGGGGC | 40 |
| 723 | CUGAGGCCAAGCCCUGGUA | 41 |
| 741 | AUGAGCCCAUCUAUCUGGG | 42 |
| 759 | GAGGGGUCUUCCAGCUGGA | 43 |
| 777 | AGAAGGGUGACCGACUCAG | 44 |
| 795 | GCGCUGAGAUCAAUCGGCC | 45 |
| 813 | CCGACUAUCUCGACUUUGC | 46 |
| 831 | CCGAGUCUGGGCAGGUCUA | 47 |
| 849 | ACUUUGGGAUCAUUGCCCU | 48 |
| 867 | UGUGAGGAGGACGAACAUC | 49 |
| 885 | CCAACCUUCCCAAACGCCU | 50 |
| 903 | UCCCCUGCCCCAAUCCCUU | 51 |
| 921 | UUAUUACCCCCUCCUUCAG | 52 |
| 939 | GACACCCUCAACCUCUUCU | 53 |
| 957 | UGGCUCAAAAAGAGAAUUG | 54 |
| 975 | GGGGGCUUAGGGUCGGAAC | 55 |
| 993 | CCCAAGCUUAGAACUUUAA | 56 |
| 1011 | AGCAACAAGACCACCACUU | 57 |
| 1029 | UCGAAACCUGGGAUUCAGG | 58 |
| 1047 | GAAUGUGUGGCCUGCACAG | 59 |
| 1065 | GUGAAGUGCUGGCAACCAC | 60 |
| 1083 | CUAAGAAUUCAAACUGGGG | 61 |
| 1101 | GCCUCCAGAACUCACUGGG | 62 |
| 1119 | GGCCUACAGCUUUGAUCCC | 63 |
| 1137 | CUGACAUCUGGAAUCUGGA | 64 |
| 1155 | AGACCAGGGAGCCUUUGGU | 65 |
| 1173 | UUCUGGCCAGAAUGCUGCA | 66 |
| 1191 | AGGACUUGAGAAGACCUCA | 67 |
| 1209 | ACCUAGAAAUUGACACAAG | 68 |
| 1227 | GUGGACCUUAGGCCUUCCU | 69 |
| 1245 | UCUCUCCAGAUGUUUCCAG | 70 |
| 1263 | GACUUCCUUGAGACACGGA | 71 |
| 1281 | AGCCCAGCCCUCCCCAUGG | 72 |
| 1299 | GAGCCAGCUCCCUCUAUUU | 73 |
| 1317 | UAUGUUUGCACUUGUGAUU | 74 |
| 1335 | UAUUUAUUAUUUAUUUAUU | 75 |
| 1353 | UAUUUAUUUAUUUACAGAU | 76 |
| 1371 | UGAAUGUAUUUAUUUGGGA | 77 |
| 1389 | AGACCGGGGUAUCCUGGGG | 78 |
| 1407 | GGACCCAAUGUAGGAGCUG | 79 |
| 1425 | GCCUUGGCUCAGACAUGUU | 80 |
| 1443 | UUUCCGUGAAAACGGAGCU | 81 |
| 1461 | UGAACAAUAGGCUGUUCCC | 82 |
| 1479 | CAUGUAGCCCCCUGGCCUC | 83 |
| 1497 | CUGUGCCUUCUUUUGAUUA | 84 |
| 1515 | AUGUUUUUUAAAAUAUUUA | 85 |
| 1533 | AUCUGAUUAAGUUGUCUAA | 86 |
| 1551 | AACAAUGCUGAUUUGGUGA | 87 |
| 1569 | ACCAACUGUCACUCAUUGC | 88 |
| 1587 | CUGAGCCUCUGCUCCCCAG | 89 |
| 1605 | GGGGAGUUGUGUCUGUAAU | 90 |
| 1623 | UCGCCCUACUAUUCAGUGG | 91 |
| 1641 | GCGAGAAAUAAAGUUUGCU | 92 |
| 1649 | UAAAGUUUGCUUAGAAAAG | 93 |

BASED ON TNF (NM_000594.1)

| 3 | CACCCUGACAAGCUGCCAG | 94 |
|---|---|---|
| 21 | GGCAGGUUCUCUUCCUCUC | 95 |
| 39 | CACAUACUGACCCACGGCU | 96 |
| 57 | UCCACCCUCUCUCCCCUGG | 97 |
| 75 | GAAAGGACACCAUGAGCAC | 98 |
| 93 | CUGAAAGCAUGAUCCGGGA | 99 |
| 111 | ACGUGGAGCUGGCCGAGGA | 100 |
| 129 | AGGCGCUCCCCAAGAAGAC | 101 |
| 147 | CAGGGGGGCCCCAGGGCUC | 102 |
| 165 | CCAGGCGGUGCUUGUUCCU | 103 |
| 183 | UCAGCCUCUUCUCCUUCCU | 104 |
| 201 | UGAUCGUGGCAGGCGCCAC | 105 |
| 219 | CCACGCUCUUCUGCCUGCU | 106 |
| 237 | UGCACUUUGGAGUGAUCGG | 107 |
| 255 | GCCCCCAGAGGGAAGAGUC | 108 |
| 273 | CCCCCAGGGACCUCUCUCU | 109 |
| 291 | UAAUCAGCCCUCUGGCCCA | 110 |
| 309 | AGGCAGUCAGAUCAUCUUC | 111 |
| 327 | CUCGAACCCCGAGUGACAA | 112 |
| 345 | AGCCUGUAGCCCAUGUUGU | 113 |
| 363 | UAGCAAACCCUCAAGCUGA | 114 |
| 381 | AGGGGCAGCUCCAGUGGCU | 115 |
| 399 | UGAACCGCCGGGCCAAUGC | 116 |
| 417 | CCCUCCUGGCCAAUGGCGU | 117 |
| 435 | UGGAGCUGAGAGAUAACCA | 118 |
| 453 | AGCUGGUGGUGCCAUCAGA | 119 |
| 471 | AGGGCCUGUACCUCAUCUA | 120 |
| 489 | ACUCCCAGGUCCUCUUCAA | 121 |
| 507 | AGGGCCAAGGCUGCCCCUC | 122 |
| 525 | CCACCCAUGUGCUCCUCAC | 123 |
| 543 | CCCACACCAUCAGCCGCAU | 124 |
| 561 | UCGCCGUCUCCUACCAGAC | 125 |
| 579 | CCAAGGUCAACCUCCUCUC | 126 |
| 597 | CUGCCAUCAAGAGCCCCUG | 127 |
| 615 | GCCAGAGGGAGACCCCAGA | 128 |
| 633 | AGGGGGCUGAGGCCAAGCC | 129 |
| 651 | CCUGGUAUGAGCCCAUCUA | 130 |
| 669 | AUCUGGGAGGGGUCUUCCA | 131 |
| 687 | AGCUGGAGAAGGGUGACCG | 132 |
| 705 | GACUCAGCGCUGAGAUCAA | 133 |
| 723 | AUCGGCCCGACUAUCUCGA | 134 |
| 741 | ACUUUGCCGAGUCUGGGCA | 135 |
| 759 | AGGUCUACUUUGGGAUCAU | 136 |
| 777 | UUGCCCUGUGAGGAGGACG | 137 |
| 795 | GAACAUCCAACCUUCCCAA | 138 |
| 813 | AACGCCUCCCCUGCCCCAA | 139 |
| 831 | AUCCCUUUAUUACCCCCUC | 140 |
| 849 | CCUUCAGACACCCUCAACC | 141 |
| 867 | CUCUUCUGGCUCAAAAAGA | 142 |
| 885 | AGAAUUGGGGGCUUAGGGU | 143 |
| 903 | UCGGAACCCAAGCUUAGAA | 144 |
| 921 | ACUUUAAGCAACAAGACCA | 145 |
| 939 | ACCACUUCGAAACCUGGGA | 146 |
| 957 | AUUCAGGAAUGUGUGGCCU | 147 |
| 975 | UGCACAGUGAAGUGCUGGC | 148 |
| 993 | CAACCACUAAGAAUUCAAA | 149 |
| 1011 | ACUGGGGCCUCCAGAACUC | 150 |
| 1029 | CACUGGGGCCUACAGCUUU | 151 |
| 1047 | UGAUCCCUGACAUCUGGAA | 152 |
| 1065 | AUCUGGAGACCAGGGAGCC | 153 |
| 1083 | CUUUGGUUCUGGCCAGAAU | 154 |
| 1101 | UGCUGCAGGACUUGAGAAG | 155 |
| 1119 | GACCUCACCUAGAAAUUGA | 156 |
| 1137 | ACACAAGUGGACCUUAGGC | 157 |
| 1155 | CCUUCCUCUCUCCAGAUGU | 158 |
| 1173 | UUUCCAGACUUCCUUGAGA | 159 |
| 1191 | ACACGGAGCCCAGCCCUCC | 160 |
| 1209 | CCCAUGGAGCCAGCUCCCU | 161 |
| 1227 | UCUAUUUAUGUUUGCACUU | 162 |
| 1245 | UGUGAUUAUUUAUUAUUUA | 163 |
| 1263 | AUUUAUUAUUUAUUUAUUU | 164 |
| 1281 | UACAGAUGAAUGUAUUUAU | 165 |
| 1299 | UUUGGGAGACCGGGGUAUC | 166 |
| 1317 | CCUGGGGGACCCAAUGUAG | 167 |
| 1335 | GGAGCUGCCUUGGCUCAGA | 168 |
| 1353 | ACAUGUUUUCCGUGAAAAC | 169 |
| 1371 | CGGAGGCUGAACAAUAGGC | 170 |
| 1389 | CUGUUCCCAUGUAGCCCCC | 171 |
| 1407 | CUGGCCUCUGUGCCUUCUU | 172 |
| 1425 | UUUGAUUAUGUUUUUUAAA | 173 |
| 1443 | AAUAUUAUCUGAUUAAGUU | 174 |
| 1461 | UGUCUAAACAAUGCUGAUU | 175 |
| 1479 | UUGGUGACCAACUGUCACU | 176 |
| 1497 | UCAUUGCUGAGGCCUCUGC | 177 |
| 1515 | CUCCCCAGGGAGUUGUGUC | 178 |
| 1533 | CUGUAAUCGGCCUACUAUU | 179 |
| 1551 | UCAGUGGCGAGAAAUAAAG | 180 |
| 1565 | UAAAGGUUGCUUAGGAAAG | 181 |
| Full Length | CUCCCUCAGCAAGGACAGCAGAGGACCA | 182 |
| TNF 1-1669 | GCUAAGAGGGAGAGAAGCAACUACAGA | |
| (NM_000549.2) | CCCCCCCUGAAAACAACCCUCAGACGCC | |
| | ACAUCCCCUGACAAGCUGCCAGGCAGGU | |
| | UCUCUUCCUCUCACAUACUGACCCACGG | |
| | CUCCACCCUCUCUCCCCUGGAAAGGACA | |
| | CCAUGAGCACUGAAAGCAUGAUCCGGGA | |
| | CGUGGAGCUGGCCGAGGAGGCGCUCCCC | |
| | AAGAAGACAGGGGGGCCCCAGGGCUCCA | |
| | GGCGGUGCUUGUUCCUCAGCCUCUUCUC | |
| | CUUCCUGAUCGUGGCAGGCGCCACCACG | |
| | CUCUUCUGCCUGCUGCACUUUGGAGUGA | |
| | UCGGCCCCCAGAGGGAAGAGUUCCCCAG | |
| | GGACCUCUCUCUAAUCAGCCCUCUGGCC | |
| | CAGGCAGUCAGAUCAUCUUCUCGAACCC | |
| | CGAGUGACAAGCCUGUAGCCCAUGUUGU | |
| | AGCAAACCCUCAAGCUGAGGGGCAGCUC | |
| | CAGUGGCUGAACCGCCGGGCCAAUGCCC | |
| | UCCUGGCCAAUGGCGUGGAGCUGAGAG | |
| | AUAACCAGCUGGUGGUGCCAUCAGAGG | |
| | GCCUGUACCUCAUCUACUCCCAGGUCCU | |
| | CUUCAAGGGCCAAGGCUGCCCCUCCACC | |
| | CAUGUGCUCCUCACCCACACCAUCAGCC | |
| | GCAUCGCCGUCUCCUACCAGACCAAGGU | |
| | CAACCUCCUCUCUGCCAUCAAGAGCCCC | |
| | UGCCAGAGGGAGACCCCAGAGGGGGCUG | |
| | AGGCCAAGCCCUGGUAUGAGCCCAUCUA | |
| | UCUGGGAGGGGUCUUCCAGCUGGAGAA | |
| | GGGUGACCGACUCAGCGCUGAGAUCAAU | |
| | CGGCCCGACUAUCUCGACUuUGCCGAGU | |
| | CUGGGCAGGUCUACUUUGGGAUCAUUG | |
| | CCCUGUGAGGAGGACGAACAUCCAACCU | |
| | UCCCAAACGCCUCCCCUGCCCCAAUCCC | |
| | UUUAUUACCCCCUCCUUCAGACACCCUC | |
| | AACCUCUUCUGGCUCAAAAAGAGAAUU | |
| | GGGGGCUUAGGGUCGGAACCCAAGCUU | |
| | AGAACUUUAAGCAACAAGACCACCACUU | |
| | CGAAACCUGGGAUUCAGGAAUGUGUGG | |
| | CCUGCACAGUGAAGUGCUGGCAACCACU | |
| | AAGAAUUCAAACUGGGGCCUCCAGAACU | |
| | CACUGGGGCCUACAGCUUUGAUCCCUGA | |
| | CAUCUGGAAUCUGGAGACCAGGGAGCCU | |
| | UUGGUUCUGGCCAGAAUGCUGCAGGAC | |
| | UUGAGAAGACCUCACCUAGAAAUUGAC | |
| | ACAAGUGGACCUUAGGCCUUCCUCUCUC | |
| | CAGAUGUUUCCAGACUUCCUUGAGACAC | |
| | GGAGCCCAGCCCUCCCCAUGGAGCCAGC | |
| | UCCCUCUAUUUAUGUUUGCACUUGUGA | |
| | UUAUUUAUUAUUUAUUUAUUAUUUAUU | |
| | UAUUUACAGAUGAAUGUAUUUAUUUGG | |
| | GAGACCGGGGUAUCCUGGGGGACCCAAU | |
| | GUAGGAGCUGCCUUGGCUCAGACAUGU | |
| | UUUCCGUGAAAACGGAGCUGAACAAUA | |
| | GGCUGUUCCCAUGUAGCCCCCUGGCCUC | |
| | UGUGCCUUCUUUUGAUUAUGUUUUUUA | |
| | AAAUAUUUAUCUGAUUAAGUUGUCUAA | |
| | ACAAUGCUGAUUUGGUGACCAACUGUC | |
| | ACUCAUUGCUGAGCCUCUGCUCCCCAGG | |
| | GGAGUUGUGUCUGUAAUCGCCCUACUA | |
| | UUCAGUGGCGAGAAAUAAAGUUUGCUU | |
| | AGAAAAGAA | |
| 264 | UCCUCAGCCUCUUCUCCUU | 183 |
| 984 | GGGUCGGAACCCAAGCUUA | 184 |
| 430 | GCCUGUAGCCCAUGUUGUA | 185 |
| 558 | GCCUGUACCUCAUCUACUCUU | 186 |
| 270 | GCCUCUUCUCCUUCCUGAUCGUGdGdC | 187 |
| 315 | GCCUGCUGCACUUUGGAGUGAUCdGdG | 188 |
| 612 | CCCAUGUGCUCCUCACCCACACCdAT | 189 |
| 564 | ACCUCAUCUACUCCCAGGUCCUCdTdT | 190 |
| 787 | CCGACUCAGCGCUGAGAUCAA | 191 |

The introduction of substituted and modified nucleotides into dsRNA molecules of this disclosure provides a powerful tool in overcoming potential limitations of in vivo stability and bioavailability inherent to native RNA molecules (i.e., having standard nucleotides) that are delivered exogenously. For example, the use of dsRNA molecules of this disclosure can enable a lower dose of a particular nucleic acid molecule for a given therapeutic effect (e.g., reducing or silencing gene expression to treat or prevent disease) since dsRNA molecules of this disclosure tend to have a longer half-life in serum. Furthermore, certain substitutions and modifications can improve the bioavailability of dsRNA by targeting particular cells or tissues or improving cellular uptake of the dsRNA molecules. Therefore, even if the activity of a dsRNA molecule of this disclosure is reduced as compared to a native (unsubstituted and unmodified) RNA molecule, the overall activity or potency of the substituted or modified dsRNA molecule can be greater than that of the native RNA molecule due to improved stability or delivery of the molecule. Unlike native dsRNA, substituted or modified dsRNA can also reduce off-target effects and minimize the possibility of activating the interferon response in, for example, humans.

In certain embodiments, a drtRNA molecules comprise ribonucleotides at about 1% to about 25% or at about 5% to about 50% or at about 50% to about 100% of the nucleotide positions.

Substituted or modified nucleotides present in dsRNA molecules, such as in the antisense strand, but also optionally in the sense or both the antisense and sense strands, comprise modified or substituted nucleotides according to this disclosure having properties or characteristics similar to natural or standard ribonucleotides (but providing enhanced properties in a biological system). For example, this disclosure features dsRNA molecules including nucleotides having a Northern conformation (e.g., Northern pseudorotation cycle, see for example Saenger, Principles of Nucleic Acid Structure, Springer-Verlag ed., 1984). As such, chemically modified nucleotides present in dsRNA molecules for use in the methods of this disclosure, such as in the antisense strand, but also optionally in the sense or both the antisense and sense strands, are resistant to nuclease degradation while at the same time maintaining the capacity to mediate RNAi. Exemplary nucleotides having a Northern configuration include locked nucleic acid (LNA) nucleotides (e.g., 2′-O, 4′-C-methylene-(D-ribofuranosyl) nucleotides); 2′-methoxyethyl (MOE) nucleotides; 2′-methyl-thio-ethyl, 2′-deoxy-2′-fluoro nucleotides. 2′-deoxy-2′-chloro nucleotides, 2′-azido nucleotides, 5-methyluridines, or 2′-O-methyl nucleotides.

Another aspect of the disclosure is a drtRNA comprising an acyclic nucleotide monomer. In a preferred embodiment, the acyclic nucleotide monomer is a 2′-3′-seco-nucleotide monomer. Preferably, the acyclic nucleotide monomer is selected from the group consisting of monomer E, F, G, H, I or J (see below).

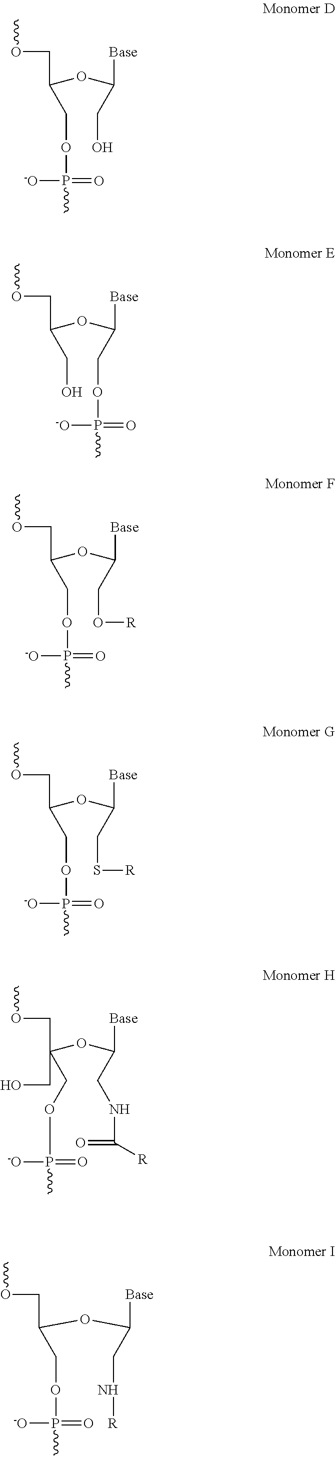

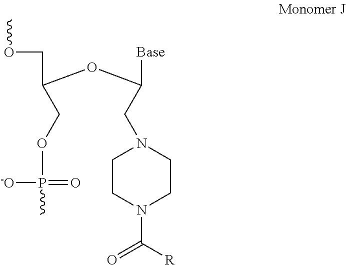

Other examples of acyclic nucleotide monomers are described, for example, in PCT patent application PCT/US2008/64417, hereby incorporated by reference in their entirety.

In any of the embodiments herein, the instant disclosure provides a nucleotide having the following formula:

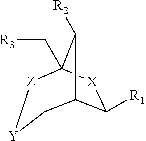

wherein, X is O or CH₂, Y is O, and Z is CH₂;

R₁ is selected from the group consisting of adenine, cytosine, guanine, hypoxanthine, uracil, thymine, and a heterocycle wherein the heterocycle is selected from the group consisting of a substituted 1,3-diazine, unsubstituted 1,3-diazine, and an unsubstituted 7H imidazo[4,5]1,3 diazine; and

R₂, R₃ are independently selected from a group consisting of H, OH, DMTO, TBDMSO, BnO, THPO, AcO, BzO, OP(NiPr₂)O(CH₂)₂CN, OPO₃H, diphosphate, and triphosphate, wherein R₂ and R₃ together may be PhCHO₂, TIPDSO₂ or DTBSO₂. Other examples of locked nucleic acids are described, for example, in U.S. Pat. Nos. 5,681,940; 5,712,378; 6,191,266; 6,403,566; 6,479,463; and 6,509,320, hereby incorporated by reference in their entirety.

As described herein, the first and second strands of a dsRNA molecule or analog thereof provided by this disclosure can anneal or hybridize together (i.e., due to complementarity between the strands) to form a double-stranded region having a length of about 10 to about 40 base pairs. In some embodiments, the dsRNA has a double-stranded region ranging in length from about 15 to about 29 base pairs or about 19 to about 23 base pairs or about 19 to about 21 base pairs, which can be loaded into RISC. In related embodiments, a dsRNA that is loaded into RISC will have at least one 5-methyluridine:2,6-diaminopurine base pair within the first eight nucleotides of the 5′-end of the antisense strand (also known as a “seed region” of an antisense strand of a dsRNA). In other embodiments, the dsRNA has a double-stranded region ranging in length from about 29 to about 40 base pairs or about 30 to about 35 base pairs, which forms a Dicer substrate. In related embodiments, a dsRNA that is a Dicer substrate will have at least one 5-methyluridine:2,6-diaminopurien base pair within the seed region of the dsRNA products resulting from Dicer cleavage (i.e., the Dicer products that will load into RISC). For example, a dsRNA forming a double stranded region of 40 base pairs can be a Dicer substrate that can cleave the dsRNA into two dsRNAs of 21 base pairs (formerly only having a 5′-end) and 19 base pairs (formerly having only a 3′-end)—the first eight nucleotides at the 5′-end of the antisense strand of the 21 base pair fragment will have at least one 5-methyluridine:2,6-diaminopurine base pair (i.e., the seed region of the dsRNA to be loaded in RISC) and the first eight nucleotides at the 5′-end of the antisense strand of the 19 base pair fragment will have at least one 5-methyluridine:2,6-diaminopurine base pair (i.e., the 5′-end of which was formerly an internal sequence of the parent 40 base pair dsRNA). In other embodiments, the two strands of a dsRNA molecule of this disclosure may optionally be covalently linked together by nucleotide or non-nucleotide linker molecules, or one of the strands may be separated by a nick or gap (e.g., forming an A:B1B2 configuration as described herein).

In certain embodiments, a dsRNA molecule or analog thereof for use in the methods of this disclosure has an overhang of one to four nucleotides on one or both 3′-ends of the dsRNA, such as an overhang comprising a deoxyribonucleotide or two deoxyribonucleotides (e.g., thymidine or adenine). In any of the embodiments of dsRNA molecules described herein, the 3′-terminal nucleotide overhangs of a dsRNA molecule for use in the methods of this disclosure can comprise ribonucleotides or deoxyribonucleotides that are chemically-modified at a nucleic acid sugar, base, or backbone. In any of the embodiments of dsRNA molecules described herein, the 3′-terminal nucleotide overhangs can comprise one or more universal base ribonucleotides. In any of the embodiments of dsRNA molecules described herein, the 3′-terminal nucleotide overhangs can comprise one or more acyclic nucleotides. In some embodiments, a dsRNA molecule or analog thereof for use in the methods of this disclosure has a blunt end at one or both ends of the dsRNA. In any of the embodiments of dsRNA molecules described herein, the dsRNA can further comprise a terminal phosphate group, such as a 35-phosphate (see, Martinez et al., Cell. 110:563-574, 2002; and Schwarz et al., Molec. Cell 10:537-568, 2002) or a 5′,3″diphosphate.

As set forth herein, the terminal structure of dsRNAs that decrease expression of a target gene by RNAi for use in the methods of this disclosure may either have one or more blunt end or one or more overhang. In certain embodiments, a dsRNA molecule overhang may be at the 3′-end or the 5′-end. The total length of dsRNAs having overhangs is expressed as the sum of the length of the paired double-stranded portion and of the overhanging nucleotides. For example, if a 19 base pair dsRNA has a two nucleotide overhang at both ends, the total length is expressed as 21-mer. Furthermore, since the overhanging sequence may have low specificity to a target gene, it is not necessarily complementary (antisense) or identical (sense) to the target gene sequence. In further embodiments, a dsRNA of this disclosure that decreases expression of a target gene by RNAi may further comprise a low molecular weight structure (for example, a natural RNA molecule such as a tRNA, rRNA or viral RNA, or an artificial RNA molecule) at, for example, one or more overhanging portion of the dsRNA.

In further embodiments, a dsRNA molecule that decreases expression of a target gene by RNAi for use in the methods according to the instant disclosure further comprises a terminal cap substituent at one or both ends of the first strand or second strand, such as an alkyl, abasic, deoxy abasic, glyceryl, dinucleotide, acyclic nucleotide, inverted deoxynucleotide moiety, or any combination thereof. In certain embodiments, at least one or two 5′-terminal ribonucleotides of the sense strand within the double-stranded region have a 2′-sugar substitution. In certain other embodiments, at least one or two 5′-terminal ribonucleotides of the antisense strand within the double-stranded region have a 2′-sugar substitution. In certain embodiments, at least one or two 5′-terminal ribonucleotides of the sense strand and the antisense strand within the double-stranded region have a 2′-sugar substitution.

In yet other embodiments, a dsRNA molecule that decreases expression of a target gene by RNAi for use in the methods according to the instant disclosure further comprises at least one modified intemucleoside linkage, such as a phosphorothioate, chiral phosphorothioate, phosphorodithioate, phosphotriester, aminoalkylphosphotriester, methyl phosphonate, alkyl phosphonate, 3′-alkylene phosphonate, 5′-alkylene phosphonate, chiral phosphonate, phosphonoacetate, thiophosphonoacetate, phosphinate, phosphoramidate, 3′-amino phosphoramidate, aminoalkylphosphoramidate, thionophosphoramidate, thionoalkylphosphonate, thionoalkylphosphotriester, selenophosphate, boranophosphate linkage, or any combination thereof.

A modified internucleotide linkage, as described herein, can be present in one or both strands of a dsRNA molecule for use in the methods of this disclosure, for example, in the sense strand, the antisense strand, or both strands. The dsRNA molecules of this disclosure can comprise one or more modified internucleotide linkages at the 3′-end, the 5′-end, or both of the 3′- and 5′-ends of the sense strand or the antisense strand or both strands. In one embodiment, a dsRNA molecule capable of decreasing expression of a target gene by RNAi has one modified internucleotide linkage at the 3′-end, such as a phosphorothioate linkage. For example, this disclosure provides a dsRNA molecule capable of decreasing expression of a target gene by RNAi having about 1 to about 8 or more phosphorothioate internucleotide linkages in one dsRNA strand. In yet another embodiment, this disclosure provides a dsRNA molecule capable of decreasing expression of a target gene by RNAi having about 1 to about 8 or more phosphorothioate internucleotide linkages in both dsRNA strands. In other embodiments, an exemplary dsRNA molecule of this disclosure can comprise from about 1 to about 5 or more consecutive phosphorothioate internucleotide linkages at the 5′-end of the sense strand, the antisense strand, or both strands. In another example, an exemplary dsRNA molecule of this disclosure can comprise one or more pyrimidine phosphorothioate internucleotide linkages in the sense strand, the antisense strand, or both strands. In yet another example, an exemplary dsRNA molecule of this disclosure can comprise one or more purine phosphorothioate internucleotide linkages in the sense strand, the antisense strand, or both strands.

In still further embodiments, a dsRNA for use in the methods according to the instant disclosure further comprises a terminal cap substituent on one or both ends of the first strand or second strand, such as an alkyl, abasic, deoxy abasic, glyceryl, dinucleotide, acyclic nucleotide, inverted deoxynucleotide moiety, or any combination thereof. In further embodiments, one or more internucleoside linkage can be optionally modified. For example, a dsRNA according to the instant disclosure wherein at least one internucleoside linkage is modified to a phosphorothioate, chiral phosphorothioate, phosphorodithioate, phosphotriester, aminoalkylphosphotriester, methyl phosphonate, alkyl phosphonate, 3′-alkylene phosphonate, 5′-alkylene phosphonate, chiral phosphonate, phosphonoacetate, thiophosphonoacetate, phosphinate, phosphoramidate, 3′-amino phosphoramidate, aminoalkylphosphoramidate, thionophosphoramidate, thionoalkylphosphonate, thionoalkylphosphotriester, selenophosphate, boranophosphate linkage, or any combination thereof.

In certain aspects, a nicked or gapped dsRNA molecule (ndsRNA or gdsRNA, respectively) that decreases expression of a target gene by RNAi, comprising a first strand that is complementary to the target gene mRNA and two or more second strands that are complementary to the first strand, wherein the first and at least two of the second strands form a non-overlapping double-stranded region of about 10 to about 40 base pairs. Any of the aforementioned substitutions or modifications is applicable to this embodiment as well.

In further embodiments, the instant disclosure provides a method for activating target gene-specific RNA interference (RNAi) by administering a double-stranded ribonucleic acid (dsRNA) molecule that decreases expression of a target gene by RNAi to a cell expressing the target gene, wherein the dsRNA comprises two or more first strands that are complementary to the target gene mRNA and a second strand that is complementary to the two or more first strands, wherein at least one of the first strands and the second strand form a double-stranded region of about 10 to about 40 base pairs, wherein with the double-stranded region comprises at least one base pair comprising a 5-methyluridine base paired with a 2,6-diaminopurine, and wherein the dsRNA has a minimal off-target effect. In another embodiment, the dsRNA molecule can comprise a first strand (B1), a second first strand (B2) and a second strand (A), wherein the double-stranded region formed by the annealed B1 and A strands is distinct from and non-overlapping with the double-stranded region formed by the annealed B2 and A strands. For example, there are three strands (two first strands that together make up the antisense strand and the second strand that is the sense strand) that anneal to form a dsRNA molecule with one strand (antisense) having a nick (i.e., a break in the “first strand”—that is, lacking an internucleoside linkage between the two first strands).

In some embodiments, the double-stranded region formed by the annealed B1 and A strands is separated by a gap from the double-stranded region formed by the annealed B2 and A strands, wherein the gap is at least one unpaired nucleotide in the A strand that is positioned between the A:B1 double-stranded region and the A:B2 double-stranded region. In certain embodiments, the gap can be from about one to about ten nucleotides in length. In further embodiments, the dsRNA can have an overhang of one to four nucleotides on one or both 3′-end and the gap will be distinct from any one or more overhang at the 3′-end of one or more of the A, B1, or B2 strands. In still further embodiments, the A strand is about 10 to about 40 nucleotides in length, and the B1 and B2 strands are each, individually, about 5 to about 20 nucleotides, wherein the combined length of the B1 and B2 strands ranges from about 15 nucleotides to about 40 nucleotides. In any of these embodiments, the double-stranded region contains at least one, two, three, four, five, or more base pairs (including 6, 7, 8, 9, 10, 11, 12, 13, 14, 15, 16, 17, 18, 19, 20 or more base pairs), each base pair comprising a 5-methyluridine base paired with a 2,6-diaminopurine. In any of these embodiments, the dsRNA can further include a 2′-sugar substitution as described herein.

As described herein, the first and second strands of an ndsRNA or gdsRNA molecule or analog thereof provided by this disclosure can anneal or hybridize together (i.e., due to complementarity between the strands) to form a double-stranded region having a length of about 10 to about 40 base pairs. In some embodiments, the dsRNA has a double-stranded region ranging in length from about 15 to about 29 base pairs or about 19 to about 23 base pairs. In other embodiments, the dsRNA has a double-stranded region ranging in length from about 29 to about 40 base pairs or about 30 to about 35 base pairs. In certain embodiments, the dsRNA molecule or analog thereof has an overhang of one to four nucleotides on one or both 3′-ends of the dsRNA, such as an overhang comprising a deoxyribonucleotide or two deoxyribonucleotides (e.g., thymidine, adenosine, guanosine, and cytidine). In some embodiments, a dsRNA molecule or analog thereof has a blunt end at one or both ends. In certain embodiments, the 5′-end of the first or second or both strands is phosphorylated.

In addition, the terminal structure of the dsRNAs of this disclosure may have a stem-loop structure in which ends of one side of the dsRNA molecule are connected by a linker nucleic acid, e.g., a linker RNA. The length of the double-stranded region (stem-loop portion) can be, for example, about 15 to about 49 bp, or about 15 to about 35 bp, or about 21 bp to about 30 bp long. Alternatively, the length of the double-stranded region that is a final transcription product of dsRNAs to be expressed in a target cell may be, for example, approximately 15 to about 49 bp, about 15 to about 35 bp, or about 21 to about 30 bp long. When linker segments are employed, there is no particular limitation in the length of the linker as long as it does not hinder pairing of the stem portion. For example, for stable pairing of the stem portion and suppression of recombination between DNAs coding for this portion, the linker portion may have a clover-leaf tRNA structure. Even if the linker has a length that would hinder pairing of the stem portion, it is possible, for example, to construct the linker portion to include introns so that the introns are excised during processing of a precursor RNA into mature RNA, thereby allowing pairing of the stem portion. In the case of a stem-loop dsRNA, either end (head or tail) of RNA with no loop structure may have a low molecular weight RNA. As described above, these low molecular weight RNAs may include a natural RNA molecule, such as tRNA, rRNA or viral RNA, or an artificial RNA molecule.

An dsRNA molecule may be comprised of a circular nucleic acid molecule, wherein the dsRNA is about 38 to about 70 nucleotides in length having from about 18 to about 23 base pairs (e.g., about 19 to about 21) wherein the circular oligonucleotide forms a dumbbell shaped structure having about 19 base pairs and 2 loops. In certain embodiments, a circular dsRNA molecule contains two loop motifs, wherein one or both loop portions of the dsRNA molecule is biodegradable. For example, a circular dsRNA molecule of this disclosure is designed such that degradation of the loop portions of the dsRNA molecule in vivo can generate a double-stranded dsRNA molecule with 3′-terminal overhangs, such as 3′-terminal nucleotide overhangs comprising from about 1 to about 4 (unpaired) nucleotides.

In another embodiment, a conjugate molecule can be optionally attached to a dsRNA or analog thereof that decreases expression of a target gene by RNAi. For example, such conjugate molecules may be polyethylene glycol, human serum albumin, or a ligand for a cellular receptor that can, for example, mediate cellular uptake. Examples of specific conjugate molecules contemplated by the instant disclosure that can be attached to a dsRNA or analog thereof of this disclosure are described in Vargeese et al., U.S. Patent Application Publication No. 2003/0130186, published Jul. 10, 2003, and U.S. Patent Application Publication No. 2004/0110296, published Jun. 10, 2004. In another embodiment, a conjugate molecule is covalently attached to a dsRNA or analog thereof that decreases expression of a target gene by RNAi via a biodegradable linker. In certain embodiments, a conjugate molecule can be attached at the 3′-end of either the sense strand, the antisense strand, or both strands of a dsRNA molecule provided herein. In another embodiment, a conjugate molecule can be attached at the 5′-end of either the sense strand, the antisense strand, or both strands of the dsRNA or analog thereof. In yet another embodiment, a conjugate molecule is attached both the 3′-end and 5′-end of either the sense strand, the antisense strand, or both strands of a dsRNA molecule, or any combination thereof.

In further embodiments, a conjugate molecule of this disclosure comprises a molecule that facilitates delivery of a dsRNA or analog thereof into a biological system, such as a cell. The type of conjugates used and the extent of conjugation of dsRNA of this disclosure can be evaluated for improved pharmacokinetic profiles, bioavailability, or stability while at the same time tested for the ability to mediate RNAi. As such, one skilled in the art can screen dsRNA or analogs thereof having various conjugates to determine whether the dsRNA-conjugate complex possesses improved properties while maintaining the ability to mediate RNAi, for example, in animal models described herein and generally known in the art.

A drtRNA according to this disclosure will often increase resistance to enzymatic degradation, such as exonucleolytic degradation, including 5′-exonucleolytic or 3′-exonucleolytic degradation. As such, the drtRNAs described herein will exhibit significant resistance to enzymatic degradation compared to a corresponding dsRNA having standard nucleotides, and will thereby possess greater stability, increased half-life, and greater bioavailability in physiological environments (e.g., when introduced into a eukaryotic target cell). In addition to increasing resistance to exonucleolytic degradation, the presence of a 5-methyluridine:2,6-diaminopurine base pair in the double-stranded region of a dsRNA will render the dsRNAs more resistant to other enzymatic or chemical degradation processes, and thus more stable and bioavailable than otherwise identical dsRNAs that do not include the 5-methyluridine:2,6-diaminopurine base pair. In related aspects of this disclosure, drtRNAs described herein will often have improved stability for use within research, diagnostic and treatment methods wherein the drtRNA is contacted with a biological sample, for example, a mammalian cell, intracellular compartment, serum or other extracellular fluid, tissue, or other in vitro or in vivo physiological compartment or environment. In one embodiment, diagnosis is performed on an isolated biological sample. In another embodiment, the diagnostic method is performed in vitro. In a further embodiment, the diagnostic method is not performed (directly) on a human or animal body.

In another aspect of this disclosure, drtRNAs described herein will have reduced “off-target effects” when they are contacted with a biological sample (e.g., when introduced into a target eukaryotic cell having specific, and non-specific mRNA species present as potential specific and non-specific targets). In related embodiments, drtRNAs according to this disclosure are employed in methods of gene silencing, wherein the drtRNAs exhibit reduced or eliminated off-target effects compared to a corresponding, dsRNAs not having a 5-methyluridine:2,6-diaminopurine base pair, e.g., as determined by non-specific inhibition (or activation) of genes in addition to a target (i.e., homologous or cognate) gene in a cell or other biological sample to which the drtRNA is exposed under conditions that allow for gene silencing activity to be detected.

In yet another aspect of this disclosure, the drtRNA described herein will have reduced interferon activation by the dsRNA molecule when the dsRNA is contacted with a biological sample, e.g., when introduced into a eukaryotic cell.

In still another aspect, this disclosure provides methods for inhibiting expression of a target gene in a eukaryotic cell. The method includes introducing a drtRNA of this disclosure into the cell, and maintaining the cell for a time sufficient to allow the drtRNA to mediate down regulation of gene expression, which can include degradation of an mRNA transcript of a target gene. In the case of mammalian subjects, those subjects amenable for treatment using the compositions and methods of this disclosure will include human and other mammalian subjects suffering from one or more diseases or conditions mediated, at least in part, by over expression of a target gene. In exemplary embodiments, the methods and compositions of this disclosure are employed to treat a disease or condition mediated by over expression of one or more target genes/proteins, for example, a hyperproliferative, metabolic syndrome, neural, cardiac, immune, or inflammatory disease or disorder.

In further embodiments, dsRNAs of this disclosure can comprise a sense (second) strand that is homologous or corresponds to a sequence of a target gene and an antisense (first) strand that is complementary to the sense strand and a sequence of the target gene (e.g., TNF, TNFR, VEGFR).

By way of background, within the silencing complex, the dsRNA molecule is positioned so that the target RNAs can bump into it. The RISC will encounter thousands of different RNAs that are in a typical cell at any given moment. But, the dsRNA loaded in RISC will adhere well only to a target RNA that has close complementarity with the antisense strand of the dsRNA. So, unlike an interferon response to a viral infection, the silencing complex is highly selective in choosing its target RNAs. The RISC cleaves the captured RNA strand in two and releases the two pieces of the RNA (now rendered incapable of directing protein synthesis) and moves on. The RISC itself stays intact and is capable of finding and cleaving other RNA molecules.

In certain aspects, the instant disclosure provides method for activating target gene-specific RNA interference (RNAi), comprising administering a double-stranded ribonucleic acid (dsRNA) molecule that decreases expression of a target gene by RNAi to a cell expressing the target gene, wherein the dsRNA comprises a first strand that is complementary to the target gene mRNA and a second strand that is complementary to the first strand, wherein the first and second strands form a double-stranded region of about 15 to about 29 base pairs for association with RISC or of about 29 to about 40 base pairs to form a Dicer substrate, wherein the dsRNA has at least one base pair comprising a 5-methyluridine base paired with a 2,6-diaminopurine, and wherein the dsRNA has a minimal off-target effect.

### Synthesis of Nucleic Acid Molecules

Exemplary molecules of the instant disclosure are recombinantly produced, chemically synthesized, or a combination thereof. Oligonucleotides (e.g., certain modified oligonucleotides or portions of oligonucleotides lacking ribonucleotides) are synthesized using protocols known in the art, for example as described in Caruthers et al., Methods in Enzymol. 211:3-19, 1992; Thompson et al., PCT Publication No. WO 99/54459, Wincott et al., 1995, Nucleic Acids Res., 23:2677-2684, Wincott et al., Methods Mol. Bio. 74:59, 1997; Brennan et al., Biotechnol Bioeng. 61:33-45, 1998; and Brennan, U.S. Pat. No. 6,001,311. Synthesis of RNA, including certain dsRNA molecules and analogs thereof of this disclosure, can be made using the procedure as described in Usman et al., J. Am. Chem. Soc. 109:7845, 1987; Scaringe et al., Nucleic Acids Res. 18:5433, 1990; and Wincott et al., Nucleic Acids Res. 23:2677-2684, 1995; Wincott et al., Methods Mol. Bio. 74:59, 1997.

In certain embodiments, the nucleic acid molecules of the present disclosure can be synthesized separately and joined together post-synthetically, for example, by ligation (Moore et al., Science 256:9923, 1992; Draper et al., PCT Publication No. WO 93/23569; Shabarova et al., Nucleic Acids Res. 19:4247, 1991; Bellon et al., Nucleosides & Nucleotides 16:951, 1997; Bellon et al., Bioconjugate Chem. 8:204, 1997), or by hybridization following synthesis or deprotection.

In further embodiments, dsRNAs of this disclosure that decrease expression of a target gene by RNAi can be made as single or multiple transcription products expressed by a polynucleotide vector encoding the single or multiple dsRNAs and directing their expression within host cells. In these embodiments the double-stranded portion of a final transcription product of the dsRNAs to be expressed within the target cell can be, for example, 10 to 49 bp long (or 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49 base pairs long), 15 to 35 bp long (or 15, 16, 17, 18, 19, 20, 21, 22, 23, 24, 25, 26, 27, 28, 29, 30, 31, 32, 33, 34, or 35 base pairs long), or about 21 to 30 bp long (or 21, 22, 23, 24, 25, 26, 27, 28, 29, or 30 base pairs long). Within exemplary embodiments, double-stranded portions of dsRNAs, in which two strands pair up, are not limited to completely paired nucleotide segments, and may contain non-pairing portions due to mismatch (the corresponding nucleotides are not complementary), bulge (lacking in the corresponding complementary nucleotide on one strand), overhang, and the like. Non-pairing portions can be contained to the extent that they do not interfere with dsRNA formation. In more detailed embodiments, a “bulge” may comprise 1 to 2 non-pairing nucleotides, and the double-stranded region of dsRNAs in which two strands pair up may contain from about 1 to 7 (or 1, 2, 3, 4, 5, 6, or 7), or about 1 to 5 (or 1, 2, 3, 4, or 5) bulges. In addition, “mismatch” portions contained in the double-stranded region of dsRNAs may be present in numbers from about 1 to 7 (or 1, 2, 3, 4, 5, 6, or 7), or about 1 to 5 (or 1, 2, 3, 4, or 5). In other embodiments, the double-stranded region of dsRNAs of this disclosure may contain both bulge and mismatched portions in the approximate numerical ranges specified herein.

A dsRNA or analog thereof of this disclosure may be further comprised of a nucleotide, non-nucleotide, or mixed nucleotide/non-nucleotide linker that joins the sense region of the dsRNA to the antisense region of the dsRNA. In one embodiment, a nucleotide linker can be a linker of more than about 2 nucleotides length up to about 10 nucleotides in length ( or 2, 3, 4, 5, 6, 7, 8, 9, or 10). In another embodiment, the nucleotide linker can be a nucleic acid aptamer. By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule wherein the target molecule does not naturally bind to a nucleic acid. The target molecule can be any molecule of interest. For example, the aptamer can be used to bind to a ligand-binding domain of a protein, thereby preventing interaction of the naturally occurring ligand with the protein. This is a non-limiting example and those in the art will recognize that other embodiments can be readily generated using techniques generally known in the art (see, for example, Gold et al., Annu. Rev. Biochem. 64:763, 1995; Brody and Gold, J. Biotechnol. 74:5, 2000; Sun, Curr. Opin. Mol. Ther. 2:100, 2000; Kusser, J. Biotechnol. 74:27, 2000; Hermann and Patel, Science 287:820, 2000; and Jayasena, Clinical Chem. 45:1628, 1999).

A non-nucleotide linker may be comprised of an abasic nucleotide, polyether, polyamine, polyamide, peptide, carbohydrate, lipid, polyhydrocarbon, or other polymeric compounds (e.g., polyethylene glycols such as those having between 2 and 100 ethylene glycol units). Specific examples include those described by Seela and Kaiser, Nucleic Acids Res. 18:6353, 1990, and Nucleic Acids Res. 15:3113, 1987; Cload and Schepartz, J. Am. Chem. Soc. 113:6324, 1991; Richardson and Schepartz, J. Am. Chem. Soc. 113:5109, 1991; Ma et al., Nucleic Acids Res. 21:2585, 1993, and Biochemistry 32:1751, 1993; Durand et al., Nucleic Acids Res. 18:6353, 1990; McCurdy et al., Nucleosides & Nucleotides 10:287, 1991; Jaschke et al., Tetrahedron Lett. 34:301, 1993; Ono et al., Biochemistry 30:9914, 1991; Arnold et al., PCT Publication No. WO 89/02439; Usman et al., PCT Publication No. WO 95/06731; Dudycz et al., PCT Publication No. WO 95/11910 and Ferentz and Verdine, J. Am. Chem. Soc. 113:4000, 1991. The synthesis of a dsRNA molecule of this disclosure, which can be further modified, comprises: (a) synthesis of two complementary strands of the dsRNA molecule; and (b) annealing the two complementary strands together under conditions suitable to obtain a dsRNA molecule. In another embodiment, synthesis of the two complementary strands of a dsRNA molecule is by solid phase oligonucleotide synthesis. In yet another embodiment, synthesis of the two complementary strands of a dsRNA molecule is by solid phase tandem oligonucleotide synthesis.

Chemically synthesizing nucleic acid molecules with substitutions or modifications (base, sugar or phosphate) can prevent their degradation by serum ribonucleases, which can increase their potency. See e.g., Eckstein et al., PCT Publication No. WO 92/07065; Perrault et al., Nature 344:565, 1990; Pieken et al., Science 253:314, 1991; Usman and Cedergren, Trends in Biochem. Sci. 17:334, 1992; Usman et al., PCT Publication No. WO 93/15187; and Rossi et al., PCT Publication No. WO 91/03162; Sproat, U.S. Pat. No. 5,334,711; Gold et al., U.S. Pat. No. 6,300,074. All of the above references describe various chemical modifications that can be made to the base, phosphate or sugar moieties of the nucleic acid molecules described herein.

There are several examples in the art describing sugar, base and phosphate modifications that can be introduced into nucleic acid molecules with significant enhancement in their nuclease stability and efficacy. For example, oligonucleotides are modified to enhance stability or enhance biological activity by modification with nuclease resistant groups, for example, 2′-amino, 2′-C-allyl, 2′-fluoro, 2′-O-methyl, 2′-O-allyl, 2′-H, nucleotide base modifications. For a review see Usman and Cedergren, TIBS 17:34, 1992; Usman et al., Nucleic Acids Symp. Ser. 31:163, 1994; Burgin et al., Biochemistry 35:14090, 1996. Sugar modification of nucleic acid molecules have been extensively described in the art (see Eckstein et al., PCT Publication No. WO 92/07065; Perrault et al., Nature 344:565-568, 1990; Pieken et al., Science 253:314-317, 1991; Usman and Cedergren, Trends in Biochem. Sci. 17:334-339, 1992; Usman et al., PCT Publication No. WO 93/15187; Sproat, U.S. Pat. No. 5,334,711 and Beigelman et al., J. Biol. Chem. 270:25702, 1995; Beigelman et al., PCT Publication No. WO 97/26270; Beigelman et al., U.S. Pat. No. 5,716,824; Usman et al., U.S. Pat. No. 5,627,053; Woolf et al., PCT Publication No. WO 98/13526; Thompson et al., Karpeisky et al., Tetrahedron Lett. 39:1131, 1998; Eamshaw and Gait, Biopolymers (Nucleic Acid Sciences) 48:39-55, 1998; Verma and Eckstein, Annu. Rev. Biochem. 67:99-134, 1998; and Burlina et al., Bioorg. Med. Chem. 5:1999-2010, 1997. Such publications describe general methods and strategies to determine the location of incorporation of sugar, base or phosphate modifications and the like into nucleic acid molecules without modulating catalysis. In view of such teachings, similar modifications can be used as described herein to modify the dsRNA molecules of the instant disclosure so long as the ability of the dsRNA molecule to promote RNAi in cells is not significantly inhibited.

In one embodiment, this disclosure features substituted or modified dsRNA molecules, such as phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, or alkylsilyl, substitutions. For a review of oligonucleotide backbone modifications, see Hunziker and Leumann, Nucleic Acid Analogues: Synthesis and Properties, in Modern Synthetic Methods, VCH, 331-417, 1995; and Mesmaeker et al., “Novel Backbone Replacements for Oligonucleotides, in Carbohydrate Modifications in Antisense Research,” ACS, 24-39, 1994.

Methods for Selecting dsRNA and Analogs Thereof Specific for a Target Gene

As indicated above, the present disclosure also provides methods for selecting dsRNA and analogs thereof that are capable of specifically binding to a target gene while being incapable of specifically binding or minimally binding to non-target genes. The selection process disclosed herein is useful, for example, in eliminating dsRNAs analogs that are cytotoxic due to non-specific binding to, and subsequent degradation of, one or more non-target genes. It will be understood that methods of the present disclosure do not require a priori knowledge of the nucleotide sequence of every possible gene variant targeted by the dsRNA or analog thereof. In one embodiment, the nucleotide sequence of the dsRNA is selected from a conserved region or consensus sequence of a target gene.

In certain embodiments, methods are provided for selecting one or more dsRNA molecule that decreases expression of a target gene by RNAi, comprising a first strand that is complementary to the target mRNA (e.g., target sequences provided by the accession numbers of Table A) and a second strand that is complementary to the first strand, wherein the first and second strands form a double-stranded region of about 10 to about 40 base pairs, and wherein the double-stranded region has at least one base pair comprising a 5-methyluridine base paired with a 2,6-diaminopurine, which methods employ “off-target” profiling whereby one or more dsRNA provided herein is contacted with a cell, either in vivo or in vitro, and total target mRNA is collected for use in probing a microarray comprising oligonucleotides having one or more nucleotide sequence from a panel of known genes, including non-target genes. The “off-target” profile of the dsRNA provided herein is quantified by determining the number of non-target genes having reduced expression levels in the presence of the candidate dsRNAs. The existence of “off-target” binding indicates a dsRNA provided herein that is capable of binding one or more non-target gene messages. In certain embodiments, a dsRNA as provided herein (e.g., based on sequences provided by the accession numbers of Table A) applicable to therapeutic use will exhibit minimal “off-target” binding and optionally greater stability, minimal interferon response, greater potency, or any combination thereof.

Still further embodiments provide methods for selecting more efficacious dsRNA by using one or more reporter gene constructs comprising a constitutive promoter, such as a cytomegalovirus (CMV) or phosphoglycerate kinase (PGK) promoter, operably fused to and capable of altering the expression of one or more reporter genes (such as a luciferase, chloramphenicol (CAT), or β-galactosidase), which, in turn, is operably fused in-frame with a dsRNA (such as one having a length from about 10 to about 40 nucleotides or from about 15 nucleotides to about 29 nucleotides, or from about 29 nucleotides to 40 nucleotides) that contains a target sequence as provided herein. Individual reporter gene expression constructs may be co-transfected with one or more dsRNA. The capacity of a given dsRNA to reduce the expression level of a target gene may be determined by comparing the measured reporter gene activity in cells transfected with or without a dsRNA of interest.

Methods are provided for selecting one or more modified dsRNA molecule(s) that involve predicting the stability of a dsRNA duplex. In some embodiments, such a prediction is achieved by employing a theoretical melting curve wherein a higher theoretical melting curve indicates an increase in dsRNA duplex stability and a concomitant decrease in cytotoxic effects. Alternatively, stability of a dsRNA duplex may be determined empirically by measuring the hybridization of a single RNA analog strand as described herein to a complementary target gene within, for example, a polynucleotide array. The melting temperature (i.e., the Tₘ value) for each modified RNA and complementary RNA immobilized on the array can be determined and, from this Tₘ value, the relative stability of the modified RNA pairing with a complementary RNA molecule determined.

For example, Kawase et al. (Nucleic Acids Res. 14:7727-7736, 1986) have described an analysis of the nucleotide-pairing properties of Di (inosine) to A, C, G , and T, which was achieved by measuring the hybridization of oligonucleotides (ODNs) with Di in various positions to complementary sets of ODNs made as an array. The relative strength of nucleotide-pairing is I-C>I-A>I-G˜I-T. Generally, Di containing duplexes showed lower Tₘ values when compared to the corresponding WC nucleotide pair. The stabilization of Di by pairing was in order of Dc>Da>Dg>Dt>Du (see Table 2A).

TABLE 2A

Stability of Inosine Binding Compared to Standard
Nucleotide Binding

d(GGAAAAXAAAAGG) (SEQ ID NO: 220)
d(CCTTTTYTTTTCC) (SEQ ID NO: 221)

| Duplex X/Y | | Corresponding WT | | Corresponding WT | |
|---|---|---|---|---|---|
| nucleotide | Tₘ | sequence wherein | Tₘ | sequence wherein | |
| pair | (° C.) | X/Y are | (° C.) | X/Y are | Tₘ(° C.) |
| I/C | 50.9 | G/C | 52.8 | | |
| I/A | 47.0 | T/A | 52.8 | U/A | 51.0 |
| I/G | 43.8 | C/G | 52.8 | | |
| I/T | 43.4 | A/T | 52.8 | A/U | 51.0 |
| I/U | 39.7 | A/U | 51.0 | | |

The following rules, derived from Kawase et al. are applicable to the design and selection of dsRNA analogs according to the present disclosure. For example, dsRNA further comprising a universal-binding nucleotide that is inosine: (a) when XY=IC, Tₘ (A₂₆₀=0.5) is measured to be 51.1° C. while the corresponding wild type double-strand dsRNA melts at 59.2° C., an approximately 40 decrease per substitution in the melting temperature; (b) when XY=IA, Tₘ (A₂₆₀=0.5) is measured to be 44.7° C., while the corresponding wild type double-strand dsRNA melts at 42.3° C. (that is, replacement of two Ts with Di in the self-complementary duplex shown in Table 2B stabilizes the duplex marginally—about 1.2° C. per substitution); (c) when XY=IG, Tₘ (A₂₆₀=0.5) is measured to be only 35.0° C. while the corresponding wild type double-strand dsRNA (XY=CG) melts at 51.0° C., an approximately 8° C. decrease per substitution in the melting temperature; (d) when XY=IT, the dsRNA duplex is not expected to show cooperative melting, but the wild sequence (XY=AT) melts at 54.8° C. (indicating that the I-T nucleotide pair is very unstable—that is, replacement of 2 As in the dsRNA duplex with two dls; (e) incorporation of 4 Di in the duplex presented in Table 2B destabilizes the duplex significantly.

From the thermodynamic values calculated using van't Hoff plots according to a two state model, Kawase et al., conclude that the sequence of purine-pyrimidine is favored in double strand formation due to nucleotide stacking. For instance the duplex formation of XY=AT is more favored formation than an XY=CG and TA (see Table 2B).

TABLE 2B
Tₘ Values of Self-complementary Duplexes

| d(GGGAAXYTTCCC) | Tₘ | Tₘ | Tₘ | Tₘ | Tₘ |
|---|---|---|---|---|---|
| (SEQ ID NO: 222) | (A₂₆₀ = 0.25) | (A₂₆₀ = 0.5) | (A₂₆₀ = 1.0) | (A₂₆₀ = 2.0) | (A₂₆₀ = 3.0) |
| IC | 48.5 | 51.1 | 52.6 | 55.0 | 55.8 |
| IA | 42.5 | 44.7 | 45.8 | 48 | 49.0 |
| IG | — | 35.0 | 36.5 | 38.3 | 39.7 |
| IT | — | — | — | — | — |
| II | — | — | — | — | — |
| GC | 56.5 | 59.2 | 60.7 | 62.8 | 63.5 |
| GA | 42.0 | 44.1 | 45.9 | 48.5 | 50.3 |
| GG | — | 33.2 | 36.7 | 38.4 | 40.8 |
| GT | — | — | — | — | — |
| AT | 51.6 | 54.8 | 57.0 | 58.0 | 58.8 |
| TA | 40.6 | 42.3 | 43.9 | 45.2 | 45.9 |
| CG | 50.4 | 51.0 | 52.2 | 55.5 | 56.2 |
| AC | — | — | — | — | — |
| CT | — | — | — | — | — |

Note 1:
Tₘs were measured at various concentrations and have been shown by their A₂₆₀.
Note 2:
Where there is no date, the duplex did not show cooperative melting.

As a person of skill in the art would understand, although universal-binding nucleotides are used herein as an example of determining stability (i.e., the Tₘ value), other nucleotide substitutions (e.g., 5-methyluridine for uridine and the complementary 2,6-diaminopurine for adenine) or further modifications (e.g., a ribose modification at the 2′-position) can also be evaluated by these or similar methods.

Alternative embodiments provide methods for selecting one or more dsRNA or analog thereof further comprising a universal-binding nucleotide, which methods employ “off-target” profiling whereby one or more dsRNA further comprising a universal-binding nucleotide is contacted with a cell, either in vivo or in vitro, and total target mRNA is collected, and used to probe a microarray comprising oligonucleotides having one or more nucleotide sequence from a panel of known genes, including non-target genes. The “off-target” profile of the dsRNA analog is quantified by determining the number of non-target genes having reduced expression levels in the presence of a dsRNA further comprising a universal-binding nucleotide. The existence of “off-target” binding indicates a dsRNA containing a universal-binding nucleotide is capable of binding one or more non-target gene messages. In certain embodiments, a dsRNA or analog thereof further comprising a universal-binding nucleotide applicable to therapeutic use will exhibit a high Tₘ value while exhibiting little or no “off-target” binding.

Within other aspects of the present disclosure there are provided methods that employ one or more dsRNA or analogs thereof, and compositions comprising one or more dsRNA, wherein at least one of the dsRNA further comprise one or more universal-binding nucleotide in the first, second or third position in the anti-codon of the antisense strand of a dsRNA and is capable of specifically binding to a target RNA (e.g., in a human cell or subject).

Within certain embodiments, methods disclosed herein comprise the steps of (a) designing or synthesizing a suitable dsRNA for RNAi gene silencing of a target gene, wherein the dsRNA comprises at least one base pair comprising a 5-methyluridine base paired with a 2,6-diaminopurine and further comprises one or more universal-binding nucleotide in the antisense strand; and (b) contacting a cell expressing target with the dsRNA, wherein the dsRNA is capable of specifically binding to a target mRNA or gene, thereby reducing the target's expression level.

In further embodiments, methods are provided wherein one or more anti-codon within the antisense strand of a dsRNA molecule or analog thereof is substituted in a first position (i.e., the wobble nucleotide position) in an anti-codon of the antisense strand with a universal-binding nucleotide. Without wishing to be bound by theory but relying on the wobble hypothesis, the first nucleotide-pair substitution allows the antisense strand of a substituted dsRNA molecule to specifically bind to a target RNA wherein a first nucleotide pair substitution has occurred, but which substitution does not result in an amino acid change in the corresponding target gene product owing to the redundancy of the genetic code.

In still further embodiments of the presently disclosed methods, one or more anti-codon within an antisense strand of a dsRNA molecule or analog thereof is substituted with a universal-binding nucleotide in a second or third position in the anti-codon of the antisense strand. By substituting a universal-binding nucleotide for a first or second position, the one or more first or second position nucleotide-pair substitution allows the substituted dsRNA molecule to specifically bind to mRNA wherein a first or a second position nucleotide-pair substitution has occurred, wherein the one or more nucleotide pair substitution results in an amino acid change in the corresponding gene product.

Any of these methods of identifying dsRNA of interest can also be used to examine a dsRNA that decreases expression of a target gene by RNA interference, comprising a first strand that is complementary to the target gene mRNA and a second strand that is complementary to the first strand, wherein the first and second strands form a double-stranded region of about 10 to about 40 base pairs; wherein the double-stranded region has at least one base pair comprising a 5-methyluridine base paired with a 2,6-diaminopurine.

### Compositions and Methods of Use

In certain embodiments, the dsRNA will be specific for a target gene that is expressed at an elevated level or continues to be expressed when it should not and is a causal or contributing factor associated with, for example, an hyperproliferative, angiogenic, metabolic syndrome, cardiac, neural, inflammatory, or autoimmune disease, state, or adverse condition. In certain embodiments, the dsRNA will be specific for a target gene whose gene product regulates and/or modulates the expression of another gene that is expressed at an elevated level or continues to be expressed when it should not and is a causal or contributing factor associated with, for example, an hyperproliferative, angiogenic, metabolic syndrome, cardiac, neural, inflammatory, or autoimmune disease, state, or adverse condition. In this context, a dsRNA or analog thereof of this disclosure will effectively downregulate expression of a target gene to levels that prevent, alleviate, or reduce the severity or recurrence of one or more associated disease symptoms. Alternatively, for various distinct disease models in which expression of a target gene is not necessarily elevated as a consequence or sequel of disease or other adverse condition, down regulation of a target gene will nonetheless result in a therapeutic result by lowering gene expression (i.e., to reduce levels of a selected mRNA or protein product of a target gene). Alternatively, dsRNAs of this disclosure may specifically lower expression of a target gene, which can result in upregulation of a “downstream” gene whose expression is negatively regulated by the target protein, directly or indirectly.

Aqueous suspensions contain the active material (dsRNA) in admixture with excipients suitable for the manufacture of aqueous suspensions. Such excipients are suspending agents, for example sodium carboxymethylcellulose, methylcellulose, hydropropyl-methylcellulose, hydroxypropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

Syrups and elixirs can be formulated with sweetening agents, for example glycerol, propylene glycol, sorbitol, glucose or sucrose. Such formulations can also contain a demulcent, a preservative and flavoring, and coloring agents. The pharmaceutical compositions can be in the form of a sterile injectable aqueous or oleaginous suspension. This suspension can be formulated according to the known art using those suitable dispersing or wetting agents and suspending agents that have been mentioned above. The sterile injectable preparation can also be a sterile injectable solution or suspension in a non-toxic parentally acceptable diluent or solvent, for example as a solution in 1,3-butanediol. Among the acceptable vehicles and solvents that can be employed are water, Ringer's solution and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid find use in the preparation of injectables.

The present disclosure also includes dsRNA compositions prepared for storage or for administration that include a pharmaceutically effective amount of the desired compounds in a pharmaceutically acceptable carrier or diluent and may be sterile, non-pyrogen free. Acceptable carriers or diluents for therapeutic use are well known in the pharmaceutical art, and are described, for example, in Remington's Pharmaceutical Sciences, Mack Publishing Co., A. R. Gennaro edit., 1985, hereby incorporated by reference herein. For example, preservatives, stabilizers, dyes and flavoring agents can be provided. These include sodium benzoate, sorbic acid and esters of p-hydroxybenzoic acid. In addition, antioxidants and suspending agents can be used.

In accordance with this disclosure herein, the present disclosure provides dsRNA compositions and methods for inhibiting expression of a target gene in a cell or organism. In related embodiments, this disclosure provides methods and dsRNA compositions for treating a subject, including a human cell, tissue or individual, having a disease or at risk of developing a disease caused by the expression of a target gene. In one embodiment, the method includes administering a dsRNA of this disclosure or a pharmaceutical composition containing the dsRNA to a cell or an organism, such as a mammal, such that expression of the target gene is silenced. Mammalian subjects amendable for treatment using the compositions and methods of the present disclosure include those suffering from one or more disorders caused by target overexpression, or which are amenable to treatment by reducing expression of a target protein, including hyperproliferative, angiogenic, cardiac, neural, metabolic syndrome, inflammatory, or immune disorders. Exemplary diseases or disorders amenable to treatment using dsRNAs of this disclosure include autoimmune diseases (e.g., diabetes mellitus, rheumatoid arthritis, spondylarthritis, ankylosing spondylitis, multiple sclerosis, encephalomyelitis, inflammatory bowel disease, Chron's disease, psoriasis or psoriatic arthritis, myasthenia gravis, systemic lupus erythematosis, graft-versus-host disease, and allergies).

The dsRNA compositions of the instant disclosure can be effectively employed as pharmaceutically acceptable formulations. Pharmaceutically-acceptable formulations prevent, alter the occurrence or severity of, or treat (alleviate one or more symptom(s) to a detectable or measurable extent) of a disease state or other adverse condition in a patient. A pharmaceutically acceptable formulation includes salts of the above compounds, e.g., acid addition salts such as salts of hydrochloric acid, hydrobromic acid, acetic acid, and benzene sulfonic acid. A pharmaceutical composition or formulation refers to a composition or formulation in a form suitable for administration, e.g., systemic administration, into a cell or patient such as a human. Suitable forms, in part, depend upon the use or the route of entry, for example oral, transdermal, or by injection. Such forms should not prevent the composition or formulation from reaching a target cell (i.e., a cell to which the negatively charged nucleic acid is desirable for delivery). For example, pharmaceutical compositions injected into the blood stream should be soluble. Other factors are known in the art, and include considerations such as toxicity and forms that prevent the composition or formulation from exerting its effect.

Pharmaceutical compositions of this disclosure can also be in the form of oil-in-water emulsions. The oily phase can be a vegetable oil or a mineral oil or mixtures of these. Suitable emulsifying agents can be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions can also contain sweetening and flavoring agents.

Within certain embodiments of this disclosure, pharmaceutical compositions and methods are provided that feature the presence or administration of one or more dsRNA or analogs thereof of this disclosure, combined, complexed, or conjugated with a polypeptide, optionally formulated with a pharmaceutically-acceptable carrier, such as a diluent, stabilizer, buffer, or the like. The negatively charged dsRNA molecules of this disclosure may be administered to a patient by any standard means, with or without stabilizers, buffers, or the like, to form a composition suitable for treatment. When it is desired to use a liposome delivery mechanism, standard protocols for formation of liposomes can be followed. The compositions of the present disclosure may also be formulated and used as a tablet, capsule or elixir for oral administration, suppository for rectal administration, sterile solution, or suspension for injectable administration, either with or without other compounds known in the art. Thus dsRNAs of the present disclosure may be administered in any form, for example transdermally or by local injection.

These and other subjects are effectively treated, prophylactically or therapeutically, by administering to the subject an effective amount of one or more dsRNA(s) of this disclosure containing. Within additional aspects of this disclosure, combinatorial formulations and methods are provided comprising an effective amount of one or more dsRNA(s) of the present disclosure in combination with one or more secondary or adjunctive active agents that are combinatorially formulated or coordinately administered with the dsRNAs of this disclosure to control a target gene-associated disease or condition as described herein. Useful adjunctive therapeutic agents in these combinatorial formulations and coordinate treatment methods include, for example, enzymatic nucleic acid molecules, allosteric nucleic acid molecules, antisense, decoy, or aptamer nucleic acid molecules, antibodies such as monoclonal antibodies, small molecules and other organic or inorganic compounds including metals, salts and ions, and other drugs and active agents indicated for treating a target gene-associated disease or condition. For example, if the target is TNF, adjunctive therapies can include non-steroidal anti-inflammatory drugs (NSAIDs), methotrexate, disease-modifying antirheumatic drugs (DMARDs), or the like. The use of multiple compounds to treat an indication may increase the beneficial effects while reducing the presence of side effects.

To practice the coordinate administration methods of this disclosure, a dsRNA is administered, simultaneously or sequentially, in a coordinate treatment protocol with one or more of the secondary or adjunctive therapeutic agents contemplated herein. The coordinate administration may be done in either order, and there may be a time period while only one or both (or all) active therapeutic agents, individually or collectively, exert their biological activities. A distinguishing aspect of all such coordinate treatment methods is that the dsRNA present in the composition elicits some favorable clinical response, which may or may not be in conjunction with a secondary clinical response provided by the secondary therapeutic agent. Often, the coordinate administration of the dsRNA with a secondary therapeutic agent as contemplated herein will yield an enhanced therapeutic response beyond the therapeutic response elicited by either or both the purified dsRNA or secondary therapeutic agent alone.

In another embodiment, a dsRNA of this disclosure can include a conjugate member on one or more of the terminal nucleotides of a dsRNA. The conjugate member can be, for example, a lipophile, a terpene, a protein binding agent, a vitamin, a carbohydrate, or a peptide. For example, the conjugate member can be naproxen, nitroindole (or another conjugate that contributes to stacking interactions), folate, ibuprofen, or a C5 pyrimidine linker. In other embodiments, the conjugate member is a glyceride lipid conjugate (e.g., a dialkyl glyceride derivatives), vitamin E conjugates, or thio-cholesterols. Additional conjugate members include peptides that function, when conjugated to a modified dsRNA of this disclosure, to facilitates delivery of the dsRNA into a target cell, or otherwise enhance delivery, stability, or activity of the dsRNA when contacted with a biological sample (e.g., a target cell expressing TNF). Exemplary peptide conjugate members for use within these aspects of this disclosure, including, but not limited to peptides PN27, PN28, PN29, PN58, PN61, PN73, PN158, PN159, PN173, PN182, PN202, PN204, PN250, PN361, PN365, PN404, PN453, and PN509, are described, for example, in U.S. Patent Application Publication Nos. 2006/0040882 and 2006/0014289, which are incorporated herein by reference. In certain embodiments, when peptide conjugate partners are used to enhance delivery of dsRNA or analogs thereof of this disclosure, the resulting dsRNA formulations and methods will often exhibit further reduction of an interferon response in target cells as compared to dsRNAs delivered in combination with alternate delivery vehicles, such as lipid delivery vehicles (e.g., LIPOFECTAMINE).

In still another embodiment, a dsRNA or analog thereof of this disclosure may be conjugated to the polypeptide and admixed with one or more non-cationic lipids or a combination of a non-cationic lipid and a cationic lipid to form a composition that enhances intracellular delivery of the dsRNA as compared to delivery resulting from contacting the target cells with a naked dsRNA. In more detailed aspects of this disclosure, the mixture, complex or conjugate comprising a dsRNA and a polypeptide can be optionally combined with (e.g., admixed or complexed with) a cationic lipid, such as LIPOFECTIN. To produce these compositions comprised of a polypeptide, dsRNA and a cationic lipid, the dsRNA and peptide may be mixed together first in a suitable medium such as a cell culture medium, after which the cationic lipid is added to the mixture to form a dsRNA/delivery peptide/cationic lipid composition. Optionally, the peptide and cationic lipid can be mixed together first in a suitable medium such as a cell culture medium, followed by the addition of the dsRNA to form the dsRNA/delivery peptide/cationic lipid composition.

This disclosure also features the use of dsRNA compositions comprising surface-modified liposomes containing poly(ethylene glycol) lipids (PEG-modified, or long-circulating liposomes or stealth liposomes). These formulations offer a method for increasing the accumulation of drugs in target tissues. This class of drug carriers resists opsonization and elimination by the mononuclear phagocytic system (MPS or RES), thereby enabling longer blood circulation times and enhanced tissue exposure for the encapsulated drug (Lasic et al., Chem. Rev. 95:2601-2627, 1995; Ishiwata et al., Chem. Pharm. Bull. 43:1005-1011, 1995). Such liposomes have been shown to accumulate selectively in tumors, presumably by extravasation and capture in the neovascularized target tissues (Lasic et al., Science 267:1275-1276, 1995; Oku et al., Biochim. Biophys. Acta 1238:86-90, 1995). The long-circulating liposomes enhance the pharmacokinetics and pharmacodynamics of DNA and RNA, particularly compared to conventional cationic liposomes which are known to accumulate in tissues of the MPS (Liu et al., J. Biol. Chem. 42:24864-24870, 1995; Choi et al., PCT Publication No. WO 96/10391; Ansell et al., PCT Publication No. WO 96/10390; Holland et al., PCT Publication No. WO 96/10392). Long-circulating liposomes are also likely to protect drugs from nuclease degradation to a greater extent compared to cationic liposomes, based on their ability to avoid accumulation in metabolically aggressive MPS tissues such as the liver and spleen.

In one embodiment, this disclosure provides compositions suitable for administering dsRNA molecules of this disclosure to specific cell types, such as hepatocytes. For example, the asialoglycoprotein receptor (ASGPr) (Wu and Wu, J. Biol. Chem. 262:4429-4432, 1987) is unique to hepatocytes and binds branched galactose-terminal glycoproteins, such as asialoorosomucoid (ASOR). Binding of such glycoproteins or synthetic glycoconjugates to the receptor takes place with an affinity that strongly depends on the degree of branching of the oligosaccharide chain, for example, triatennary structures are bound with greater affinity than biatenarry or monoatennary chains (Baenziger and Fiete, Cell 22: 611-620, 1980; Connolly et al., J. Biol. Chem. 257:939-945, 1982). Lee and Lee (Glycoconjugate J. 4:317-328, 1987) obtained this high specificity through the use of N-acetyl-D-galactosamine as the carbohydrate moiety, which has higher affinity for the receptor compared to galactose. This “clustering effect” has also been described for the binding and uptake of mannosyl-terminating glycoproteins or glycoconjugates (Ponpipom et al., J. Med. Chem. 24:1388-1395, 1981). The use of galactose and galactosamine based conjugates to transport exogenous compounds across cell membranes can provide a targeted delivery approach to the treatment of liver disease. The use of bioconjugates can also provide a reduction in the required dose of therapeutic compounds required for treatment. Furthermore, therapeutic bioavailability, pharmacodynamics, and pharmacokinetic parameters can be modulated through the use of dsRNA bioconjugates of this disclosure.

The use of a liposome or other drug carrier comprising dsRNA of the instant disclosure can potentially localize the drug, for example, in certain tissue types, such as the tissues of the reticular endothelial system (RES). A liposome formulation that can facilitate the association of drug with the surface of certain cells or tissues, such as neural cells, cardiac cells, liver cells, pancreatic cells, lymphocytes, or macrophages, which may be useful for targeting genes that are predominantly expressed in such cells.

One embodiment of the present disclosure provides nanoparticles less than 100 nanometers (nm) comprising dsRNA that decreases expression of a target gene by RNAi. More specifically, the dsRNA is less than about 30 base pairs in length, or is from about 20 to about 25 base pairs in length.

The present disclosure also features a method for preparing dsRNA nanoparticles. A first solution containing melamine derivatives is dissolved in an organic solvent such as dimethyl sulfoxide, or dimethyl formamide to which an acid such as HCl has been added. The concentration of HCl would be about 3.3 moles of HCl for every mole of the melamine derivative. The first solution is then mixed with a second solution, which includes a nucleic acid dissolved or suspended in a polar or hydrophilic solvent (e.g., an aqueous buffer solution containing, for instance, ethylenediaminetetraacetic acid (EDTA), or tris(hydroxymethyl) aminomethane (TRIS), or combinations thereof. The mixture forms a first emulsion. The mixing can be done using any standard technique such as, for example sonication, vortexing, or in a microfluidizer. This causes complexing of the nucleic acids with the melamine derivative forming a trimeric nucleic acid complex. While not being bound to theory or mechanism, it is believed that three nucleic acids are complexed in a circular fashion about one melamine derivative moiety, and that a number of the melamine derivative moieties can be complexed with the three nucleic acid molecules depending on the size of the number of nucleotides that the nucleic acid has. The concentration should be from about 1 to about 7 moles of the melamine derivative for every mole of a double-stranded nucleic acid having about 20 nucleotide pairs, more if the double-stranded nucleic acid is larger. The resultant nucleic acid particles can be purified and the organic solvent removed using size-exclusion chromatography or dialysis or both.

The complexed nucleic acid nanoparticles can then be mixed with an aqueous solution containing either polyarginine or a Gln-Asn polymer, or both, in an aqueous solution. A preferred molecular weight of each polymer is about 5000 to about 15,000 Daltons. This forms a solution containing nanoparticles of nucleic acid complexed with the melamine derivative and the polyarginine and the Gln-Asn polymers. The mixing steps are carried out in a manner that minimizes shearing of the nucleic acid while producing nanoparticles on average smaller than about 200 nanometers in diameter. While not wishing to be bound by theory, it is believed that the polyarginine complexes with the negative charge of the phosphate groups within the minor groove of the nucleic acid, and the polyarginine wraps around the trimeric nucleic acid complex. At either terminus of the polyarginine other moieties, such as the TAT polypeptide, mannose or galactose, can be covalently bound to the polymer to direct binding of the nucleic acid complex to specific tissues, such as to the liver when galactose is used. While not being bound to theory, it is believed that the Gln-Asn polymer complexes with the nucleic acid complex within the major groove of the nucleic acid through hydrogen bonding with the bases of the nucleic acid. The polyarginine and the Gln-Asn polymer should be present at a concentration of 2 moles per every mole of nucleic acid having 20 base pairs. The concentration should be increased proportionally for a nucleic acid having more than 20 base pairs. So perhaps, if the nucleic acid has 25 base pairs, the concentration of the polymers should be 2.5-3 moles per mole of double-stranded nucleic acid. An example of is a polypeptide operatively linked to an N-terminal protein transduction domain from HIV TAT. The HIV TAT construct for use in such a protein is described in detail in Vocero-Akbani et al., Nature Med. 5:23-33, 1999. See also, U.S. Patent Application Publication No. 2004/0132161, published on Jul. 8, 2004. The resultant nanoparticles can be purified by standard means such as size exclusion chromatography followed by dialysis. The purified complexed nanoparticles can then be lyophilized using techniques well known in the art.

A pharmaceutically effective dose is that dose required to prevent, inhibit the occurrence, or treat (alleviate a symptom to some extent, preferably all of the symptoms) of a disease state. The pharmaceutically effective dose depends on the type of disease, the composition used, the route of administration, the type of mammal being treated, the physical characteristics of the specific mammal under consideration, concurrent medication, and other factors that those skilled in the medical arts will recognize. For example, an amount between 0.1 mg/kg and 100 mg/kg body weight/day of active ingredients is administered dependent upon potency of the dsRNAs of this disclosure.

Dosage levels of the order of from about 0.1 mg to about 140 mg per kilogram of body weight per day are useful in the treatment of the above-indicated conditions (about 0.5 mg to about 7 g per patient per day). The amount of active ingredient that can be combined with the carrier materials to produce a single dosage form varies depending upon the host treated and the particular mode of administration. Dosage unit forms generally contain between from about 1 mg to about 500 mg of an active ingredient.

It is understood that the specific dose level for any particular patient depends upon a variety of factors including the activity of the specific compound employed, the age, body weight, general health, sex, diet, time of administration, route of administration, and rate of excretion, drug combination and the severity of the particular disease undergoing therapy. Following administration of dsRNA compositions according to the formulations and methods of this disclosure, test subjects will exhibit about a 10% up to about a 99% reduction in one or more symptoms associated with the disease or disorder being treated, as compared to placebo-treated or other suitable control subjects.

Nucleic acid molecules and polypeptides can be administered to cells by a variety of methods known to those of skill in the art, including administration within formulations that comprise the dsRNA and polypeptide alone, or that further comprise one or more additional components, such as a pharmaceutically acceptable carrier, diluent, excipient, adjuvant, emulsifier, buffer, stabilizer, preservative, or the like. In certain embodiments, the dsRNA or the polypeptide can be encapsulated in liposomes, administered by iontophoresis, or incorporated into other vehicles, such as hydrogels, cyclodextrins, biodegradable nanocapsules, bioadhesive microspheres, or proteinaceous vectors (see, e.g., PCT Publication No. WO 00/53722). Alternatively, a nucleic acid/peptide/vehicle combination can be locally delivered by direct injection or by use of an infusion pump. Direct injection of the nucleic acid molecules of this disclosure, whether subcutaneous, intramuscular, or intradermal, can take place using standard needle and syringe methodologies, or by needle-free technologies, such as those described in Conry et al., Clin. Cancer Res. 5:2330-2337, 1999, and PCT Publication No. WO 99/31262.

The dsRNAs can also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

For administration to non-human animals, the composition can also be added to the animal feed or drinking water. It can be convenient to formulate the animal feed and drinking water compositions so that the animal takes in a therapeutically appropriate quantity of the composition along with its diet. It can also be convenient to present the composition as a premix for addition to the feed or drinking water.

Further methods for delivery of nucleic acid molecules, such as the dsRNAs of this disclosure, are described, for example, in Boado et al., J. Pharm. Sci. 87:1308-1315, 1998; Tyler et al., FEBS Lett. 421:280-284, 1999; Pardridge et al., Proc. Nat'l Acad. Sci. USA 92:5592-5596, 1995; Boado, Adv. Drug Delivery Rev. 15:73-107, 1995; Aldrian-Herrada et al., Nucleic Acids Res. 26:4910-4916, 1998; Tyler et al., Proc. Nat'l Acad. Sci. USA 96:7053-7058, 1999; Akhtar et al., Trends Cell Bio. 2:139, 1992; “Delivery Strategies for Antisense Oligonucleotide Therapeutics,” ed. Akhtar, 1995, Maurer et al., Mol. Membr. Biol. 16:129-140, 1999; Hofland and Huang, Handb. Exp. Pharmacol 137:165-192, 1999; and Lee et al., ACS Symp. Ser. 752:184-192, 2000. Sullivan et al., PCT Publication No. WO 94/02595, further describe general methods for delivery of enzymatic nucleic acid molecules. These protocols can be utilized to supplement or complement delivery of virtually any dsRNA contemplated within this disclosure.

In addition to in vivo gene inhibition, a skilled artisan will appreciate that the dsRNA and analogs thereof of the present disclosure are useful in a wide variety of in vitro applications. Such in vitro applications, include, for example, scientific and commercial research (e.g., elucidation of physiological pathways, drug discovery and development), and medical and veterinary diagnostics. In general, the method involves the introduction of the dsRNA agent into a cell using known techniques (e.g., absorption through cellular processes, or by auxiliary agents or devices, such as electroporation, lipofection, or through the use of peptide conjugates), then maintaining the cell for a time sufficient to obtain degradation of a target mRNA.

All U.S. patents, U.S. patent application publications, U.S. patent applications, foreign patents, foreign patent applications, non-patent publications, figures, tables, and websites referred to in this specification are expressly incorporated herein by reference, in their entirety.

### Examples

The above disclosure generally describes the present disclosure, which is further exemplified by the following examples. These specific examples are described solely for purposes of illustration, and are not intended to limit the scope of this disclosure. Although specific targets, terms, and values have been employed herein, such targets, terms, and values will likewise be understood as exemplary and non-limiting to the scope of this disclosure.

### Example 1

### Melting Temperature of drtRNA Duplexes

The melting temperature (Tm) of dsRNA having one or more 5-methyluridine:2,6-diaminopurine base pairs (drtRNA) is examined.

The thermal melting profiles of the dsRNA are recorded on a Shimadzu UV-VIS 1601 with thermoelectrically temperature controlled through the Peltier device. The temperature is changed at a rate of 0.5° C./minute from 90° C. to 20° C. while absorption is recorded at 260 nm. Additionally, a melting temperature profile is done whereby the temperature is changed at a rate of 0.5° C./minute from 20° C. to 90° C.

In this experiment, curves are analyzed and Tₘs are extracted using a “differential curves” method where the “inflection point” of the equilibrium melting curves is determine from the maximum of the derivative curve.

The nucleic acid sequence of the sense and antisense strands (both shown in the 5′ to 3′ orientation) of the dsRNA are shown below.

### MAPK14 Nucleotide Sequence of the Sense and Antisense Strands

| | |
|---|---|
| Sense Strand | |
| 5′- CCUACAGAGAACUGCGGUUTT - 3′ | (SEQ ID NO: 223) |
| Antisense Strand | |
| 5′- AACCGCAGUUCUCUGUAGGTT - 3′ | (SEQ ID NO: 224) |

The MAPK14 dsRNA comprises a 21 nucleotide antisense strand and a 21 nucleotide sense strand, which anneal to form a double-stranded region of 19 base pairs with two 3′ end overhangs. The MPAK14 dsRNA is used as a template for a dsRNA comprising 5-methyluridine:2,6-diaminopurine base pairs. Nicked dsRNAs comprising 5-methyluridine:2,6-diaminopurine base pairs are also are tested. Table 3 below shows the sense and antisense strands of various MAPK14 dsRNAs comprising 5-methyluridine(s) and 2,6-diaminopurines. A 5-methyluridine within a dsRNA is indicated by a “‘t”; a 2,6-diaminopurine within a dsRNA is indicated by a “A^{2/6}” (applies to all sequences in this Example). A nick in a nucleotide sequence is indicated by a single parentheses (i.e., ‘). For example, the MAPK14.NkdS10 dsRNA has a nick in the sense strand between nucleotides 10 and 11, counting from the 5′ end of the sense strand (see Table 3 below).

TABLE 3

Nucleotide Sequence of Sense and Antisense Strands
of MAPK14 dsRNA

| | | | |
|---|---|---|---|
| | | | SEQ ID |
| Identifier | Strand | Nucleotide Sequence (5′ to 3′) | NO |

| | | | |
|---|---|---|---|
| MAPK14:565.17 | Sense | CCUA^{2/6}CA^{2/6}GA^{2/6}GA^{2/6}A^{2/6}CUGCGGUUTT | 225 |
| | Antisense | AACCGCAGttCtCtGtAGGTT | 226 |
| MAPK14.18 | Sense | CCtACAGAGAACtGCGGttTT | 227 |
| | Antisense | A^{2/6}A^{2/6}CCGCA^{2/6}GUUCUCUGUA^{2/6}GGTT | 228 |
| MAPK14.19 | Sense | CCtA^{2/6}CA^{2/6}GA^{2/6}GA^{2/6}A^{2/6}CtGCGGttTT | 229 |
| | Antisense | A^{2/6}A^{2/6}CCGCA^{2/6}GttCtCtGtA^{2/6}GGTT | 230 |
| MAPK14.NkdS10 | Sense | CCUACAGAGA′ACUGCGGUUTT | 231 |
| | Antisense | AACCGCAGUUCUCUGUAGGTT | 232 |
| MAPK14.NkdS10.5 | Sense | CCUA^{2/6}CA^{2/6}GA^{2/6}GA^{2/6}′A^{2/6}CUGCGGUUTT | 233 |
| | Antisense | AACCGCAGUUCUCUGUAGGTT | 234 |
| MAPK14.NkdS10.6 | Sense | CCUA^{2/6}CA^{2/6}GA^{2/6}GA^{2/6}′A^{2/6}CUGCGGUUTT | 235 |
| | Antisense | AACCGCAGttCtCtGtAGGTT | 236 |
| MAPK14.NkdS10.7 | Sense | CCtACAGAGA′ACtGCGGttTT | 237 |
| | Antisense | AACCGCAGUUCUCUGUAGGTT | 238 |
| MAPK14.NkdS10.8 | Sense | CCtACAGAGA′ACtGCGGttTT | 239 |
| | Antisense | A^{2/6}A^{2/6}CCGCA^{2/6}GUUCUCUGUA^{2/6}GGTT | 240 |
| MAPK14.NkdS10.9 | Sense | CCtA^{2/6}CA^{2/6}GA^{2/6}GA^{2/6}′A^{2/6}CtGCGGttTT | 241 |
| | Antisense | A^{2/6}A^{2/6}CCGCA^{2/6}GttCtCtGtA^{2/6}GGTT | 242 |

### SOS1 Nucleotide Sequence of the Sense and Antisense Strands

| | |
|---|---|
| Sense Strand | |
| 5′- AUUGACCACCAGGUUUCUGTT - 3′ | (SEQ ID NO: 243) |
| Antisense Strand | |
| 5′- CAGAAACCUGGUGGUCAAUTT - 3′ | (SEQ ID NO: 244) |

The SOS1 dsRNA comprises a 21 nucleotide antisense strand and a 21 nucleotide sense strand, which anneal to form a double-stranded region of 19 base pairs with two 3′ end overhangs. The SOS1 dsRNA is used as a template for a dsRNA comprising 5-methyluridine:2,6-diaminopurine base pairs. Nicked dsRNAs comprising 5-methyluridine:2,6-diaminopurine base pairs also are tested. Table 4 below shows the sense and antisense strands of various SOS1 dsRNAs comprising 5-methyluridine(s) and 2,6-diaminopurines. A locked nucleic acid (LNA) is indicated by an underline (e.g., A is an adenine comprising an LNA, T is a thymine comprising an LNA, G is a guanine comprising an LNA, etc. . . . ). A nick in a nucleotide sequence is indicated by a single parentheses (i.e., ‘). For example, the SOS1Nkd8.2 dsRNA has a nick in the sense strand between nucleotides 8 and 9, counting from the 5′ end of the sense strand (see Table 4 below).

TABLE 4

Nucleotide Sequence of Sense and Antisense Strands
of SOS1 dsRNA

| | | | SEQ ID |
|---|---|---|---|
| Identifier | Strand | Nucleotide Sequence (5′ to 3′) | NO |
| SOS1:364.17 | Sense | A^{2/6}UUGA^{2/6}CCA^{2/6}CCA^{2/6}GGUUUCUGTT | 245 |
| | Antisense | CAGAAACCtGGtGGtCAAtTT | 246 |
| SOS1:364.18 | Sense | AttGACCACCAGGtttCtGTT | 247 |
| | Antisense | CA^{2/6}GA^{2/6}A^{2/6}A^{2/6}CCUGGUGGUCA^{2/6}A^{2/6}UTT | 248 |
| SOS1:364.19 | Sense | A^{2/6}ttGA^{2/6}CCA^{2/6}CCA^{2/6}GGtttCtGTT | 249 |
| | Antisense | CA^{2/6}GA^{2/6}A^{2/6}A^{2/6}CCtGGtGGtCA^{2/6}A^{2/6}tTT | 250 |
| SOS1:364Nkd8.2 | Sense | ATUGACCA′CCAGGUUTCUGTT | 251 |
| | Antisense | CAGAAACCUGGUGGUCAAUTT | 252 |
| SOS1:364(S-4ln) | Sense | ATUGACCACCAGGUUTCUGTT | 253 |
| | Antisense | CAGAAACCUGGUGGUCAAUTT | 254 |
| SOS1:364Nkd8.3 | Sense | AUUGACCA′CCAGGUUTCUGTT | 255 |
| | Antisense | CAGAAACCUGGUGGUCAAUTT | 256 |
| SOS1:364Nkd8.5 | Sense | A^{2/6}UUGA^{2/6}CCA^{2/6}′CCA^{2/6}GGUUUCUGTT | 257 |
| | Antisense | CAGAAACCUGGUGGUCAAUTT | 258 |
| SOS1:364Nkd8.6 | Sense | A^{2/6}UUGA^{2/6}CCA^{2/6}′CCA^{2/6}GGUUUCUGTT | 259 |
| | Antisense | CAGAAACCtGGtGGtCAAtTT | 260 |
| SOS1:364Nkd8.7 | Sense | AttGACCA′CCAGGtttCtGTT | 261 |
| | Antisense | CAGAAACCUGGUGGUCAAUTT | 262 |
| SOS1:364Nkd8.8 | Sense | AttGACCA′CCAGGtttCtGTT | 263 |
| | Antisense | CA^{2/6}GA^{2/6}A^{2/6}A^{2/6}CCUGGUGGUCA^{2/6}A^{2/6}UTT | 264 |
| SOS1:364Nkd8.9 | Sense | A^{2/6}ttGA^{2/6}CCA^{2/6}′CCA^{2/6}GGtttCtGTT | 265 |
| | Antisense | CA^{2/6}GA^{2/6}A^{2/6}A^{2/6}CCtGGtGGtCA^{2/6}A^{2/6}tTT | 266 |

### ApoB:2100 Nucleotide Sequence of the Sense and Antisense Strands

| | |
|---|---|
| Sense Strand | |
| 5′- AAUCUUAUAUUUGAUCCAATT - 3′ | (SEQ ID NO: 267) |
| Antisense Strand | |
| 5′- UUGGAUCAAAUAUAAGAUUTT - 3′ | (SEQ ID NO: 268) |

The ApoB:2100 dsRNA comprises a 21 nucleotide antisense strand and a 21 nucleotide sense strand, which anneal to form a double-stranded region of 19 base pairs with two 3′ end overhangs. The ApoB:2100 dsRNA is used as a template for a dsRNA comprising 5-methyluridine:2,6-diaminopurine base pairs. Nicked dsRNAs comprising 5-methyluridine:2,6-diaminopurine base pairs also are tested. Table 5 below shows the sense and antisense strands of various ApoB:2100 dsRNAs comprising 5-methyluridine(s) and 2,6-diaminopurines. A locked nucleic acid (LNA) is indicated by an underline (e.g., A is an adenine comprising an LNA, T is a thymine comprising an LNA, G is a guanine comprising an LNA, etc. . . . ). A nick in a nucleotide sequence is indicated by a single parentheses (i.e., ‘). For example, theApoB:2100Nkd10 dsRNA has a nick in the sense strand between nucleotides 10 and 11, counting from the 5′ end of the sense strand (see Table 5 below).

TABLE 5

Nucleotide Sequence of Sense and Antisense Strands
of ApoB:2100 dsRNA

| | | | SEQ ID |
|---|---|---|---|
| Identifier | Strand | Nucleotide Sequence (5′ to 3′) | NO |
| ApoB:2100.17 | Sense | A^{2/6}A^{2/6}UCUUA^{2/6}UA^{2/6}UUUGA^{2/6}UCCA^{2/6}A^{2/6}TT | 269 |
| | Antisense | AttGGtAttCAGtGtGAtGTT | 270 |
| ApoB:2100.18 | Sense | CAtCACACtGAAtACCAAtTT | 271 |
| | Antisense | UUGGA^{2/6}UCA^{2/6}A^{2/6}A^{2/6}UA^{2/6}UA^{2/6}A^{2/6}GA^{2/6}UUTT | 272 |
| ApoB:2100.19 | Sense | CA^{2/6}tCA^{2/6}CA^{2/6}CtGA^{2/6}A^{2/6}tA^{2/6}CCA^{2/6}A^{2/6}tTT | 273 |
| | Antisense | ttGGA^{2/6}tCA^{2/6}A^{2/6}A^{2/6}tA^{2/6}tA^{2/6}A^{2/6}GA^{2/6}ttTT | 274 |
| ApoB:10169.1 | Sense | CAtCACACtGAAtACCAAtTT | 275 |
| | Antisense | AttGGtAttCAGtGtGAtGTT | 276 |
| ApoB:2100NkdS10 | Sense | AAUCUUAUAU′UUGAUCCAATT | 277 |
| | Antisense | UUGGAUCAAAUAUAAGAUUTT | 278 |
| ApoB:2100NkdS10.5 | Sense | A^{2/6}A^{2/6}UCUUA^{2/6}UA^{2/6}U′UUGA^{2/6}UCCA^{2/6}A^{2/6}TT | 279 |
| | Antisense | UUGGAUCAAAUAUAAGAUUT | 280 |
| ApoB:2100NkdS10.6 | Sense | A^{2/6}A^{2/6}UCUUA^{2/6}UA^{2/6}U′UUGA^{2/6}UCCA^{2/6}A^{2/6}TT | 281 |
| | Antisense | AttGGtAttCAGtGtGAtGTT | 282 |
| ApoB:2100NkdS10.7 | Sense | AAtCttAtAt′ttGAtCCAATT | 283 |
| | Antisense | UUGGAUCAAAUAUAAGAUUTT | 284 |
| ApoB:2100NkdS10.8 | Sense | AAtCttAtAt′ttGAtCCAATT | 285 |
| | Antisense | UUGGA^{2/6}UCA^{2/6}A^{2/6}A^{2/6}UA^{2/6}UA^{2/6}A^{2/6}GA^{2/6}UUTT | 286 |
| ApoB:2100NkdS10.9 | Sense | A^{2/6}A^{2/6}tCttA^{2/6}tA^{2/6}t′ttG A^{2/6}tCCA^{2/6}A^{2/6}TT | 287 |
| | Antisense | ttGGA^{2/6}tCA^{2/6}A^{2/6}A^{2/6}tA^{2/6}tA^{2/6}A^{2/6}GA^{2/6}ttTT | 288 |

### Example 2

### Melting Temperature of drtRNA ApoB Duplexes

The melting temperature (Tm) of an ApoB2100 dsRNA having one or more 5-methyluridine:2,6-diaminopurine base pairs (drtRNA) is examined.

As described above, the thermal melting profiles of the dsRNA are recorded on a Shimadzu UV-VIS 1601 with thermoelectrically temperature controlled through the Peltier device. The temperature is changed at a rate of 0.5° C./minute from 90° C. to 20° C. while absorption is recorded at 260 nm (the “Tₘ Down” data). Additionally, a melting temperature profile is done whereby the temperature is changed at a rate of 0.5° C./minute from 20° C. to 90° C. the “Tₘ Up” data).

Each ApoB dsRNA solution was prepared at 1 μM concentration by diluting the corresponding stock solution of 100 μM in water in 10 mM PBS, pH 7.2 containing 0.1 M NaCl. The Tₘ Up and Tₘ Down values for an ApoB2100 dsRNA having one, three or five 5-methyluridine nucleotides; or one, three or five 2,6-diaminopurine nucleotides; or one, three or five more 5-methyluridine:2,6-diaminopurine base pairs was obtained.

The Tₘ value for each ApoB2100 dsRNA is shown below in Table 6.

TABLE 6
Tₘ Values

| | Tₘ | Tₘ | Ave. | Tₘ Increase/ | Ave. Tₘ |
|---|---|---|---|---|---|
| Identifier | Up | Down | Tₘ | b(p) | Increase |
| ApoB:2100 unmofidied | 57.27 | 58.20 | 57.60 | 0.00 | |
| ApoB:2100 | 57.10 | 58.38 | 57.74 | 0.14 | 0.41 |
| One 5-methyluridine | | | | | |
| ApoB:2100 | 59.02 | 59.39 | 59.21 | 0.53 | |
| Three 5-methyluridines | | | | | |
| ApoB:2100 | 59.94 | 60.71 | 60.33 | 0.55 | |
| Five 5-methyluridines | | | | | |
| ApoB:2100 | 59.30 | 59.59 | 59.45 | 1.85 | 1.87 |
| One 2,6-diaminopurine | | | | | |
| ApoB:2100 | 63.45 | 63.83 | 63.64 | 2.01 | |
| Three 2,6- | | | | | |
| diaminopurines | | | | | |
| ApoB:2100 | 65.91 | 66.79 | 66.35 | 1.75 | |
| Five 2,6-diaminopurines | | | | | |
| ApoB:2100 | 60.29 | 60.37 | 60.33 | 2.73 | 2.80 |
| One 5-methyluridine:2,6- | | | | | |
| diaminopurine base pair | | | | | |
| ApoB:2100 | 66.66 | 67.75 | 67.21 | 3.20 | |
| Three 5- | | | | | |
| methyluridine:2,6- | | | | | |
| diaminopurine base pairs | | | | | |
| ApoB:2100 | 69.37 | 70.65 | 70.01 | 2.48 | |
| Five 5-methyluridine:2,6- | | | | | |
| diaminopurine base pairs | | | | | |

In general, the results in Table 6 showed that the presence of one or more 5-methyluridines or 2,6-diaminopurines increased the Tₘ of the dsRNA. Further, the results showed that the one or more 5-methyluridine:2,6-diaminopurine base pair increased the Tₘ of the dsRNA. A 5-methyluridine:2,6-diaminopurine base pair on average confers about a 2.8° C. increase in the Tₘ of the dsRNA.

### Example 3

### Nuclease Stability of drtRNA Duplexes

The nuclease stability of dsRNA having one or more 5-methyluridine:2,6-diaminopurine base pairs (drtRNA) is examined.

A 20 μg aliquot of each drtRNA duplex are mixed with 200 μl of fresh rat plasma and incubated at 37° C. At various time points (0, 30, 60 and 120 min), a 50 μl sample of the mixture are removed and immediately extracted by phenol:chloroform. The samples are dried following precipitation by adding 2.5 volumes of isopropanol alcohol and a subsequent washing step with 70% ethanol. After dissolving the dried sample in water and gel loading buffer, the samples are analyzed on a 20% polyacrylamide gel, containing 7 M urea. The degree of degradation for each sample at the various time points is visualized by ethidium bromide staining and quantitated by densitometry.

### Example 4

### Knockdown of Target Gene Expression by drtRNA Duplexes

The activity of dsRNA having one or more 5-methyluridine:2,6-diaminopurine base pairs (drtRNA) in silencing a target mRNA is compared to the dsRNA without a 5-methyluridine:2,6-diaminopurine base pair.

### Transfection and Dual Luciferase Assay

The reporter plasmid psiCHECK™-2 (Promega, Madison, Wis.), which constitutively expresses both firefly luc2 (Photinus pyralis) and Renilla (Renilla reniform is, also known as sea pansy) luciferases, was used to clone in a portion of the target gene downstream of the Renilla translational stop codon which results in a Renilla-target gene fusion mRNA. The firefly luciferase in the psiCHECK™m-2 vector was used to normalize Renilla luciferase expression and served as a control for transfection efficiency.

Multi-well plates were seeded with HeLa S3 cells/well in 100 μl Ham's F12 medium and 10% fetal bovine serum, and incubated overnight at 37° C./5% CO₂. The HeLa S3 cells were transfected with the psiCHECK™-target gene plasmid (75 ng) and a dsRNA (e.g., drtRNA, unmodified dsRNA, Qneg control dsRNA at a final concentration of 10 nM or 100 nM) formulated in Lipofectamine™ 2000 and OPTIMEM reduced serum medium. The transfection mixture was incubated with the HeLa S3 cells with gentle shaking at 37° C. for about 18 to 20 hours.

After transfecting, firefly luciferase reporter activity was measured first by adding Dual-Glo™ Luciferase Reagent (Promega, Madison, Wis.) for 10 minutes with shaking, and then quantitating the luminescent signal using a VICTOR³™ 1420 Multilabel Counter (PerkinElmer, Waltham, Mass.). After measuring the firefly luminescence, Stop & Glo® Reagent (Promega, Madison, Wis.) was added for 10 minutes with shaking to simultaneously quench the firefly reaction and initiate the Renilla luciferase reaction, and then the Renilla luciferase luminescent signal was quantitated VICTOR³™ 1420 Multilabel Counter (PerkinElmer, Waltham, Mass.).

The target gene knockdown activity for an ApoB2100 dsRNA having one, three or five 5-methyluridine nucleotides; or one, three or five 2,6-diaminopurine nucleotides; or one, three or five more 5-methyluridine:2,6-diaminopurine base pairs was compared to an unmodified ApoB2100 dsRNA and a negative control (random nucleotide sequence, “Qneg” dsRNA).

The target gene knockdown activity for each ApoB2100 dsRNA is shown below in Table 7. A smaller number indicates greater knockdown.

TABLE 7
Gene Target Knockdown Activity

| | | Relative Knockdown | |
|---|---|---|---|
| | | (Renilla/FF Ratio) | |
| | | 0.25 nM | 25 nM |
| Modification | Identifier | dsRNA | dsRNA |
| N/A | Plasmid DNA | 10.7 | 9.6 |
| None | Qneg dsRNA | 9.5 | 10.5 |
| None | ApoB:2100 unmofidied | 6.4 | 3.3 |
| 5-methyluridine | ApoB:2100 | 7 | 2.5 |
| | One 5-methyluridine | | |
| | ApoB:2100 | 4.7 | 1.5 |
| | Three 5-methyluridines | | |
| | ApoB:2100 | 5.1 | 1.7 |
| | Five 5-methyluridines | | |
| 2,6-diaminopurine | ApoB:2100 | 6.4 | 3.2 |
| | One 2,6-diaminopurine | | |
| | ApoB:2100 | 6.4 | 2.6 |
| | Three 2,6-diaminopurines | | |
| | ApoB:2100 | 7 | 2.5 |
| | Five 2,6-diaminopurines | | |
| 5-methyluridine:2,6- | ApoB:2100 | 6.9 | 2.7 |
| diaminopurine | One 5-methyluridine:2,6- | | |
| base pair | diaminopurine base pair | | |
| | ApoB:2100 | 4.7 | 1.4 |
| | Three 5- | | |
| | methyluridine:2,6- | | |
| | diaminopurine base pairs | | |
| | ApoB:2100 | 5.2 | 1.5 |
| | Five 5-methyluridine:2,6- | | |
| | diaminopurine base pairs | | |

In general, the results in Table 7 showed that the presence of one or more 5-methyluridines or 2,6-diaminopurines improved the knockdown activity of the dsRNA. Further, the results showed that the one or more 5-methyluridine:2,6-diaminopurine base pairs improved the knockdown activity of the dsRNA. Thus, the presence of one or more 5-methyluridine:2,6-diaminopurine base pairs in the duplex region of a dsRNA improves the potency of the dsRNA.

### Example 4

### drtRNA Duplexes and Cytokine Induction

The effect of drtRNA on cytokine induction is examined. Female BALB/c mice (age 7-9 weeks) are dosed intranasally with about 50 μM drtRNA (formulated in C12-norArg(NH₃+C1-)-C12/DSPE-PEG2000/DSPC/cholesterol at a ratio of 30:1:20:49) or with 605 nmol/kg/day naked dsRNA for three consecutive days. About four hours after the final dose is administered, the mice are sacrificed to collect bronchoalveolar fluid (BALF), and collected blood is processed to serum for evaluation of the cytokine response. Bronchial lavage is performed with 0.5 mL ice-cold 0.3% BSA in saline two times for a total of 1 mL. BALF is spun and supernatants are collected and frozen until cytokine analysis. Blood is collected from the vena cava immediately following euthanasia, is placed into serum separator tubes, and is allowed to clot at room temperature for at least 20 minutes. The samples are processed to serum, are aliquoted into Millipore ULTRAFREE 0.22 μM filter tubes, are spun at 12,000 rpm, frozen on dry ice, and then are stored at −70° C. until analysis. Cytokine analysis of BALF and plasma is performed using the Procarta™ mouse 10-Plex Cytokine Assay Kit (Panomics, Fremont, Calif.) on a Bio-Plex™ array reader. Toxicity parameters are also measured, including body weights, prior to the first dose on day 0 and again on day 3 (just prior to euthanasia). Spleens are harvested and weighed (normalized to final body weight).

### Example 5

### drtRNA Duplexes and “Off-Target” Effects

The off-target profile of dsRNA having at least one 5-methyluridine:2,6-diaminopurine base pair (drtRNA) is examined.

Although siRNA is a powerful technique used to disrupt the expression of target genes, an undesired consequence of this method is that it may also effect the expression of non-target genes (off-target effect). An off-target profile is generated for drtRNA and is compared to dsRNA without a 5-methyluridine:2,6-diaminopurine base pair. Agilent microarrays are used and consisted of 60-mer probe oligonucleotides (targets) representing about 18,500 well-characterized, full-length human genes.

The teachings of all of references cited herein including patents, patent applications and journal articles are incorporated herein in their entirety by reference. Although the foregoing disclosure has been described in detail by way of example for purposes of clarity of understanding, it will be apparent to the artisan that certain changes and modifications may be practiced within the scope of the appended claims which are presented by way of illustration not limitation. In this context, various publications and other references have been cited within the foregoing disclosure for economy of description. It is noted, however, that the various publications discussed herein are incorporated solely for their disclosure prior to the filing date of the present application, and the inventors reserve the right to antedate such disclosure by virtue of prior disclosure.

What is claimed is:

1. A duplex containing ribonucleic acid comprising a double-stranded region having from 10 to 40 base pairs, wherein the double-stranded region contains at least one base pair comprising a 5-methyluridine base paired with a 2,6-diaminopurine.

2. The duplex of claim 1, wherein the double-stranded region is from 15 to 29 base pairs or from 29 to 40 base pairs.

3. The duplex of claim 1, wherein the double-stranded region contains at least two base pairs, each base pair comprising a 5-methyluridine base paired with a 2,6-diaminopurine.

4. The duplex of claim 1, wherein the double-stranded region contains at least three base pairs, each base pair comprising a 5-methyluridines base paired with a 2,6-diaminopurine.

5. The duplex of claim 1 wherein the 5-methyluridine hydrogen bonds with the 2,6-diaminopurine under physiologic pH.

6. The duplex of claim 1 further comprising one or more nucleotide having the following formula: 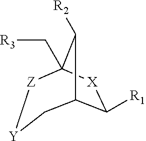
wherein, X is O or CH₂, Y is O, and Z is CH₂;
R₁ is selected from the group consisting of adenine, cytosine, guanine, hypoxanthine, uracil, thymine, 5-methyluridine, 2,6-diaminopurine, C-phenyl, C-naphthyl, inosine, azole carboxamide, 1-β-D-ribofuranosyl-4-nitroindole, 1-β-D-ribofuranosyl-5-nitroindole, 1-β-D-ribofuranosyl-6-nitroindole, or 1-β-D-ribofuranosyl-3-nitropyrrole, and a heterocycle wherein the heterocycle is selected from the group consisting of a substituted 1,3-diazine, unsubstituted 1,3-diazine, and an unsubstituted 7H imidazo[4,5]1,3 diazine; and
R₂, R₃ are independently selected from a group consisting of H, OH, DMTO, TBDMSO, BnO, THPO, AcO, BzO, OP(NiPr₂)O(CH₂)₂CN, OPO₃ H, diphosphate, and triphosphate, wherein R₂ and R₃ together may be PhCHO₂, TIPDSO₂ or DTBSO₂

7. The duplex of claim 1 further comprising one or more locked nucleic acid (LNA) molecules.

8. The duplex of claim 1 further comprising an acyclic nucleotide monomer selected from the group consisting of monomer E, F, G, H, I or J:

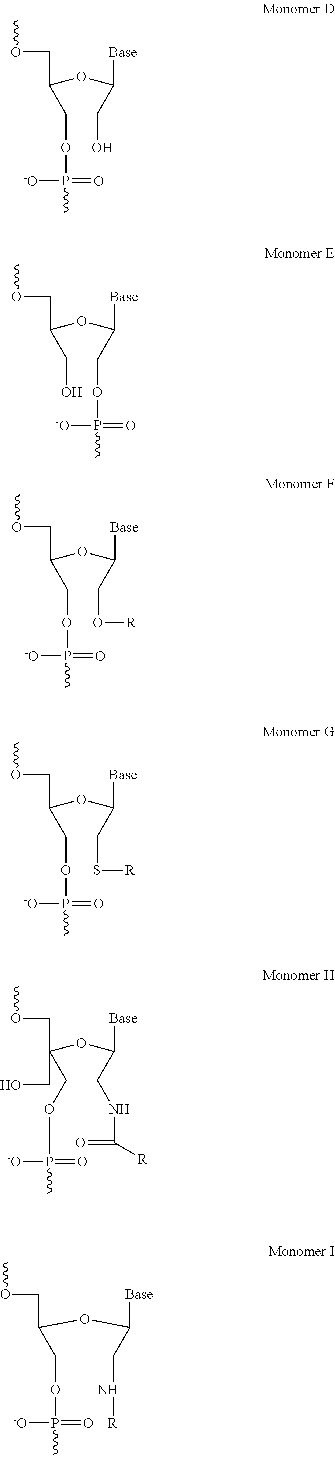

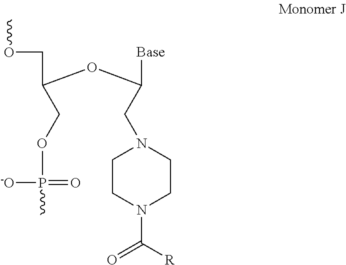

9. The duplex of claim 1 further comprising a universal-binding nucleotide.

10. The duplex of claim 9, wherein the universal-binding nucleotide is a C-phenyl, C-naphthyl, inosine, azole carboxamide, 1-β-D-ribofuranosyl-4-nitroindole, 1-β-D-ribofuranosyl-5-nitroindole, 1-β-D-ribofuranosyl-6-nitroindole, or 1-β-D-ribofuranosyl-3 -nitropyrrole.

11. The duplex of claim 1 further comprising a 2′-sugar substitution.

12. The duplex of claim 11, wherein the 2′-sugar substitution is a 2′-O-methyl, 2′-O-methoxyethyl, or 2′-O-2-methoxyethyl.

13. The duplex of claim 11, wherein the 2′-sugar substitution is a halogen.

14. The duplex of claim 11, wherein the 2′-sugar substitution is a 2′-fluoro.

15. The duplex of claim 11, wherein the 2′-sugar substitution is a 2′-O-allyl.

16. The duplex of claim 1 further comprising at least one modified internucleoside linkage.

17. The duplex of claim 16, wherein the at least one modified internucleoside linkage is independently selected from the group consisting of a phosphorothioate, chiral phosphorothioate, phosphorodithioate, phosphotriester, aminoalkylphosphotriester, methyl phosphonate, alkyl phosphonate, 3′-alkylene phosphonate, 5′-alkylene phosphonate, chiral phosphonate, phosphonoacetate, thiophosphonoacetate, phosphinate, phosphoramidate, 3′-amino phosphoramidate, aminoalkylphosphoramidate, thionophosphoramidate, thionoalkylphosphonate, thionoalkylphosphotriester, selenophosphate, or boranophosphate linkage.

18. The duplex of claim 1, wherein the duplex containing ribonucleic acid has an overhang of one to five nucleotides on one or both 3′-ends of the duplex.

19. The duplex of claim 18, wherein the overhang on one or both 3′-ends of the duplex containing ribonucleic acid has at least one deoxyribonucleotide.

20. The duplex of claim 19, wherein the at least one deoxyribonucleotide is thymidine.

21. The duplex of claim 1, wherein the duplex containing ribonucleic acid has a blunt end at one or both ends of the duplex.

22. The duplex of claim 1, wherein the duplex containing ribonucleic acid has at least two double-stranded regions spaced apart by up to 10 unpaired nucleotides.

23. The duplex of claim 1, wherein the duplex containing ribonucleic acid has at least two double-stranded regions spaced apart by a nick.

24. A duplex containing ribonucleic acid comprising a double-stranded region from 10 to 40 base pairs and a 5-methyluridine base paired with a 2,6-diaminopurine.

25. A duplex containing ribonucleic acid comprising a double-stranded region from 10 to 40 base pairs and at least one nucleotide comprising the structure shown in Formula I base paired with the nucleotide comprising the structure shown in Formula II:

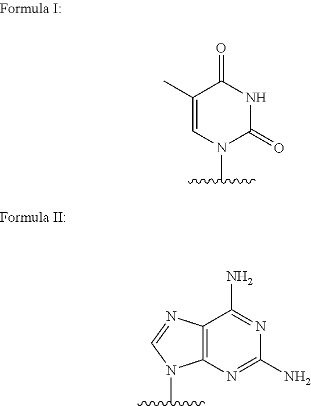

26. A method for activating target gene-specific RNA interference (RNAi), comprising administering a double-stranded ribonucleic acid (dsRNA) that decreases expression of a target gene by RNAi to a cell expressing the target gene, wherein the dsRNA contains a double-stranded region having from 10 to 40 base pairs, and wherein the double-stranded region contains at least one base pair comprising a 5-methyluridine base paired with a 2,6-diaminopurine.

27. A method of preparing a double-stranded ribonucleic acid (dsRNA) that decreases expression of a target gene by RNAi, comprising (a) synthesizing a first strand and a second strand, wherein each strand has a length of from 10 to 60 nucleotides, and wherein the first strand contains at least one 2,6-diaminopurine and the second strand contains at least one 5-methyluridine and (b) combining the first strand and the second strand to form a double-stranded RNA, wherein the double-stranded RNA contains a double-stranded region having from 10 to 40 base pairs, and wherein the double-stranded region contains at least one base pair comprising a 5-methyluridine base paired with a 2,6-diaminopurine.
